(19) United States
(12) Patent Application Publication
(10) Pub. No.: US 2026/0366081 A1
(43) Pub. Date: Nov. 19, 2026

(54) COMPOSITE IMMUNE BIOMARKERS AND USES THEREOF

(71) Applicant: Foundation Medicine, Inc., Boston, MA (US)

(72) Inventors: Khaled A. TOLBA, Portland, OR (US); Meagan Kathleen MONTESION, Somerville, MA (US); David FABRIZIO, Cambridge, MA (US)

(73) Assignee: Foundation Medicine, Inc., Boston, MA (US)

(21) Appl. No.: 19/126,559

(22) PCT Filed: Nov. 3, 2023
(86) PCT No.: PCT/US2023/078695

§ 371 Date: May 1, 2025

Related U.S. Application Data
(60) Provisional application No. 63/422,846, filed on Nov. 4, 2022.

(51) Int. Cl. C12Q 1/6886; G01N 33/575

(52) U.S. Cl. CPC C12Q 1/6886; G01N 33/5758; C12Q 2600/106; C12Q 2600/156; G01N 2333/70532

(57) ABSTRACT

The present disclosure relates to an improved biomarker, termed a composite biomarker score, which integrates several individual biomarkers into a more precise and more accurate predictor of response to immune checkpoint inhibitor therapy. Provided are methods of determining the composite biomarker score and methods relying on the determined composite biomarker score, including diagnostic, prognostic, and therapeutic methods.

### CROSS-REFERENCE TO RELATED APPLICATIONS

This application claims priority benefit to U.S. Provisional Application No. 63/422,846, filed Nov. 4, 2022, the contents of which are hereby incorporated by reference in their entirety for all purposes.

### FIELD OF THE INVENTION

The present disclosure relates generally to methods incorporating a composite biomarker score based on a plurality of individual biomarkers. The composite biomarker score allows for the more precise and more accurate prediction of whether a subject with a cancer is likely to benefit from treatment comprising an immune checkpoint inhibitor.

### BACKGROUND

Rapid advances in tumor immunology and development of immune checkpoint inhibitors (ICI) have transformed the care of solid tumors but yield durable clinical benefit in a fraction of patients with advanced stage disease. Existing biomarkers, such as those identifying PD-L1 expression levels, are not sufficiently predictive of overall survival rates by themselves. Given the level of complexity governing the immune system interaction with the tumor and host micro-environment, a single biomarker does not have prognostic value, and does not inform who will benefit from certain treatments and for how long. There are several checkpoint inhibitor therapies that are either clinically approved or in advanced stage of development for which only a limited set of biomarkers are available to select patients that would be candidates for such therapies. Therefore, new biomarkers of broad prognostic value are useful and urgently needed for identifying those likely to respond to current therapies versus those in need of alternative interventions.

### SUMMARY OF THE INVENTION

Provided herein are methods of identifying a subject with a cancer who will likely benefit from a treatment comprising an immune checkpoint inhibitor, the method comprising determining a composite biomarker score; wherein the composite biomarker score is determined based on at least three of the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Further provided herein are methods of identifying a subject with a cancer who will likely benefit from a treatment comprising an immune checkpoint inhibitor, the method comprising determining a composite biomarker score; wherein the composite biomarker score is determined based on the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e)the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Further provided herein are methods of predicting if a subject with a cancer will likely benefit from a treatment comprising an immune checkpoint inhibitor, the method comprising determining a composite biomarker score; wherein the composite biomarker score is determined based on at least three of the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 pathway gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Further provided herein are methods of predicting if a subject with a cancer will likely benefit from a treatment comprising an immune checkpoint inhibitor, the method comprising determining a composite biomarker score; wherein the composite biomarker score is determined based on the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 pathway gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Further provided herein are methods of stratifying a subject with a cancer for treatment comprising an immune checkpoint inhibitor, the method comprising determining a composite biomarker score; wherein the composite biomarker score is determined based on at least three of the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 pathway gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

In some embodiments of the preceding methods, the composite biomarker score is further based on (h): (h) one or more human leukocyte antigen (HLA) Class I genes of the cancer are determined to exhibit loss of heterozygosity (LOH). In some embodiments, the one or more HLA Class I genes are selected from the group consisting of HLA-A, HLA-B, HLA-C, HLA-DP, HLA-DQ, and HLA-DR. In some embodiments, the one or more HLA Class I genes comprise at least HLA-A, HLA-B, and HLA-C.

In some embodiments of the preceding methods, the method further comprises characterizing the subject as: (i) having a low likelihood of benefitting from the treatment comprising the immune checkpoint inhibitor, (ii) having an indeterminate or medium likelihood of benefitting from the treatment comprising the immune checkpoint inhibitor, or (iii) having a high likelihood of benefitting from the treatment comprising the immune checkpoint inhibitor; wherein the subject is characterized as one of (i)-(iii) based on the determined composite biomarker score.

In some embodiments of the preceding methods, the composite biomarker score is calculated by adding to the score for each of (a)-(g), or (a)-(h), that are met. In some embodiments, each of (a)-(g) or (a)-(h) that are met increments the score by at least 1. In some embodiments, the composite biomarker score starts at 0 and is incremented for each of (a)-(g) or each of (a)-(h) that are met. In some embodiments, each of (a)-(c) and (g) that are met increments the score by at least 1, and wherein each of (d)-(f) that are met decreases the score by 1. In some embodiments, the composite biomarker score starts at 0 and is incremented for each of (a)-(c) and (g) that are met; and is decreased by 1 for each of (d)-(f) that are met. In some embodiments, the determination of whether the subject will likely benefit from the treatment comprising the immune checkpoint inhibitor is based on the composite biomarker score being at least a threshold value.

In some embodiments of the preceding methods, the composite biomarker score is incremented if the TMB score of (a) is at least 10. In some embodiments, the composite biomarker score is incremented by one.

In some embodiments of the preceding methods, the composite biomarker score is incremented if the TMB score of (a) is at least 20. In some embodiments, the composite biomarker score is incremented by two.

In some embodiments of the preceding methods, the composite biomarker score is incremented based on the TMB score of (a) as follows: (i) if the TMB score is less than 10, the composite biomarker score is not incremented, (ii) if the TMB score is at least 10, the composite biomarker score is incremented, and (iii) if the TMB score is at least 20, the composite biomarker score is further incremented over the increment of (ii). In some embodiments, the composite biomarker score is incremented by 1 if (ii) is met and incremented by 2 if both (ii) and (iii) are met.

In some embodiments of the preceding methods, (b) is based on the percent of cells that are positive for PD-L1 being at least 1%. In some embodiments, if (b) is met, the composite biomarker score is incremented by one.

In some embodiments of the preceding methods, (b) is based on the percent of cells that are positive for PD-L1 being at least 50%. In some embodiments, if (b) is met, the composite biomarker score is incremented by two.

In some embodiments of the preceding methods, the composite biomarker score is incremented based on the percent of cells in the sample that are positive for PD-L1 of (b) as follows: (i) if the percent of cells in the sample that are positive for PD-L1 is less than 1%, the composite biomarker score is not incremented, (ii) if the percent of cells in the sample that are positive for PD-L1 is at least 1% and less than 50%, the composite biomarker score is incremented, and (iii) if the percent of cells in the sample that are positive for PD-L1 is at least 50%, the composite biomarker score is further incremented over the increment of (ii). In some embodiments, the composite biomarker score is incremented by 1 if (ii) is met and incremented by 2 if both (ii) and (iii) are met. In some embodiments, the threshold value is 2. In some embodiments, the threshold value is 3. In some embodiments, the threshold value is 4. In some embodiments, the threshold value is 5. In some embodiments, the threshold value is 6.

In some embodiments, the threshold value is 7. In some embodiments, the threshold value is −3. In some embodiments, the threshold value is −2. In some embodiments, the threshold value is −1. In some embodiments, the threshold value is 0. In some embodiments, the threshold value is 1. In some embodiments, if the composite biomarker score is less than the threshold value, the subject is identified as one who will not benefit from treatment with the immune checkpoint inhibitor. In some embodiments, if the composite biomarker score is less than the threshold value, the subject is identified as one who will not benefit from a monotherapy treatment with the immune checkpoint inhibitor.

In some embodiments of the preceding methods, the TMB score is determined based on between about 100 kb to about 10 Mb of genomic sequence.

In some embodiments of the preceding methods, the TMB score is determined based on between about 0.8 Mb to about 1.1 Mb of genomic sequence.

In some embodiments of the preceding methods, the DDR genes comprise one or more of ATM, BRCA2, BRIP1, MRE11, POLE, MSH2, PARP1, MLH1, MSH6, PMS2, BRCA1, PALB2, and BARD1.

In some embodiments of the preceding methods, (g) is based on a cancer-associated mutation reducing gene expression of one or more genes selected from the list consisting of ATM, BRCA2, BRIP1, MRE11, POLE, MSH2, PARP1, MLH1, MSH6, PMS2, BRCA1, PALB2, and BARD1.

In some embodiments of the preceding methods, the DDR genes comprise one or more of ATM, BRCA2, BRIP1, MRE11, POLE, MSH2, and PARP1.

In some embodiments of the preceding methods, the DDR genes comprise one or more of MLH1, MSH2, MSH6, PMS2, ATM, BRCA1, BRCA2, PALB2, BRIP1, and BARD1.

In some embodiments of the preceding methods, (f) is based on a homozygous deletion of CDKN2A.

In some embodiments of the preceding methods, the method further comprises creating a record associated with the subject designating the subject as (i) one who will likely benefit from the treatment comprising an immune checkpoint inhibitor or (ii) one who will likely not benefit from the treatment comprising an immune checkpoint inhibitor, wherein the designation is based on the composite biomarker score.

In some embodiments of the preceding methods, the method further comprises administering the immune checkpoint inhibitor to the identified subject if the subject is determined to be likely to benefit from the treatment comprising the immune checkpoint inhibitor.

In some embodiments of the preceding methods, the cancer is a lung cancer, a melanoma, a bladder cancer, a gastro-esophageal cancer, or a head and neck cancer.

In some embodiments of the preceding methods, the immune checkpoint inhibitor is a PD-1 inhibitor. In some embodiments, the immune checkpoint inhibitor comprises one or more of nivolumab, pembrolizumab, cemiplimab, or dostarlimab.

In some embodiments of the preceding methods, the immune checkpoint inhibitor is a PD-L1 inhibitor. In some embodiments, the immune checkpoint inhibitor comprises one or more of atezolizumab, avelumab, or durvalumab.

In some embodiments of the preceding methods, the immune checkpoint inhibitor is a CTLA-4 inhibitor. In some embodiments, the immune checkpoint inhibitor comprises ipilimumab.

In some embodiments of the preceding methods, the tumor sample is a tissue biopsy sample or a liquid biopsy sample. In some embodiments, the sample is a tissue biopsy and comprises a tumor biopsy or a tumor specimen. In some embodiments, the sample is a liquid biopsy sample and comprises circulating tumor cells, blood, serum, plasma, cerebrospinal fluid, sputum, stool, urine, or saliva. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or both.

In some embodiments of the preceding methods, the sample comprises cells and/or nucleic acids from the cancer. In some embodiments, the sample comprises mRNA, DNA, circulating tumor DNA (ctDNA), cell-free DNA, cell-free RNA from the cancer, or any combination thereof.

In some embodiments of the preceding methods, the LOH status is determined based on sequence read data derived from sequencing nucleic acid molecules extracted from the sample. In some embodiments, the LOH status sequencing comprises use of a massively parallel sequencing (MPS) technique, whole genome sequencing (WGS), whole exome sequencing, targeted sequencing, direct sequencing, next-generation sequencing (NGS), or a Sanger sequencing technique. In some embodiments, the sequencing comprises: (a) providing a plurality of nucleic acid molecules obtained from the sample, wherein the plurality of nucleic acid molecules comprises a mixture of tumor nucleic acid molecules and non-tumor nucleic acid molecules; (b) optionally, ligating one or more adapters onto one or more nucleic acid molecules from the plurality of nucleic acid molecules; (c) amplifying nucleic acid molecules from the plurality of nucleic acid molecules; (d) optionally, capturing nucleic acid molecules from the amplified nucleic acid molecules, wherein the captured nucleic acid molecules are captured from the amplified nucleic acid molecules by hybridization to one or more bait molecules; and (e) sequencing, by a sequencer, the captured nucleic acid molecules to obtain a plurality of sequence reads corresponding to one or more genomic loci within a subgenomic interval in the sample. In some embodiments, the adapters comprise one or more of amplification primer sequences, flow cell adapter hybridization sequences, unique molecular identifier sequences, substrate adapter sequences, or sample index sequences. In some embodiments, amplifying nucleic acid molecules comprises performing a polymerase chain reaction (PCR) technique, a non-PCR amplification technique, or an isothermal amplification technique. In some embodiments, the one or more bait molecules comprise one or more nucleic acid molecules, each comprising a region that is complementary to a region of a captured nucleic acid molecule. In some embodiments, the one or more bait molecules each comprise a capture moiety. In some embodiments, the capture moiety is biotin.

In some embodiments of the preceding methods, the subject is a human.

Further provided herein are methods of treating a subject having a cancer, wherein the subject is identified as one who will benefit from an immune checkpoint inhibitor according to the method of any of the preceding methods, the method comprising administering the immune checkpoint inhibitor to the subject.

In some embodiments of the preceding methods, the composite biomarker score is calculated by adding to the score for each positive biomarker and subtracting from the score for each negative biomarker for each of (a)-(g), or (a)-(h), that are met. In some embodiments of the preceding methods, each of (a)-(g) or (a)-(h) that are met increments or decreases the score by at least 1.

Further provided herein are methods of identifying a subject with a cancer who will likely benefit from a treatment comprising an immune checkpoint inhibitor, the method comprising determining a composite biomarker score; wherein the composite biomarker score is determined based on at least three of the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

In some embodiments, the method comprises determining a composite biomarker score; wherein the composite biomarker score is determined based on the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor. In some embodiments, the composite biomarker score is based on at least three of (a)-(g) and further based on (h): (h) one or more human leukocyte antigen (HLA) Class I genes of the cancer are determined to exhibit loss of heterozygosity (LOH). In some embodiments, the one or more HLA Class I genes are selected from the group consisting of HLA-A, HLA-B, HLA-C, HLA-DP, HLA-DQ, and HLA-DR. In some embodiments, the method further comprises characterizing the subject as: (i) having a low likelihood of benefitting from the treatment comprising the immune checkpoint inhibitor, (ii) having an indeterminate or medium likelihood of benefitting from the treatment comprising the immune checkpoint inhibitor, or (iii) having a high likelihood of benefitting from the treatment comprising the immune checkpoint inhibitor; wherein the subject is characterized as one of (i)-(iii) based on the determined composite biomarker score. In some embodiments, the composite biomarker score is calculated by adding to the score for each positive biomarker and subtracting from the score for each negative biomarker for each of (a)-(g), or (a)-(h), that are met. In some embodiments, each of (a)-(g) or (a)-(h) that are met increments or decreases the score by at least 1. In some embodiments, the composite biomarker score starts at 0 and is incremented or decreased for each of (a)-(g) or each of (a)-(h) that are met. In some embodiments, the determination of whether the subject will likely benefit from the treatment comprising the immune checkpoint inhibitor is based on the composite biomarker score being at least a threshold value. In some embodiments, the composite biomarker score is incremented based on the TMB score of (a) as follows: (i) if the TMB score is less than 10, the composite biomarker score is not incremented or decreased, (ii) if the TMB score is at least 10, the composite biomarker score is incremented, and (iii) if the TMB score is at least 20, the composite biomarker score is further incremented over the increment of (ii). In some embodiments, the composite biomarker score is incremented by 1 if (ii) is met and incremented by a total of 2 if both (ii) and (iii) are met. In some embodiments, the composite biomarker score is incremented based on the percent of cells in the sample that are positive for PD-L1 of (b) as follows: (i) if the percent of cells in the sample that are positive for PD-L1 is less than 1%, the composite biomarker score is not incremented or decreased, (ii) if the percent of cells in the sample that are positive for PD-L1 is at least 1% and less than 50%, the composite biomarker score is incremented, and (iii) if the percent of cells in the sample that are positive for PD-L1 is at least 50%, the composite biomarker score is further incremented over the increment of (ii). In some embodiments, the composite biomarker score is incremented by 1 if (ii) is met and incremented by a total of 2 if both (ii) and (iii) are met. In some embodiments, if the composite biomarker score is less than the threshold value, the subject is identified as one who will not benefit from treatment with the immune checkpoint inhibitor. In some embodiments, the DDR genes comprise one or more of ATM, BRCA2, BRIP1, MRE11, POLE, MSH2, PARP1, MLH1, MSH6, PMS2, BRCA1, PALB2, and BARD1; and wherein (g) is based on a cancer-associated mutation reducing gene expression of one or more genes selected from the list consisting of ATM, BRCA2, BRIP1, MRE11, POLE, MSH2, PARP1, MLH1, MSH6, PMS2, BRCA1, PALB2, and BARD1. In some embodiments, (f) is based on a homozygous deletion of CDKN2A. In some embodiments, the method further comprises administering the immune checkpoint inhibitor to the identified subject if the subject is determined to be likely to benefit from the treatment comprising the immune checkpoint inhibitor. In some embodiments, the cancer is a lung cancer, a melanoma, a bladder cancer, a gastro-esophageal cancer, or a head and neck cancer. In some embodiments, the immune checkpoint inhibitor is a PD-1 inhibitor, a PD-L1 inhibitor, or a CTLA-4 inhibitor. In some embodiments, the tumor sample is a tissue biopsy sample or a liquid biopsy sample. In some embodiments, the sample is a tissue biopsy and comprises a tumor biopsy or a tumor specimen. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or both. In some embodiments, the subject is a human. Further provided herein are methods of treating a subject having a cancer, wherein the subject is identified as one who will benefit from an immune checkpoint inhibitor according to the preceding methods, the method comprising administering the immune checkpoint inhibitor to the subject.

### BRIEF DESCRIPTION OF THE DRAWINGS

FIG. 1A shows a clinical trial flow chart for advanced non-small-cell lung carcinoma (NSCLC) patients who received atezolizumab. Patients initially received platinum therapy, and were further divided into groups based on squamous or non-squamous NSCLC and determined to be responders or non-responders to the atezolizumab based on disease progression. Patients were wild-type for EGFR and ALK, and also did not possess known or likely pathogenic driver mutations in BRAF, MET, ROS1, and RET.

FIG. 1B shows a clinical trial flow chart for advanced non-small-cell lung carcinoma (NSCLC) patients who received docetaxel. Patients initially received platinum therapy and were further divided into groups based on squamous or non-squamous NSCLC, and determined to be responders or non-responders based on disease progression. Patients were wild-type for EGFR and ALK, and also did not possess known or likely pathogenic driver mutations in BRAF, MET, ROS1, and RET.

FIG. 2A-B shows Kaplan-Meier curves demonstrating overall survival (OS) of patients who received atezolizumab based on the indicated individual biomarkers (positive or negative biomarkers). ARID1A (top left), CDKN2A (top right), NFE2L2/KEAP1 (bottom left), and STK11 (bottom right) biomarkers are shown in FIG. 2A. TMB score (top left and top middle), percent of cells positive for PD-L1 (bottom left and bottom right), and DNA damage response (top right) biomarkers are shown in FIG. 2B. “PD-L1 High Positive” is determined based on at least 50% of cells in a tumor sample being positive for PD-L1 as assessed by IHC. “PD-L1 positive” is based on at least 1% and less than 50% of cells in a tumor sample being positive for PD-L1 as determined by IHC. Hazard ratio (HR) and p values are reported. Time of median survival for each curve is indicated by dotted lines.

FIG. 3 shows Kaplan-Meier curves demonstrating the overall survival (OS) of patients who received atezolizumab stratified by composite biomarker scores. Logrank P represents the p-value for comparing the survival distributions of the samples. Time of median survival for each curve is indicated by dotted lines.

FIG. 4 shows the hazard ratio of patients who received atezolizumab based on composite biomarker scores compared to a reference biomarker score of 0. Values for hazard ratios (HR), Akaike Information Criterion (AIC), and 95% concordance index are reported.

FIG. 5 shows a Kaplan-Meier curve demonstrating the overall survival (OS) of patients who received atezolizumab based on binned composite biomarker score. Logrank P represents the p-value for comparing the survival distributions of the samples. Time of median survival for each curve is indicated by dotted lines.

FIG. 6 shows hazard ratio of patients who received atezolizumab based on binned composite biomarker scores. Values for hazard ratios (HR), Akaike Information Criterion (AIC), and 95% concordance index are reported.

FIG. 7 shows Kaplan-Meier curves demonstrating the overall survival (OS) of patients who received docetaxel based on binned composite biomarker score. Logrank P represents the p-value for comparing the survival distributions of the samples. Time of median survival for each curve is indicated by dotted lines.

FIG. 8 shows hazard ratio of patients who received docetaxel based on binned composite biomarker score. Values for hazard ratios (HR), Akaike Information Criterion (AIC), and 95% concordance index are reported.

FIG. 9 shows Kaplan-Meier curves demonstrating the overall survival (OS) of NSCLC patients who received docetaxel or atezolizumab over time based on binned composite biomarker score. Hazard ratio values (HR) and p values are reported. Time of median survival for each curve is indicated by dotted lines.

FIG. 10 shows a flow chart for selecting samples from patients with advanced non-small-cell lung carcinoma (NSCLC). Specimens were profiled using one of two different tissue-based comprehensive genomic profiling tests. Patients received second line immunotherapy (IO) monotherapy. Patients were wild-type for EGFR and ALK, and also did not possess known or likely pathogenic driver mutations in BRAF, MET, ROS1, and RET. Samples were then further sub-divided into squamous versus non-squamous cancer.

FIG. 11 shows Kaplan-Meier curves demonstrating the overall survival (OS) of NSCLC patients who received second line immunotherapy (IO) monotherapy stratified by single biomarker scores. Logrank P represents the p-value for comparing the survival distributions of the samples. Time of median survival for each curve is indicated by dotted lines.

FIG. 12 shows the hazard ratio of NSCLC patients who received second line immunotherapy (IO) (monotherapy) based on single biomarker scores compared to a reference biomarker score of +4. Values for hazard ratios (HR), Akaike Information Criterion (AIC), and 95% concordance index are reported.

FIG. 13 shows Kaplan-Meier curves demonstrating the overall survival (OS) of NSCLC patients who received second line immunotherapy (IO) (monotherapy) stratified by composite biomarker scores. Logrank P represents the p-value for comparing the survival distributions of the samples. Time of median survival for each curve is indicated by dotted lines.

FIG. 14 shows the hazard ratio of NSCLC patients who received second line immunotherapy (IO) (monotherapy) based on composite biomarker scores compared to a reference biomarker score of medium (0-2). Values for hazard ratios (HR), Akaike Information Criterion (AIC), and 95% concordance index are reported.

FIG. 15 shows Kaplan-Meier curves demonstrating the overall survival (OS) of NSCLC (non-squamous) patients who received second line immunotherapy (IO) monotherapy stratified by composite biomarker scores. Logrank P represents the p-value for comparing the survival distributions of the samples. Time of median survival for each curve is indicated by dotted lines.

FIG. 16 shows the hazard ratio of NSCLC (non-squamous) patients who received second line IO monotherapy based on composite biomarker scores compared to a reference biomarker score of medium (0-2). Values for hazard ratios (HR), Akaike Information Criterion (AIC), and 95% concordance index are reported.

FIG. 17 shows Kaplan-Meier curves demonstrating the overall survival (OS) of NSCLC (squamous) patients who received second line immunotherapy (IO) monotherapy stratified by composite biomarker scores. Logrank P represents the p-value for comparing the survival distributions of the samples. Time of median survival for each curve is indicated by dotted lines.

FIG. 18 shows the hazard ratio of NSCLC (squamous) patients who received second line IO monotherapy based on composite biomarker scores compared to a reference biomarker score of medium (0-2). Values for hazard ratios (HR), Akaike Information Criterion (AIC), and 95% concordance index are reported.

FIG. 19 shows a flow chart for selecting samples from patients with advanced non-small-cell lung carcinoma (NSCLC). Specimens were profiled using one of two different tissue-based comprehensive genomic profiling tests. Patients received second line immunotherapy (IO) (monotherapy). Patients were wild-type for EGFR and ALK, and also did not possess known or likely pathogenic driver mutations in BRAF, MET, ROS1, and RET. Samples were further filtered by whether the patient had PD-L1 TPS data available, then further sub-divided into squamous versus non-squamous NSCLC.

FIG. 20 shows Kaplan-Meier curves demonstrating the overall survival (OS) of NSCLC patients with PD-L1 TPS data available, and received second line immunotherapy (IO) (monotherapy) stratified by composite biomarker scores. Logrank P represents the p-value for comparing the survival distributions of the samples. Time of median survival for each curve is indicated by dotted lines.

FIG. 21 shows the hazard ratio of NSCLC patients with PD-L1 TPS data available, and received second line immunotherapy (IO) (monotherapy) based on composite biomarker scores compared to a reference biomarker score of medium (0-2). Values for hazard ratios (HR), Akaike Information Criterion (AIC), and 95% concordance index are reported.

FIG. 22 shows Kaplan-Meier curves demonstrating the overall survival (OS) of NSCLC (non-squamous) patients who had PD-L1 TPS data available and received second line immunotherapy (IO) monotherapy stratified by composite biomarker scores. Logrank P represents the p-value for comparing the survival distributions of the samples. Time of median survival for each curve is indicated by dotted lines.

FIG. 23 shows the hazard ratio of NSCLC (non-squamous) patients who had PD-L1 TPS data available, and received second line immunotherapy (IO) (monotherapy) based on composite biomarker scores compared to a reference biomarker score of medium (0-2). Values for hazard ratios (HR), Akaike Information Criterion (AIC), and 95% concordance index are reported.

FIG. 24 shows Kaplan-Meier curves demonstrating the overall survival (OS) of NSCLC (squamous) patients who had PD-L1 TPS data available, and received second line immunotherapy (IO) (monotherapy) stratified by composite biomarker scores. Logrank P represents the p-value for comparing the survival distributions of the samples. Time of median survival for each curve is indicated by dotted lines.

FIG. 25 shows the hazard ratio of NSCLC (squamous) patients who had PD-L1 TPS data available, and received second line immunotherapy (IO) (monotherapy) based on composite biomarker scores compared to a reference biomarker score of medium (0-2). Values for hazard ratios (HR), Akaike Information Criterion (AIC), and 95% concordance index are reported.

FIG. 26 shows the hazard ratios of NSCLC (squamous) patients who received second line immunotherapy (IO) (monotherapy) based on composite biomarker scores (compared to a reference biomarker score of medium (0-2)) and whether the cancer was squamous cell carcinoma (SCC; e.g., SCC or non-SCC). Values for hazard ratios (HR), Akaike Information Criterion (AIC), and 95% concordance index are reported.

FIG. 27 shows the hazard ratios of NSCLC (squamous) patients who had PD-L1 TPS data available, and received second line immunotherapy (IO) (monotherapy) based on composite biomarker scores (compared to a reference biomarker score of medium (0-2)) and whether the cancer was squamous cell carcinoma (SCC; e.g., SCC or non-SCC). Values for hazard ratios (HR), Akaike Information Criterion (AIC), and 95% concordance index are reported.

### DETAILED DESCRIPTION

The present disclosure provides a composite biomarker score which integrates certain individual biomarkers and allows for the more accurate and precise prediction of a subject's response to immune checkpoint inhibitor therapy. Many biomarkers by themselves do not identify patients that will benefit from a treatment comprising an immune checkpoint inhibitor with sufficient accuracy and precision. The individual biomarkers described herein were selected for both their predictive power, when integrated to a composite biomarker score, and their suitability to clinical practice. The composite biomarker score is generated using the individual biomarkers, which increment the composite biomarker score. Increments can be either positive or negative. For example, in an exemplary embodiment, a positive biomarker score above a threshold value indicates the subject is a candidate for a particular therapy (such as an immune checkpoint inhibitor therapy). However, if one of the individual biomarkers increments the composite biomarker score below the threshold value, then the subject is not a candidate for the therapy. By generating the composite biomarker score, an appropriate therapy may be selected. Once selected, the appropriate therapy may be administered to the subject.

To illustrate a particular use case for the composite biomarker score, the present disclosure provides methods for identifying a subject with a cancer who will likely benefit from a treatment comprising an immune checkpoint inhibitor, the method comprising determining a composite biomarker score. In an exemplary embodiment, the composite biomarker score is determined based on a series of individual biomarker conditions, wherein each condition that is met increments the composite biomarker score. For example, in this exemplary embodiment, one biomarker is the tumor mutational burden (TMB) score of a tumor sample from the subject having the cancer. The condition is whether the TMB score is at least 10 mutations/MB and less than 20 mutations/MB. A further condition is whether the TMB score is at least 20 mutations/MB. In this exemplary embodiment, if the TMB score is less than 10 mutations/MB, the composite biomarker score is not changed; if the TMB score is at least 10 mutations/MB and less than 20 mutations/MB, the composite biomarker score is incremented by 1; and if the TMB score is at least 20 mutations/MB, the composite biomarker score is incremented by 2. Each biomarker and associated condition is assessed and used to increment (or not increment) the composite biomarker score. Once each individual biomarker is assessed and integrated to the composite biomarker score, if the composite biomarker score is at least a threshold value, then the subject being assessed may be deemed likely to benefit from treatment comprising an immune checkpoint inhibitor (ICI). For example, in this exemplary embodiment, if the composite biomarker score is between 0 and 2, the subject would be deemed unlikely to benefit from the ICI therapy; if the composite biomarker score is between 3 and 4, the subject would be deemed as having an intermediate (or indeterminate, or medium) likelihood to respond to the ICI therapy; and if the composite biomarker score was at least 5, the subject would be deemed as likely (e.g., having a high likelihood) to benefit from the ICI therapy. The composite biomarker score represents a likelihood that the subject having a cancer will respond to ICI therapy (such as, for example, ICI monotherapy, as opposed to ICI therapy in combination with an additional anti-cancer agent or by a non-ICI therapy). In this example, subjects may be divided into three “bins” of 0-2, 3-4, and 5+, wherein each bin represents an increasing likelihood (for example, from low likelihood, to intermediate (or indeterminate, or medium) likelihood, to high likelihood) that the subject will respond to an immune checkpoint inhibitor therapy for their cancer. In another exemplary embodiment, if the composite biomarker score is between 0 and 2, the subject would be deemed to have a defined overall survival probability post ICI therapy; if the composite biomarker score is 3 or higher, the subject would be deemed as having an increased overall survival post ICI therapy compared to individuals who had a composite biomarker score between 0 and 2; and if the composite biomarker score was −1 or lower, the subject would be deemed likely to have an overall survival after ICI therapy compared to an individual with a biomarker score between 0-2. The composite biomarker score represents an overall survival benefit that the subject having a cancer will have after administration of an ICI therapy (such as, for example, second line ICI monotherapy. In another example, subjects may be divided into three “bins” of −1 or less, 0-2, and 3+, wherein each bin represents an increasing likelihood (for example, from low likelihood, to intermediate (or indeterminate, or medium) likelihood, to high likelihood) that the subject will respond to an immune checkpoint inhibitor therapy for their cancer. Accordingly, provided herein are both methods for determining the composite biomarker score and methods applying the determined composite biomarker score (such as methods of treatment where the treatment is based on the determined composite biomarker score, for example).

Disclosed herein are methods of identifying a subject with a cancer who will likely benefit from a treatment comprising an immune checkpoint inhibitor, the method comprising determining a composite biomarker score and, based on the determined composite biomarker score, determining whether the subject will likely benefit from the treatment comprising the immune checkpoint inhibitor. In some embodiments, the composite biomarker score is determined based on, at least in part, a tumor mutational burden (TMB) score. In some embodiments, the composite biomarker score is determined based on, at least in part, the percent of cells in a tumor sample that are positive for PD-L1. In some embodiments, the composite biomarker score is determined based on, at least in part, the presence of a cancer-associated mutation in a ARID1A gene in a tumor sample. In some embodiments, the composite biomarker score is determined based on, at least in part, the presence of a cancer-associated mutation in a KEAP1/NFE2L2 pathway gene in the sample. In some embodiments, the composite biomarker score is determined based on, at least in part, the presence of a cancer-associated mutation in a STK11/LKB1 gene in a tumor sample. In some embodiments, the composite biomarker score is determined based on, at least in part, the presence of a cancer-associated mutation in a CDKN2A gene in a tumor sample. In some embodiments, the composite biomarker score is determined based on, at least in part, the presence of a cancer-associated mutation in a DNA damage response (DDR) gene in a tumor sample (such as the DDR genes ATM, BRCA2, BRIP1, MRE11, POLE, MSH2, PARP1, MLH1, MSH6, PMS2, BRCA1, PALB2, and/or BARD1). In some embodiments, the composite biomarker score is determined based on, at least in part, the status of one or more human leukocyte antigen (HLA) genes (such as any or all of HLA-A, HLA-B, HLA-C, HLA-DP, HLA-DQ, and/or HLA-DR) of the cancer being determined to exhibit loss of heterozygosity.

Also disclosed herein are methods of identifying a subject with a cancer who will likely benefit from a treatment comprising an immune checkpoint inhibitor, the method comprising determining a composite biomarker score; wherein the composite biomarker score is determined based on at least tumor mutational burden (TMB) and the percent of cells in a tumor sample that are positive for PD-L1. In some embodiments, the composite biomarker score is also determined based on at least one of the following biomarkers: the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene in the sample (such as the DDR genes BRCA2, BRIP1, MRE11, POLE, MSH2, PARP1, MLH1, MSH6, PMS2, ATM, BRCA1, PALB2, and/or BARD1); and determining, based on the determined composite biomarker score, whether the subject will likely benefit from the treatment comprising the immune checkpoint inhibitor. In some embodiments, the composite biomarker score is determined based on, at least in part, the status of one or more human leukocyte antigen (HLA) genes (such as any or all of HLA-A, HLA-B, HLA-C, HLA-DP, HLA-DQ, and/or HLA-DR) of the cancer being determined to exhibit loss of heterozygosity.

Disclosed herein are methods of identifying a subject with a cancer who will likely benefit from a treatment comprising an immune checkpoint inhibitor, the method comprising determining a composite biomarker score; wherein the composite biomarker score is determined based on at least three of the following individual biomarkers: a tumor mutational burden (TMB) score of a tumor sample associated with the cancer, percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, and/or PARP1) in the sample; and determining, based on the determined composite biomarker score, whether the subject will likely benefit from the treatment comprising the immune checkpoint inhibitor. In some embodiments, the composite biomarker score is determined based on, at least in part, the status of one or more human leukocyte antigen (HLA) genes (such as any or all of HLA-A, HLA-B, HLA-C, HLA-DP, HLA-DQ, and/or HLA-DR) of the cancer being determined to exhibit loss of heterozygosity.

Disclosed herein are methods of identifying a subject with a cancer who will likely benefit from a treatment comprising an immune checkpoint inhibitor, the method comprising determining a composite biomarker score; wherein the composite biomarker score is determined based on at least four of the following individual biomarkers: a tumor mutational burden (TMB) score of a tumor sample associated with the cancer, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor. In some embodiments, the composite biomarker score is determined based on, at least in part, the status of one or more human leukocyte antigen (HLA) genes (such as any or all of HLA-A, HLA-B, HLA-C, HLA-DP, HLA-DQ, and/or HLA-DR) of the cancer being determined to exhibit loss of heterozygosity.

Disclosed herein are methods of identifying a subject with a cancer who will likely benefit from a treatment comprising an immune checkpoint inhibitor, the method comprising determining a composite biomarker score; wherein the composite biomarker score is determined based on at least five of the following individual biomarkers: a tumor mutational burden (TMB) score of a tumor sample associated with the cancer, percent of cells in a tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor. In some embodiments, the composite biomarker score is further determined based on the status of one or more human leukocyte antigen (HLA) genes (such as any or all of HLA-A, HLA-B, HLA-C, HLA-DP, HLA-DQ, and/or HLA-DR) of the cancer being determined to exhibit loss of heterozygosity.

Disclosed herein are methods of identifying a subject with a cancer who will likely benefit from a treatment comprising an immune checkpoint inhibitor, the method comprising determining a composite biomarker score; wherein the composite biomarker score is determined based on at least six of the following individual biomarkers: a tumor mutational burden (TMB) score of a tumor sample associated with the cancer, the percent of cells in a tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor. In some embodiments, the composite biomarker score is further determined based on the status of one or more human leukocyte antigen (HLA) genes (such as any or all of HLA-A, HLA-B, HLA-C, HLA-DP, HLA-DQ, and/or HLA-DR) of the cancer being determined to exhibit loss of heterozygosity.

Disclosed herein are methods of identifying a subject with a cancer who will likely benefit from a treatment comprising an immune checkpoint inhibitor, the method comprising determining a composite biomarker score; wherein the composite biomarker score is determined based on at least seven of the following individual biomarkers: a tumor mutational burden (TMB) score of a tumor sample associated with the cancer, percent of cells in a tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor. In some embodiments, the composite biomarker score is further determined based on the status of one or more human leukocyte antigen (HLA) genes (such as any or all of HLA-A, HLA-B, HLA-C, HLA-DP, HLA-DQ, and/or HLA-DR) of the cancer being determined to exhibit loss of heterozygosity.

In some embodiments, the composite biomarker score is further determined based on the status of one or more human leukocyte antigen (HLA) genes of the cancer exhibiting loss of heterozygosity (LOH). In some embodiments, one or more of the HLA genes comprise at least HLA-A, HLA-B, and HLA-C. In some embodiments, one or more of the HLA genes comprise one or more of HLA-A, HLA-B, HLA-C, HLA-DP, HLA-DQ, and/or HLA-DR. In some embodiments, one or more of the HLA genes comprise each of HLA-A, HLA-B, HLA-C, HLA-DP, HLA-DQ, and HLA-DR.

Disclosed herein are methods of predicting if a subject with a cancer will likely benefit from a treatment comprising an immune checkpoint inhibitor, the method comprising a composite biomarker score and, based on the determined composite biomarker score, determining whether the subject will likely benefit from the treatment comprising the immune checkpoint inhibitor. In some embodiments, the composite biomarker score is determined based on, at least in part, a tumor mutational burden (TMB) score of a tumor sample associated with the cancer. In some embodiments, the composite biomarker score is determined based on, at least in part, the percent of cells in a tumor sample that are positive for PD-L1. In some embodiments, the composite biomarker score is determined based on, at least in part, the presence of a cancer-associated mutation in a ARID1A gene in a tumor sample. In some embodiments, the composite biomarker score is determined based on, at least in part, the presence of a cancer-associated mutation in a KEAP1/NFE2L2 pathway gene in the sample. In some embodiments, the composite biomarker score is determined based on, at least in part, the presence of a cancer-associated mutation in a STK11/LKB1 gene in a tumor sample. In some embodiments, the composite biomarker score is determined based on, at least in part, the presence of a cancer-associated mutation in a CDKN2A gene in a tumor sample. In some embodiments, the composite biomarker score is determined based on, at least in part, the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in a tumor sample.

Disclosed herein are methods of predicting if a subject with a cancer will likely benefit from a treatment comprising an immune checkpoint inhibitor, the method comprising determining a composite biomarker score; wherein the composite biomarker score is determined based on at least a tumor mutational burden (TMB) score of a tumor sample associated with the cancer and the percent of cells in a tumor sample that are positive for PD-L1. In some embodiments, the composite biomarker score is also determined based on at least one of the following biomarkers: the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of predicting if a subject with a cancer will likely benefit from a treatment comprising an immune checkpoint inhibitor, the method comprising determining a composite biomarker score; wherein the composite biomarker score is determined based on at least three of the following individual biomarkers: a tumor mutational burden (TMB) score of a tumor sample associated with the cancer, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of predicting if a subject with a cancer will likely benefit from a treatment comprising an immune checkpoint inhibitor, the method comprising determining a composite biomarker score; wherein the composite biomarker score is determined based on at least four of the following individual biomarkers: a tumor mutational burden (TMB) score of a tumor sample associated with the cancer, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of predicting if a subject with a cancer will likely benefit from a treatment comprising an immune checkpoint inhibitor, the method comprising determining a composite biomarker score; wherein the composite biomarker score is determined based on at least five of the following individual biomarkers: tumor mutational burden (TMB), percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of predicting if a subject with a cancer will likely benefit from a treatment comprising an immune checkpoint inhibitor, the method comprising determining a composite biomarker score; wherein the composite biomarker score is determined based on at least six of the following individual biomarkers: a tumor mutational burden (TMB) score of a tumor sample associated with the cancer, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of predicting if a subject with a cancer will likely benefit from a treatment comprising an immune checkpoint inhibitor, the method comprising determining a composite biomarker score; wherein the composite biomarker score is determined based on at least seven of the following individual biomarkers: a tumor mutational burden (TMB) score of a tumor sample associated with the cancer, percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

In some embodiments, the composite biomarker score is further determined based on the status of one or more human leukocyte antigen (HLA) genes of the cancer exhibiting loss of heterozygosity (LOH). In some embodiments, one or more of the HLA genes comprise at least HLA-A, HLA-B, and HLA-C. In some embodiments, one or more of the HLA genes comprise one or more of HLA-A, HLA-B, HLA-C, HLA-DP, HLA-DQ, and/or HLA-DR. In some embodiments, one or more of the HLA genes comprise each of HLA-A, HLA-B, HLA-C, HLA-DP, HLA-DQ, and HLA-DR.

Disclosed herein are methods of stratifying a subject with a cancer for treatment comprising an immune checkpoint inhibitor, the method comprising a composite biomarker score and, based on the determined composite biomarker score, determining whether the subject will likely benefit from the treatment comprising the immune checkpoint inhibitor. In some embodiments, the composite biomarker score is determined based on, at least in part, a tumor mutational burden (TMB) score. In some embodiments, the composite biomarker score is determined based on, at least in part, the percent of cells in a tumor sample that are positive for PD-L1. In some embodiments, the composite biomarker score is determined based on, at least in part, the presence of a cancer-associated mutation in a ARID1A gene in a tumor sample. In some embodiments, the composite biomarker score is determined based on, at least in part, the presence of a cancer-associated mutation in a KEAP1/NFE2L2 pathway gene in the sample. In some embodiments, the composite biomarker score is determined based on, at least in part, the presence of a cancer-associated mutation in a STK11/LKB1 gene in a tumor sample. In some embodiments, the composite biomarker score is determined based on, at least in part, the presence of a cancer-associated mutation in a CDKN2A gene in a tumor sample. In some embodiments, the composite biomarker score is determined based on, at least in part, the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in a tumor sample.

Disclosed herein are methods of stratifying a subject with a cancer for treatment comprising an immune checkpoint inhibitor, the method comprising determining a composite biomarker score; wherein the composite biomarker score is determined based on at least tumor mutational burden (TMB) score and the percent of cells in a tumor sample that are positive for PD-L1. In some embodiments, the composite biomarker score is also determined based on at least one of the following biomarkers: the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of stratifying a subject with a cancer for treatment comprising an immune checkpoint inhibitor, the method comprising determining a composite biomarker score; wherein the composite biomarker score is determined based on at least three of the following individual biomarkers: a tumor mutational burden (TMB) score, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor. In some embodiments, the methods further comprise determining the composite biomarker score based on the status of one or more human leukocyte antigen (HLA) genes of the cancer are determined to exhibit loss of heterozygosity (LOH).

Disclosed herein are methods of stratifying a subject with a cancer for treatment comprising an immune checkpoint inhibitor, the method comprising determining a composite biomarker score; wherein the composite biomarker score is determined based on at least four of the following individual biomarkers: a tumor mutational burden (TMB) score, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of stratifying a subject with a cancer for treatment comprising an immune checkpoint inhibitor, the method comprising determining a composite biomarker score; wherein the composite biomarker score is determined based on at least five of the following individual biomarkers: a tumor mutational burden (TMB) score, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of stratifying a subject with a cancer for treatment comprising an immune checkpoint inhibitor, the method comprising determining a composite biomarker score; wherein the composite biomarker score is determined based on at least six of the following individual biomarkers: a tumor mutational burden (TMB) score, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of stratifying a subject with a cancer for treatment comprising an immune checkpoint inhibitor, the method comprising determining a composite biomarker score; wherein the composite biomarker score is determined based on at least seven of the following individual biomarkers: a tumor mutational burden (TMB) score, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

Disclosed herein are methods of predicting if a subject with a cancer will likely benefit from a treatment comprising an immune checkpoint inhibitor, the method comprising determining a composite biomarker score; wherein the composite biomarker score is determined based on at least seven of the following individual biomarkers: a tumor mutational burden (TMB) score, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

In some of the disclosed embodiments, the composite biomarker score is further determined based on the status of one or more human leukocyte antigen (HLA) genes of the cancer exhibiting loss of heterozygosity (LOH). In some embodiments, one or more of the HLA genes comprise at least HLA-A, HLA-B, and HLA-C. In some embodiments, one or more of the HLA genes comprise one or more of HLA-A, HLA-B, HLA-C, HLA-DP, HLA-DQ, and/or HLA-DR. In some embodiments, one or more of the HLA genes comprise each of HLA-A, HLA-B, HLA-C, HLA-DP, HLA-DQ, and HLA-DR.

In some embodiments, the methods further comprise characterizing the subject as having (i) a low likelihood of benefitting from the treatment comprising the immune checkpoint inhibitor, (ii) having an indeterminate likelihood (or an intermediate likelihood, such as a medium likelihood) of benefitting from the treatment comprising the immune checkpoint inhibitor, or (iii) having a high likelihood of benefitting from the treatment comprising the immune checkpoint inhibitor; wherein the subject is characterized as one of (i)-(iii) based on the determined composite biomarker score. In some embodiments, the composite biomarker score is calculated by adding to the composite biomarker score for each of the biomarkers that are met. In some embodiments, the composite biomarker score is calculated by subtracting from the composite biomarker score for each of certain biomarker scores that are met. In some embodiments, the composite biomarker score is calculated by adding to the composite biomarker score each of certain biomarkers that are met, and subtracting from the composite biomarker score for each of certain other biomarkers that are met. In some embodiments, the biomarkers that are met each increment the composite biomarker score. In some embodiments, the biomarkers that are met each increment the composite biomarker score by 1. In some embodiments, the biomarkers that are met increment the composite biomarker score by 2. In some embodiments, some of the biomarkers that are met increment the composite biomarker score by a lower value (such as by 1) and other biomarkers that are met increment the composite biomarker score by a higher value (such as by 2). In some embodiments, some of the biomarkers that are met increment the composite biomarker score by a lower value (such as by 1) if they are of a lower degree, and increment the composite biomarker score by a higher value (such as by 2) if they are of a higher degree. For example, the TMB score biomarker, in some embodiments, increments the composite biomarker score by a lower value (such as by 1) if the TMB score is a lower value (such as at least 10 and less than 20), but increments the composite biomarker score by a higher value (such as by 2) if the TMB score is a higher value (such as at least 20). In some embodiments, the biomarkers that are met do not increment the score (for example, if the particular biomarker is not included in the particular embodiment of the composite biomarker score). In some embodiments, the biomarkers that are not met increment the composite biomarker score (such as when the biomarker is a negative biomarker). In some embodiments, the biomarkers that are not met increment the score by 1. In some embodiments, at least some of the biomarkers that are met decrease the score (for example, if the particular individual biomarker is included in the particular embodiment of the composite biomarker score). In some embodiments, the biomarkers that are met decrease the composite biomarker score (such as when the biomarker is a negative biomarker). In some embodiments, a biomarker that is met decreases the score by 1. In various embodiments, the composite biomarker score may include individual biomarkers that increment the score by a positive value (positive individual biomarkers) and other individual biomarkers that decrease the score by a value (negative individual biomarkers). In addition, in some embodiments, the individual biomarkers may increment or decrease the score by different values. For example, in an exemplary embodiment, an individual biomarker may increment the score by 0 if below a certain value, by 1 if within a certain range, and by 2 if above said range.

In some embodiments, the composite biomarker score is an integer. In some embodiments, the composite biomarker score is not an integer. In some embodiments, the composite biomarker score starts at 0 and is incremented by each individual biomarker that is met. In some embodiments, the composite biomarker starts at 0 and is decreased by each of certain individual biomarkers that are met.

In some embodiments, the determination of whether the subject will likely benefit from the treatment comprising the immune checkpoint inhibitor is based on the composite biomarker score being at least a threshold value. The threshold value may, for example, represent a dividing line between a high likelihood of the subject responding to the immune checkpoint inhibitor and a lower likelihood (such as a medium likelihood, an indeterminate likelihood, or a low likelihood).

The composite biomarker score is determined based on at least some of the biomarkers disclosed herein. In some embodiments, the composite biomarker score is determined based on all of the biomarkers disclosed herein.

An individual biomarker may be considered “met” when the biomarker condition contributes to the composite biomarker score. For example, in an exemplary embodiment, if a tumor sample is assessed to have between 1% and less than 50% of cells positive for PD-L1 (such as by IHC), then the biomarker “the percent of cells in the tumor sample that are positive for PD-L1,” with the condition being at least 1% and less than 50%, is considered “met.” In some embodiments, all assessed biomarkers are met. In some embodiments, none of the assessed biomarkers are met. In some embodiments, some biomarkers are met and some biomarkers are not met. In some embodiments, the tumor mutational burden (TMB) score condition is met. In some embodiments, the TMB score condition is met and increments the composite biomarker score. In some embodiments, the TMB score condition is not met. In some embodiments, the TMB score condition is not met and does not increment the composite biomarker score. In some embodiments, the percent of cells in the tumor sample that are positive for PD-L1 condition is met. In some embodiments, the percent of cells in the tumor sample that are positive for PD-L1 condition is met and increments the composite biomarker score. In some embodiments, the percent of cells in the tumor sample that are positive for PD-L1 condition is not met. In some embodiments, the percent of cells in the tumor sample that are positive for PD-L1 condition is not met and does not increment the composite biomarker score. In some embodiments, the presence of a cancer-associated mutation in a ARID1A gene in the sample condition is met. In some embodiments, the presence of a cancer-associated mutation in a ARID1A gene in the sample condition is met and increments the composite biomarker score. In some embodiments, the presence of a cancer-associated mutation in a ARID1A gene in the sample condition is not met. In some embodiments, the presence of a cancer-associated mutation in a ARID1A gene in the sample condition is not met and does not increment the composite biomarker score. In some embodiments, the presence of a cancer-associated mutation in a KEAP1/NFE2L2 pathway gene in the sample condition is met. In some embodiments, the presence of a cancer-associated mutation in a KEAP1/NFE2L2 pathway gene in the sample condition is met and does not increment the composite biomarker score. In some embodiments, the presence of a cancer-associated mutation in a KEAP1/NFE2L2 pathway gene in the sample condition is met and increments the composite biomarker score. In some embodiments, the presence of a cancer-associated mutation in a KEAP1/NFE2L2 pathway gene in the sample condition is met and decreases the composite biomarker score. In some embodiments, the presence of a cancer-associated mutation in a KEAP1/NFE2L2 pathway gene in the sample condition is not met. In some embodiments, the presence of a cancer-associated mutation in a KEAP1/NFE2L2 pathway gene in the sample condition is not met and increments the composite biomarker score. In some embodiments, the presence of a cancer-associated mutation in a KEAP1/NFE2L2 pathway gene in the sample condition is not met and does not increment or decrease the composite biomarker score. In some embodiments, the presence of a cancer-associated mutation in a STK11/LKB1 gene in the sample condition is met. In some embodiments, the presence of a cancer-associated mutation in a STK11/LKB1 gene in the sample condition is met and does not increment the composite biomarker score. In some embodiments, the presence of a cancer-associated mutation in a STK11/LKB1 gene in the sample condition is met and decreases the composite biomarker score. In some embodiments, the presence of a cancer-associated mutation in a STK11/LKB1 gene in the sample condition is not met. In some embodiments, the presence of a cancer-associated mutation in a STK11/LKB1 gene in the sample condition is not met and increments the composite biomarker score. In some embodiments, the presence of a cancer-associated mutation in a STK11/LKB1 pathway gene in the sample condition is not met and does not increment or decrease the composite biomarker score. In some embodiments, the presence of a cancer-associated mutation in a CDKN2A in the sample condition is met. In some embodiments, the presence of a cancer-associated mutation in a CDKN2A gene in the sample condition is met and does not increment the composite biomarker score. In some embodiments, the presence of a cancer-associated mutation in a CDKN2A gene in the sample condition is met and increments the composite biomarker score. In some embodiments, the presence of a cancer-associated mutation in a CDKN2A gene in the sample condition is met and decreases the composite biomarker score. In some embodiments, the presence of a cancer-associated mutation in a CDKN2A gene in the sample condition is not met. In some embodiments, the presence of a cancer-associated mutation in a CDKN2A gene in the sample condition is not met and increments the composite biomarker score. In some embodiments, the presence of a cancer-associated mutation in a CDKN2A gene in the sample condition is not met and does not increment the composite biomarker score. In some embodiments, the cancer-associated mutation in a CDKN2A gene is a deletion. In some embodiments, the cancer-associated mutation in a CDKN2A gene is a deletion in both copies of CDKN2A (e.g., a homozygous deletion for CDKN2A). In some embodiments, the presence of a cancer-associated mutation in a DDR gene in the sample condition is met. In some embodiments, the presence of a cancer-associated mutation in one or more DDR genes in the sample condition is met. In some embodiments, the presence of a cancer-associated mutation in a DDR gene in the sample condition is met and increments the composite biomarker score. In some embodiments, the presence of a cancer-associated mutation in one or more DDR genes in the sample condition is met and increments the composite biomarker score. In some embodiments, the presence of a cancer-associated mutation in a DDR gene in the sample condition is not met. In some embodiments, the presence of a cancer-associated mutation in one or more DDR genes in the sample condition is not met. In some embodiments, the presence of a cancer-associated mutation in a DDR gene in the sample condition is not met and does not increment the composite biomarker score. In some embodiments, the presence of a cancer-associated mutation in one or more DDR genes in the sample condition is not met and does not increment the composite biomarker score. In some embodiments, the at least one DDR gene is at least one of ATM, BRCA2, BRIP1, MRE11, POLE, MSH2, PARP1, MLH1, MSH6, PMS2, BRCA1, PALB2, and/or BARD1. In some embodiments, the at least one DDR gene includes each of ATM, BRCA2, BRIP1, MRE11, POLE, MSH2, PARP1, MLH1, MSH6, PMS2, BRCA1, PALB2, and/or BARD1.

In some embodiments, the tumor mutational burden (TMB) score condition is met. In some embodiments, the TMB score condition is met and increments the composite biomarker score. In some embodiments, the TMB score condition is met and increments the composite biomarker score by 1. In some embodiments, the TMB score condition is met and increments the composite biomarker score by 2. In some embodiments, the TMB score condition is not met. In some embodiments, the TMB score condition is not met and does not increment the composite biomarker score. In some embodiments, the TMB score condition is not met and increments the biomarker score by 0. In some embodiments, the percent of cells in the tumor sample that are positive for PD-L1 condition is met. In some embodiments, the percent of cells in the tumor sample that are positive for PD-L1 condition is met and increments the composite biomarker score by 1. In some embodiments, the percent of cells in the tumor sample that are positive for PD-L1 condition is met and increments the composite biomarker score by 2. In some embodiments, the percent of cells in the tumor sample that are positive for PD-L1 condition is not met. In some embodiments, the percent of cells in the tumor sample that are positive for PD-L1 condition is not met and does not increment the composite biomarker score. In some embodiments, the percent of cells in the tumor sample that are positive for PD-L1 condition is not met and increments the composite biomarker score by 0. In some embodiments, the presence of a cancer-associated mutation in a ARID1A gene in the sample condition is met. In some embodiments, the presence of a cancer-associated mutation in a ARID1A gene in the sample condition is met and increments the composite biomarker score. In some embodiments, the presence of a cancer-associated mutation in a ARID1A gene in the sample condition is met and increments the composite biomarker score by 1. In some embodiments, the presence of a cancer-associated mutation in a ARID1A gene in the sample condition is not met. In some embodiments, the presence of a cancer-associated mutation in a ARID1A gene in the sample condition is not met and does not increment the composite biomarker score. In some embodiments, the presence of a cancer-associated mutation in a ARID1A gene in the sample condition is not met and increments the composite biomarker score by 0. In some embodiments, the presence of a cancer-associated mutation in a KEAP1/NFE2L2 pathway gene in the sample condition is met. In some embodiments, the presence of a cancer-associated mutation in a KEAP1/NFE2L2 pathway gene in the sample condition is met and does not increment the composite biomarker score. In some embodiments, the presence of a cancer-associated mutation in a KEAP1/NFE2L2 pathway gene in the sample condition is met and increments the composite biomarker score by 0. In some embodiments, the presence of a cancer-associated mutation in a KEAP1/NFE2L2 pathway gene in the sample condition is met and decreases the composite biomarker score by 1. In some embodiments, the presence of a cancer-associated mutation in a KEAP1/NFE2L2 pathway gene in the sample condition is not met. In some embodiments, the presence of a cancer-associated mutation in a KEAP1/NFE2L2 pathway gene in the sample condition is not met and increments the composite biomarker score. In some embodiments, the presence of a cancer-associated mutation in a KEAP1/NFE2L2 pathway gene in the sample condition is not met and increments the composite biomarker score by 1. In some embodiments, the presence of a cancer-associated mutation in a KEAP1/NFE2L2 pathway gene in the sample condition is not met and does not increment the composite biomarker score. In some embodiments, the presence of a cancer-associated mutation in a STK11/LKB1 gene in the sample condition is met. In some embodiments, the presence of a cancer-associated mutation in a STK11/LKB1 gene in the sample condition is met and does not increment the composite biomarker score. In some embodiments, the presence of a cancer-associated mutation in a STK11/LKB1 gene in the sample condition is met and increments the composite biomarker score by 0. In some embodiments, the presence of a cancer-associated mutation in a STK11/LKB1 gene in the sample condition is met and decreases the composite biomarker score by 1. In some embodiments, the presence of a cancer-associated mutation in a STK11/LKB1 gene in the sample condition is not met. In some embodiments, the presence of a cancer-associated mutation in a STK11/LKB1 gene in the sample condition is not met and increments the composite biomarker score. In some embodiments, the presence of a cancer-associated mutation in a STK11/LKB1 gene in the sample condition is not met and increments the composite biomarker score by 1. In some embodiments, the presence of a cancer-associated mutation in a STK11/LKB1 gene in the sample condition is not met and does not increment the composite biomarker score. In some embodiments, the presence of a cancer-associated mutation in a CDKN2A in the sample condition is met. In some embodiments, the presence of a cancer-associated mutation in a CDKN2A gene in the sample condition is met and does not increment the composite biomarker score. In some embodiments, the presence of a cancer-associated mutation in a CDKN2A gene in the sample condition is met and increments the composite biomarker score by 0. In some embodiments, the presence of a cancer-associated mutation in a CDKN2A gene in the sample condition is met and decreases the composite biomarker score by 1. In some embodiments, the presence of a cancer-associated mutation in a CDKN2A gene in the sample condition is not met. In some embodiments, the presence of a cancer-associated mutation in a CDKN2A gene in the sample condition is not met and increments the composite biomarker score. In some embodiments, the presence of a cancer-associated mutation in a CDKN2A gene in the sample condition is not met and increments the composite biomarker score by 1. In some embodiments, the presence of a cancer-associated mutation in a CDKN2A gene in the sample condition is not met and does not increment the composite biomarker score by 1. In some embodiments, the cancer-associated mutation in a CDKN2A gene is a deletion. In some embodiments, the deletion is a homozygous deletion. In some embodiments, the presence of a cancer-associated mutation in a DDR gene in the sample condition is met. In some embodiments, the presence of a cancer-associated mutation in one or more DDR genes in the sample condition is met. In some embodiments, the presence of a cancer-associated mutation in a DDR gene in the sample condition is met and increments the composite biomarker score. In some embodiments, the presence of a cancer-associated mutation in a DDR gene in the sample condition is met and increments the composite biomarker score by 1. In some embodiments, the presence of a cancer-associated mutation in one or more DDR genes in the sample condition is met and increments the composite biomarker score. In some embodiments, the presence of a cancer-associated mutation in one or more DDR genes in the sample condition is met and increments the composite biomarker score by 1. In some embodiments, the presence of a cancer-associated mutation in a DDR gene in the sample condition is not met. In some embodiments, the presence of a cancer-associated mutation in one or more DDR genes in the sample condition is not met. In some embodiments, the presence of a cancer-associated mutation in a DDR gene in the sample condition is not met and does not increment the composite biomarker score. In some embodiments, the presence of a cancer-associated mutation in a DDR gene in the sample condition is not met and increments the biomarker score by 0. In some embodiments, the presence of a cancer-associated mutation in one or more DDR genes in the sample condition is not met and does not increment the composite biomarker score. In some embodiments, the presence of a cancer-associated mutation in one or more DDR genes in the sample condition is not met and increments the biomarker score by 0. In some embodiments, the at least one DDR gene is at least one of ATM, BRCA2, BRIP1, MRE11, POLE, MSH2, PARP1, MLH1, MSH6, PMS2, BRCA1, PALB2, and BARD1. In some embodiments, the at least one DDR gene includes each of ATM, BRCA2, BRIP1, MRE11, POLE, MSH2, PARP1, MLH1, MSH6, PMS2, BRCA1, PALB2, and BARD1.

Further disclosed herein are methods comprising creating a record associated with the subject designating the subject as (i) one who will likely benefit from the treatment comprising an immune checkpoint inhibitor or (ii) one who will likely not benefit from the treatment comprising an immune checkpoint inhibitor, wherein the designation is based on the determined composite biomarker score described herein. In some embodiments, the methods disclosed herein further comprise administering the immune checkpoint inhibitor to the identified subject. In some embodiments, the cancer is a lung cancer, a melanoma, a bladder cancer, or a head and neck cancer.

In some embodiments, the immune checkpoint inhibitor is a PD-1 inhibitor, such as one or more of nivolumab, pembrolizumab, cemiplimab, and/or dostarlimab. In some embodiments, the immune checkpoint inhibitor is a PD-L1 inhibitor. In some embodiments, the PDL-1 inhibitor is atezolizumab, avelumab, or durvalumab. In some embodiments, the immune checkpoint inhibitor is a CTLA-4 inhibitor. In some embodiments, the CTLA-4 inhibitor is ipilimumab.

Disclosed herein are methods comprising determining a composite biomarker score; wherein the composite biomarker score is determined based, at least in part, on assessed biomarkers in a tumor sample. In some embodiments, the tumor sample is a tissue biopsy sample or a liquid biopsy sample. In some embodiments, the sample is a tissue biopsy and comprises a tumor biopsy or tumor specimen. In some embodiments, the sample is a liquid biopsy sample and comprises blood, circulating tumor cells, serum, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, cell-free RNA from the cancer, or any combination thereof. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or both.

Disclosed herein are methods that treat a subject having a cancer, wherein the subject is identified as one who will benefit from an immune checkpoint inhibitor according to any of the methods described herein, the method comprising administering the immune checkpoint inhibitor to the subject.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### A. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### B. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

Throughout this application, various parameter values may be presented in a range format. It should be understood that the description in range format is merely for convenience and brevity, and should not be construed as an inflexible limitation on the scope of the disclosure. Accordingly, the description of a range should be considered to have specifically disclosed all possible subranges as well as individual numerical values within that range, irrespective of whether a specific numerical value or specific sub-range is expressly stated. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 1.4, 2, 3, 3.6, 4, 5, 5.8, and 6. This applies regardless of the breadth of the range.

Numbers may be expressed herein as being “about” a particular value. Similarly, ranges may be expressed herein as from “about” one particular value and/or to “about” another particular value. The terms “about” and “approximately” shall generally mean an acceptable degree of error or variation for a given value or range of values, such as, for example, a degree of error or variation that is within 20 percent (%), within 15%, within 10%, or within 5% of a given value or range of values.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The term “detection” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy, e.g., a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations, or other mutations or alterations), polypeptides, polypeptide and polynucleotide modifications (e.g., post-translational modifications), carbohydrates, and/or glycolipid-based molecular markers.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

An “anti-cancer agent” or “anti-cancer treatment” can refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

The term “inactivating mutation” refers to any mutation in a nucleic acid or protein that results in reduced expression and/or activity of the protein.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

“Alteration” or “altered structure” as used herein, of a gene or gene product (e.g., a marker gene or gene product) refers to the presence of a mutation or mutations within the gene or gene product, e.g., a mutation, which affects integrity, sequence, structure, amount or activity of the gene or gene product, as compared to the normal or wild-type gene. The alteration can be in amount, structure, and/or activity in a cancer tissue or cancer cell, as compared to its amount, structure, and/or activity, in a normal or healthy tissue or cell (such as a control), and is associated with a disease state, such as cancer. For example, an alteration which is associated with cancer, or predictive of responsiveness to anti-cancer therapeutics, can have an altered nucleotide sequence (such as a mutation), amino acid sequence, chromosomal translocation, intra-chromosomal inversion, copy number, expression level, protein level, protein activity, epigenetic modification (e.g., methylation or acetylation status), or post-translational modification, in a cancer tissue or cancer cell, as compared to a normal, healthy tissue or cell. Exemplary mutations include, but are not limited to, point mutations (e.g., silent, missense, or nonsense), deletions, insertions, inversions, duplications, amplification, translocations, inter- and intra-chromosomal rearrangements. Mutations can be present in the coding or non-coding region of the gene. In certain embodiments, the alteration(s) is detected as a rearrangement, e.g., a genomic rearrangement comprising one or more introns or fragments thereof (e.g., one or more rearrangements in the 5′- and/or 3′-UTR). In certain embodiments, the alterations are associated (or not associated) with a phenotype, e.g., a cancerous phenotype (e.g., one or more of cancer risk, cancer progression, cancer treatment or resistance to cancer treatment). In one embodiment, the alteration (or tumor mutational burden) is associated with one or more of: a genetic risk factor for cancer, a positive treatment response predictor, a negative treatment response predictor, a positive prognostic factor, a negative prognostic factor, or a diagnostic factor.

“Likely to” or “increased likelihood,” as used herein, refers to an increased probability that an item, object, thing or person will occur. Thus, in one example, a subject that is likely to respond to treatment has an increased probability of responding to treatment relative to a reference subject or group of subjects.

“Unlikely to” refers to a decreased probability that an event, item, object, thing or person will occur with respect to a reference. Thus, a subject that is unlikely to respond to treatment has a decreased probability of responding to treatment relative to a reference subject or group of subjects.

“Or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

“Variant,” as used herein, refers to a structure that can be present at a subgenomic interval that can have more than one structure, e.g., an allele at a polymorphic locus.

It should be recognized that use of ordinal terms such as “first” and “second” in the description of methods and systems disclosed herein does not by itself connote any priority, order of importance of one system component over another, or the temporal order in which acts of a method are performed, but are used merely as labels to distinguish, for example, one system component having a certain name from another system component having the same name but for the use of the ordinal term to distinguish the two system components.

Additionally, various implementations of the methods and systems set forth herein may be described in terms of exemplary block diagrams, process flow charts, and other illustrations. As will become apparent to one of ordinary skill in the art after reading this document, the various implementations set forth herein can be implemented without confinement to the illustrated examples. For example, block diagrams and their accompanying description should not be construed as mandating a particular architecture or configuration. Similarly, in exemplary process flow charts, some blocks are optionally combined, the order of some blocks is optionally changed, and some blocks are optionally omitted. In some implementations, additional steps may be performed in combination with the exemplary processes. Accordingly, the methods and systems as described and illustrated in greater detail below are exemplary by nature and, as such, should not be viewed as limiting.

### C. SUBJECTS TO BE TREATED, ASSESSED, AND/OR IDENTIFIED

The methods of the present disclosure pertain, in various embodiments, to subjects with a cancer and/or samples (such as tumor biopsy samples and/or liquid biopsy samples) obtained from a subject with a cancer. In some embodiments, the methods pertain to identifying a subject with a cancer who will likely benefit from a treatment comprising an immune checkpoint inhibitor, the method comprising determining a composite biomarker score; wherein the composite biomarker score is determined based on at least three of the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

In some embodiments, the methods pertain to predicting if a subject with a cancer will likely benefit from a treatment comprising an immune checkpoint inhibitor, the method comprising determining a composite biomarker score; wherein the composite biomarker score is determined based on the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor.

In some embodiments, the methods pertain to stratifying a subject with a cancer for treatment comprising an immune checkpoint inhibitor, the method comprising determining a composite biomarker score; wherein the composite biomarker score is determined based on the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample; and determining, based on the determined composite biomarker score, whether the subject will likely benefit from the treatment comprising the immune checkpoint inhibitor. In some embodiments, the methods pertain to treating a subject having a cancer, wherein the subject is identified as one who will benefit from an immune checkpoint inhibitor according to any of the methods described herein, wherein the methods comprise administering the immune checkpoint inhibitor to the subject.

In the various embodiments described herein, the subject has or had a cancer. In some embodiments, the subject may have had or may have a cancer. In some embodiments, the subject has been, or is being treated, for the cancer. In some embodiments, the subject is in need of being monitored for cancer progression or regression, e.g., after being treated with a cancer therapy. In some embodiments, the subject is in need of being monitored for relapse of cancer. In some embodiments, the subject is suspected of having cancer. In some embodiments, the subject is being tested for cancer. In some embodiments, the subject has a genetic predisposition to a cancer (e.g., having a mutation that increases his or her baseline risk for developing a cancer). In some embodiments, the subject is in need of a first line treatment for the cancer.

In certain embodiments, the sample is from a subject having a cancer. Exemplary cancers include, but are not limited to, B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, a carcinoid tumor, or a gastro-esophageal tumor. In some embodiments, the cancer is a lung cancer, a melanoma, a bladder cancer, head and neck cancer, or a gastro-esophageal tumor. In some embodiments, the cancer is a lung cancer. In some embodiments, the cancer is non-small cell lung carcinoma (NSCLC). In some embodiments, the cancer is squamous non-small cell lung carcinoma. In some embodiments, the cancer is non-squamous non-small cell lung carcinoma. In some embodiments, the patients are wild-type (e.g. do not possess) a known or likely pathogenic driver mutations in the gene encoding for epidermal growth factor receptor (EGFR) and anaplastic lymphoma kinase (ALK). In some embodiments, the patients are wild-type (e.g. do not possess) a known or likely pathogenic driver mutations in the genes encoding for B-Raf (BRAF), mesenchymal epithelial factor (MET), receptor tyrosine kinase (ROS1), and rearranged during transfection (RET).

In certain embodiments, the sample is from an individual having a solid tumor, a hematological cancer, or a metastatic form thereof. In certain embodiments, the sample is obtained from a subject having a cancer. In certain embodiments, the sample is obtained from a subject who has not received a therapy to treat a cancer, is receiving a therapy to treat a cancer, or has received a therapy to treat a cancer, as described herein.

In some embodiments, the subject has been previously treated with an anti-cancer therapy, e.g., one or more anti-cancer therapies (e.g. any of the anti-cancer therapies of the disclosure). For example, the sample may be from subject that has been treated with an anti-cancer therapy comprising one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, or a cytotoxic agent. In some embodiments, for a subject who has been previously treated with an anti-cancer therapy, a post-anti-cancer therapy sample, e.g., specimen is obtained, e.g., collected. In some embodiments, the post-anti-cancer therapy sample is a sample obtained, e.g., collected, after the completion of the targeted therapy. In some embodiments, for a subject who has been previously treated with an anti-cancer therapy, the subject is considered a responder or a non-responder. In some embodiments, the subject has previously been treated with a chemotherapy. In some embodiments, the chemotherapy is a platinum therapy.

In some embodiments, the subject has not previously received, or is not currently receiving, a treatment for the cancer. In some embodiments, the subject has not previously received a regimen of chemotherapy. In some embodiments, the subject has not received a chemotherapy for the cancer.

In some embodiments, the subject has previously received, or is currently receiving, a treatment for the cancer. In some embodiments, the subject has previously received a regimen of chemotherapy. In some embodiments, the subject has previously received a chemotherapy for the cancer. In some embodiments, the chemotherapy is a platinum-based therapy.

In some embodiments, the individual is a human. In some embodiments, the individual is a non-human mammal.

### D. SAMPLING

The methods of the present disclosure also involve, in certain embodiments, determining or assessing one or more features of a sample (such as a tumor sample) or samples obtained from an individual having a cancer. For example, to determine the composite biomarker score, the methods may comprise obtaining or receiving a tumor sample from the subject and then assessing one or more biomarkers in the tumor sample for use in calculating the composite biomarker score. In some embodiments, the sample is associated with the cancer to be treated or assessed. In some embodiments, the sample is from a solid tumor (e.g., a tumor biopsy sample) of the cancer. In some embodiments, the sample is from a liquid sample (e.g., a liquid biopsy sample) of the cancer. Samples may be analyzed by methods well-known to those skilled in the art. Non-limiting examples include sequencing based methods, immunohistochemistry, and immunoassays.

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some embodiments, the one or more bait molecules each comprise a capture moiety. In some embodiments, the capture moiety is biotin. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof. In some embodiments, the sample is a tissue biopsy and comprises a tumor biopsy or tumor specimen. In some embodiments, the sample is a liquid biopsy sample and comprises blood, circulating tumor cells, serum, plasma, cerebrospinal fluid, sputum, stool, urine, or saliva. In some embodiments, the sample comprises cells and/or nucleic acids from the cancer.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some embodiments, the sample comprises mRNA, DNA, circulating tumor DNA (ctDNA), cell-free DNA, cell-free RNA from the cancer, or any combination thereof. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or both. In some embodiments, the individual is a human.

In some embodiments, the method comprises determining the composite biomarker score, wherein the composite biomarker score is determined on the following: (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene in the sample. In some embodiments, the method is also determined by the status of one or more human leukocyte antigen (HLA) genes of the cancer and has been determined to exhibit loss of heterozygosity (LOH).

### E. METHODS FOR DETERMINING THE COMPOSITE BIOMARKER SCORE

In some embodiments, the methods described herein comprise determining a composite biomarker score. The composite biomarker score integrates a plurality of individual biomarkers to give a more accurate and precise prediction of a subject's response to an immune checkpoint inhibitor therapy. In a preferred embodiment, a plurality of biomarkers are assessed for meeting particular conditions (for example, the percent of cells in a tumor sample that are positive for PD-L1 are assessed to determine if the percent of cells positive for PD-L1 is at least 1% and less than 50%). If a condition is met for a particular biomarker, the composite biomarker score is increased (i.e., incremented) in this preferred embodiment. Once the composite biomarker score is calculated, it is then useful, for example, for selecting a treatment plan for the subject or for preparing a prognostic report for the subject (such as to advise the subject or the subject's caregiver which treatments the subject may respond to). For example, if the composite biomarker score is at least a threshold value, the subject may be assessed as likely to benefit from treatment comprising an immune checkpoint inhibitor. In contrast, if the composite biomarker score is not at least a threshold value, the subject may be assessed as unlikely to benefit from treatment comprising an immune checkpoint inhibitor (ICI), or may be indicated for treatment with a combination therapy (such as an immune checkpoint inhibitor and an additional anti-cancer agent) or for a non-ICI therapy. Intermediate or indeterminate or medium composite biomarker scores may also be obtained (such as a medium likelihood, representing a likelihood in between a low likelihood and a high likelihood). In some embodiments, the composite biomarker score is determined based on, at least in part, a tumor mutational burden (TMB) score. In some embodiments, the composite biomarker score is determined based on, at least in part, the percent of cells in a tumor sample that are positive for PD-L1. In some embodiments, the composite biomarker score is determined based on, at least in part, the presence of a cancer-associated mutation in a ARID1A gene in a tumor sample. In some embodiments, the composite biomarker score is determined based on, at least in part, the presence of a cancer-associated mutation in a KEAP1/NFE2L2 pathway gene in the sample. In some embodiments, the composite biomarker score is determined based on, at least in part, the presence of a cancer-associated mutation in a STK11/LKB1 gene in a tumor sample. In some embodiments, the composite biomarker score is determined based on, at least in part, the presence of a cancer-associated mutation in a CDKN2A gene in a tumor sample. In some embodiments, the composite biomarker score is determined based on, at least in part, the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in a tumor sample.

In some embodiments, the disclosure provides for a method of identifying a subject with a cancer who will likely benefit from a treatment comprising an immune checkpoint inhibitor, the method comprising determining a composite biomarker score. In some embodiments, the composite biomarker score is determined based on, at least in part, a tumor mutational burden (TMB) score. In some embodiments, the composite biomarker score is determined based on, at least in part, the percent of cells in a tumor sample that are positive for PD-L1. In some embodiments, the composite biomarker score is determined based on, at least in part, the presence of a cancer-associated mutation in a ARID1A gene in a tumor sample. In some embodiments, the composite biomarker score is determined based on, at least in part, the presence of a cancer-associated mutation in a KEAP1/NFE2L2 pathway gene in the sample. In some embodiments, the composite biomarker score is determined based on, at least in part, the presence of a cancer-associated mutation in a STK11/LKB1 gene in a tumor sample. In some embodiments, the composite biomarker score is determined based on, at least in part, the presence of a cancer-associated mutation in a CDKN2A gene in a tumor sample. In some embodiments, the composite biomarker score is determined based on, at least in part, the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in a tumor sample.

In some embodiments, the disclosure provides for a method of predicting if a subject with cancer will likely benefit from a treatment comprising an immune checkpoint inhibitor, the method comprising a composite biomarker score. In some embodiments, the composite biomarker score is determined based on, at least in part, a tumor mutational burden (TMB) score. In some embodiments, the composite biomarker score is determined based on, at least in part, the percent of cells in a tumor sample that are positive for PD-L1. In some embodiments, the composite biomarker score is determined based on, at least in part, the presence of a cancer-associated mutation in a ARID1A gene in a tumor sample. In some embodiments, the composite biomarker score is determined based on, at least in part, the presence of a cancer-associated mutation in a KEAP1/NFE2L2 pathway gene in the sample. In some embodiments, the composite biomarker score is determined based on, at least in part, the presence of a cancer-associated mutation in a STK11/LKB1 gene in a tumor sample. In some embodiments, the composite biomarker score is determined based on, at least in part, the presence of a cancer-associated mutation in a CDKN2A gene in a tumor sample. In some embodiments, the composite biomarker score is determined based on, at least in part, the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in a tumor sample.

In some embodiments, the disclosure provides for a method of stratifying a subject with a cancer for treatment comprising an immune checkpoint inhibitor, the method comprising determining a composite biomarker score. In some embodiments, the composite biomarker score is determined based on, at least in part, a tumor mutational burden (TMB) score. In some embodiments, the composite biomarker score is determined based on, at least in part, the percent of cells in a tumor sample that are positive for PD-L1. In some embodiments, the composite biomarker score is determined based on, at least in part, the presence of a cancer-associated mutation in a ARID1A gene in a tumor sample. In some embodiments, the composite biomarker score is determined based on, at least in part, the presence of a cancer-associated mutation in a KEAP1/NFE2L2 pathway gene in the sample. In some embodiments, the composite biomarker score is determined based on, at least in part, the presence of a cancer-associated mutation in a STK11/LKB1 gene in a tumor sample. In some embodiments, the composite biomarker score is determined based on, at least in part, the presence of a cancer-associated mutation in a CDKN2A gene in a tumor sample. In some embodiments, the composite biomarker score is determined based on, at least in part, the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in a tumor sample.

In some embodiments, the disclosure provides a method of treating a subject having a cancer, wherein the subject is identified as one who will benefit from an immune checkpoint inhibitor according to any of the methods disclosed within, the method comprising administering the immune checkpoint inhibitor to the subject.

The methods of the present disclosure also comprise determining a composite biomarker score for a subject with a cancer, wherein the composite biomarker score is based on at least three or more of the biomarkers disclosed herein. In some embodiments, the composite biomarker score is based on at least four or more of the biomarkers disclosed herein. In some embodiments, the composite biomarker score is based on at least five or more of the biomarkers disclosed herein. In some embodiments, the composite biomarker score is based on at least six or more of the biomarkers disclosed herein. In some embodiments, the composite biomarker score is based on at least seven or more of the biomarkers disclosed herein. The biomarker score is determined based on a tumor sample associated with the cancer. In some embodiments, the composite biomarker score is determined based on one or more tumor samples associated with the cancer. In some embodiments, the composite biomarker score is determined based on sampling one or more tumor samples associated with the cancer.

### F. INDIVIDUAL BIOMARKERS

As used herein, the term “biomarker” refers to a measurable indicator of a subject having a cancer which may be evaluated. The individual biomarkers disclosed in the present application are selected as inputs of a composite biomarker score, which is described herein. The individual biomarker assessed may be a protein (such as the percent of cells positive for PD-L1) or a nucleic acid (such as the presence or absence of certain mutations, or the loss of heterozygosity in a genomic sample). A biomarker may be genetic (e.g. inherited) or not-inherited (e.g. tumor markers (and mutations) that are not inherited, and for non-genomic markers, such as PD-L1 protein expression on the tumor as well as the tumor mutational burden). Certain aspects of the methods provided herein relate to acquiring knowledge of, detecting expression levels, or detecting one or more mutations in genes or nucleic acids. The biomarkers of this present disclosure are used to determine a composite biomarker score.

### 1. Tumor Mutational ARDEN

In some embodiments, the methods described herein relate to detection of the tumor mutational burden score of a tumor sample. Tumor mutational burden (TMB) is, broadly, the number of somatic mutations per megabase (i.e., mutations/MB or mut/MB) of a genomic region. A TMB score identifies the number of genetic changes in the cancer. It has been discovered that a TMB score (which can be expressed, for example, as mutations/MB) can inform treatment decisions, including, in some embodiments, whether to administer an immune checkpoint inhibitor therapy to a patient if a TMB score is at least a threshold TMB score. The TMB score can be determined using a variety of next-generation sequencing (NGS) techniques.

In some embodiments, the methods of the disclosure comprise determining a tumor mutational burden (TMB) score in a sample. In some embodiments, the TMB score is a blood TMB (bTMB) score. In some embodiments, the TMB score is a tissue TMB score (tTMB score) or a solid tumor TMB score.

In some embodiments, the TMB score is determined by sequencing. In some embodiments, the TMB score is determined by sequencing using a high-throughput sequencing technique, such as next-generation sequencing (NGS), an NGS-based method, or an NGS-derived method. In some embodiments, the NGS method is selected from whole genome sequencing (WGS), whole exome sequencing (WES) or a comprehensive genomic profiling (CGP). In some embodiments, the sequencing comprises sequencing a panel of cancer genes. In some embodiments, the TMB score reflects the number of nonsynonymous mutations, such as missense mutations or nonsense mutations, in a sequence. In some embodiments, the TMB score is determined by normalizing a matched tumor biopsy sample sequence with germline sequences to exclude inherited germline mutations.

The “threshold TMB score” described herein refers to a predetermined TMB score which a measured TMB score (i.e., the TMB score determined in a sample from an individual having a cancer) is compared with. Comparison of the determined TMB score with the threshold TMB score is used, in certain embodiments, to inform treatment decisions or identify treatment options for an individual. For example, the threshold TMB score may be a condition which, if the TMB score is at least the threshold TMB score, is met as part of determining the composite biomarker score described herein.

As used herein, the terms “solid tumor TMB score,” “tissue TMB score,” and “tTMB score,” are used interchangeably and refers to a numerical value that reflects the number of somatic mutations detected in a solid tumor biopsy sample (e.g., a solid tumor biopsy sample) obtained from a subject (e.g., a subject having a cancer). Tumor mutational burden is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety. In some embodiments, tumor mutational burden is measured according to the methods described in Montesion, M., et al., Cancer Discovery (2021) 11(2):282-92. In some embodiments, tumor mutational burden is measured according to the methods described in Chalmers et al., “Analysis of 100,00 human cancer genomes reveals the landscape of tumor mutational burden,” Genome Med. 2017; 9(1):34).

In some embodiments, tumor mutational burden is assessed based on the number of non-driver somatic coding mutations/megabase (mut/MB) of genome sequenced.

In some embodiments, the TMB score is less than a threshold value of mutations/Mb. In some embodiments, the TMB score meets a threshold value of mutations/Mb. In some embodiments, the TMB score meets a threshold value of mutations/Mb and the composite biomarker score is incremented. In some embodiments, the threshold value is at least 10 mutations/Mb. In some embodiments, the TMB score is at least about 20 mut/Mb. In some embodiments, the threshold value is met and the composite biomarker score is incremented, and a second threshold value is met and the composite biomarker score is further incremented. In some embodiments, the threshold value is 10 mut/Mb. In some embodiments, the second threshold value is 20 mut/Mb.

In some embodiments, the TMB score is at least 10 mut/MB, and the composite biomarker score is incremented. In some embodiments, the TMB score is at least 10 mut/Mb and no more than 20 mut/Mb, and the composite biomarker score is incremented. In some embodiments, the TMB score is at least 10 mut/Mb, at least 11 mut/Mb, at least 12 mut/Mb, at least 13 mut/Mb, at least 14 mut/Mb, at least 15 mut/Mb, at least 16 mut/Mb, at least 17 mut/Mb, at least 18 mut/Mb, at least 19 mut/Mb and no more than 20 mut/Mb, and the composite biomarker score is incremented. In some embodiments, the TMB score is 20 mut/Mb, and the composite biomarker score is incremented. In some embodiments, the TMB score is at least 20 mut/Mb, and the composite biomarker score is incremented. In some embodiments, the TMB score is less than 10 mut/Mb and the composite biomarker score is not incremented. In some embodiments, the TMB score is less than 10 mut/Mb and the composite biomarker score is incremented by 0. In some embodiments, the composite biomarker score is incremented if the TMB score of at least 10 is met and further incremented if the TMB score of at least 20 is met. In some embodiments, the TMB score is determined based on between about 100 kb to about 10 Mb of genomic sequence. In some embodiments, the TMB score is determined based on between about 0.8 Mb to about 1.1 Mb of genomic sequence. In some embodiments, the TMB score is used to determine in part a composite biomarker score.

In some embodiments, the TMB score is less than about 10 mutations/Mb. In some embodiments, the TMB score is at least 10 mutations/Mb to less than about 20 mutations/Mb. In some embodiments, the TMB score is at least 10 mutations/Mb. In some embodiments, the TMB score is at least about 20 mut/Mb.

In some embodiments, the TMB score is at least 10 mut/Mb, and the composite biomarker score is incremented. In some embodiments, the TMB score is at least 10 mut/Mb and no more than 20 mut/Mb, and the composite biomarker score is incremented. In some embodiments, the TMB score is at least 20 mut/Mb, and the composite biomarker score is incremented. In some embodiments, the TMB score is less than 10 mut/Mb and the composite biomarker score is not incremented. In some embodiments, the composite biomarker score is incremented if the TMB score of at least 10 is met and further incremented if the TMB score of at least 20 is met. In some embodiments, the TMB score is determined based on between about 100 kb to about 10 Mb of genomic sequence. In some embodiments, the TMB score is determined based on between about 0.8 Mb to about 1.1 Mb of genomic sequence. In some embodiments, the TMB score is used to determine, in part, the composite biomarker score.

In some embodiments, the TMB score is at least 10 mut/Mb, and the composite biomarker score is incremented by one. In some embodiments, the TMB score is at least 10 mut/Mb and no more than 20 mut/Mb, and the composite biomarker score is incremented by one. In some embodiments, the TMB score is at least 20 mut/Mb, and the composite biomarker score is incremented by two. In some embodiments, the TMB score is less than 10 mut/Mb and the composite biomarker score is not incremented. In some embodiments, the composite biomarker score is incremented by 1 if the TMB score of at least 10 is met and incremented by 2 if the TMB score of at least 20 is met. In some embodiments, the TMB score is determined based on between about 100 kb to about 10 Mb of genomic sequence. In some embodiments, the TMB score is determined based on between about 0.8 Mb to about 1.1 Mb of genomic sequence.

In some embodiments, the composite biomarker score is determined based on, at least in part, the TMB score. In some embodiments, a plurality of threshold values are defined, wherein the composite biomarker score is incremented based on each threshold met. In an exemplary embodiment, two thresholds are defined of TMB score of at least 10 and TMB score of at least 20. In this exemplary embodiment, if the assessed TMB score for a tumor sample is less than 10, then the composite biomarker score is not incremented; if the TMB score is at least 10, then the composite biomarker score is incremented (for example, by 1); if the TMB score is at least 20, then the composite biomarker score is incremented by a greater amount (for example, by 2). In this example, an increasing TMB score represents an increased likelihood that subject is a candidate for treatment with an immune checkpoint inhibitor, and therefore higher TMB scores may be weighted to increment the composite biomarker score by a greater amount than lower TMB scores.

### 2 PD-L1-Expression

The composite biomarker score may be based, at least in part, on the percent of cells in a tumor sample that are positive for PD-L1. When more cells in the tumor sample are found to express PD-L1, the composite biomarker score may be incremented, representing an increased likelihood that the subject will respond to immune checkpoint inhibitor therapy. In some embodiments, the methods provided herein comprise determining the percent of cells in the tumor sample that are positive for PD-L1. In some embodiments, the percent of cells in the tumor sample that are positive for PD-L1 comprises measuring the level of PD-L1 expression in a sample, e.g., in a sample from a tumor obtained from a subject. In some embodiments, the methods comprise determining the percent of cells in the tumor sample that are positive for PD-L1 and treating a subject having a cancer, wherein the subject is identified as one who will benefit from an immune checkpoint inhibitor according to any of the methods disclosed herein, the method comprising administering the immune checkpoint inhibitor to the subject.

In some embodiments, the level of PD-L1 protein and/or PD-L1 mRNA is assessed in sample from a subject, such as a sample described herein. Any suitable method for measuring PD-L1 expression in a sample from a subject may be used. For example, in some embodiments, the percent of cells in the tumor sample that are positive for PD-L1 is measured based on the level of PD-L1 mRNA in the sample. Any suitable method for measuring mRNA expression in a sample from an individual may be used. For example, the level of PD-L1 mRNA expression may be measured using in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”).

In some embodiments, the percent of cells in the tumor sample that are positive for PD-L1 is measured based on the level of PD-L1 protein in the sample. Any suitable method for measuring protein expression in a sample from an individual may be used. For example, the level of PD-L1 protein expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), proteomics (e.g., mass spectrometry), quantitative blood based assays (as for example serum ELISA), biochemical enzymatic activity assays, or multiplexed immunoassays such as those available from Rules Based Medicine or Meso Scale Discovery (“MSD”). In some embodiments, the percent of cells that are positive for PD-L1 is determined by IHC.

In some embodiments, the percent of cells that are positive for PD-L1 in the tumor sample (e.g. a PD-L1 score) is used to determine in part a composite biomarker score. In some embodiments, the percent of the cells that are positive for PD-L1 is used to determine a composite biomarker score but does not increment the biomarker score. In some embodiments, the percent of cells that are positive for PD-L1 is: (i) less than 1%, (ii) at least 1%, and/or (iii) at least 50%. In some embodiments, the percent of cells that are positive for PD-L1 is: (i) less than 1%, (ii) at least 1%, and/or (iii) at least 50%, and the composite biomarker score is incremented if (ii) is met, and further incremented if (iii) is also met. In some embodiments, the percent of cells that are positive for PD-L1 is: (i) less than 1%, (ii) at least 1%, and/or (iii) at least 50%, and the composite biomarker score is incremented by 1 if (ii) is met and incremented by 2 if both (ii) and (iii) are met.

In some embodiments, the percent of cells that are positive for PD-L1 in the tumor sample is at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of cells in the tumor sample express PD-L1 protein and/or PD-L1 mRNA (e.g., are positive for PD-L1 protein and/or PD-L1 mRNA). In some embodiments, the percent of cells that are positive for PD-L1 in the tumor sample is at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of cells in the tumor sample express PD-L1 protein (e.g., are positive for PD-L1 protein). In some embodiments, the composite biomarker score is incremented. In some embodiments, the composite biomarker score is incremented by one. In some embodiments, the composite biomarker score is incremented by two. In some embodiments, the percent of cells positive for PD-L1 is at least 1% but not more than 49% and the composite biomarker score is incremented by one. In some embodiments, the percent of cells positive for PD-L1 is at least 50% but not more than 100% and the composite biomarker score is incremented by two.

In some embodiments, the percent of cells in the tumor sample that are positive for PD-L1 is determined to be 49% or less positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 46%, at least about 47%, at least about 48%, at least about 49%) of cells in the tumor sample express PD-L1 protein and/or PD-L1 mRNA (e.g., are positive for PD-L1 protein and/or PD-L1 mRNA). In some embodiments, the percent of cells in the tumor sample that are positive for PD-L1 is determined to be 49% or less positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 46%, at least about 47%, at least about 48%, at least about 49%) of cells in the tumor sample express PD-L1 protein (e.g., are positive for PD-L1 protein). In some embodiments, the composite biomarker score is incremented by one. In some embodiments, the composite biomarker score is incremented by two. In some embodiments, the percent of cells positive for PD-L1 is at least 1% but not more than 49% and the composite biomarker score is incremented. In some embodiments, the percent of cells positive for PD-L1 is at least 1% but not more than 49% and the composite biomarker score is incremented by one.

In some embodiments, the percent of cells that are positive for PD-L1 in the tumor sample is at least about 50%, at least about 55%, at least about 60%, at least about 65%, at least about 70%, at least about 75%, at least about 80%, at least about 85%, at least about 90%, at least about 95%, at least about 99%, or 100% of tumor cells express PD-L1 protein and/or PD-L1 mRNA (e.g., are positive for PD-L1 protein and/or PD-L1 mRNA). In some embodiments, the percent of cells that are positive for PD-L1 in the tumor sample is at least about 50%, at least about 55%, at least about 60%, at least about 65%, at least about 70%, at least about 75%, at least about 80%, at least about 85%, at least about 90%, at least about 95%, at least about 99%, or 100%) of tumor cells express PD-L1 protein (e.g., are positive for PD-L1 protein). In some embodiments, the composite biomarker score is incremented. In some embodiments, the composite biomarker score is incremented by two. In some embodiments, the percent of cells positive for PD-L1 is at least 50% but not more than 100% and the composite biomarker score is incremented by two.

In some embodiments, the percent of cells that are positive for PD-L1 in the tumor sample is at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 45%, at least about 50%, at least about 55%, at least about 60%, at least about 65%, at least about 70%, at least about 75%, at least about 80%, at least about 85%, at least about 90%, at least about 95%, at least about 100%) of cells in the tumor sample express PD-L1 protein and/or PD-L1 mRNA (e.g., are positive for PD-L1 protein and/or PD-L1 mRNA). In some embodiments, the percent of cells that are positive for PD-L1 in the tumor sample is at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 45%, at least about 50%, at least about 55%, at least about 60%, at least about 65%, at least about 70%, at least about 75%, at least about 80%, at least about 85%, at least about 90%, at least about 95%, at least about 100%) of cells in the tumor sample express PD-L1 protein (e.g., are positive for PD-L1 protein). In some embodiments, the percent of cells that are positive for PD-L1 is: at least 1%, and/or at least 50%. In some embodiments, the percent of cells that are positive for PD-L1 is at least 1%, and/or at least 50%, and the composite biomarker score is increased if the percent of cells positive for PD-L1 is at least 1%, and further increased if the percent cells that are positive for PD-L1 is at least 50%. In some embodiments, the percent of cells that are positive for PD-L1 is at least 1%, and/or at least 50%, and the composite biomarker score is increased if the percent of cells positive for PD-L1 is at least 1%, and further increased if the percent cells that are positive for PD-L1 is at least 50%. In some embodiments, the percent of cells that are positive for PD-L1 is at least 1% and/or at least 50%, and the composite biomarker score is incremented by 1 if the percent of cells that are positive for PD-L1 is at least 1% is met and incremented by 2 if the percent of cells that are positive for PD-L1 is at least 1% and at least 50%.

In some embodiments, the percent of cells that are positive for PD-L1 in the tumor sample is less than 1% of tumor cells express PD-L1 protein and/or PD-L1 mRNA (e.g., are positive for PD-L1 protein and/or PD-L1 mRNA). In some embodiments, the percent of cells that are positive for PD-L1 in the tumor sample is less than 1% of tumor cells express PD-L1 protein (e.g., are positive for PD-L1 protein. In some embodiments, the percent of cells that are positive for PD-L1 is less than 1% and the composite biomarker score is not incremented. In some embodiments, the percent of cells that are positive for PD-L1 is less than 1% and the composite biomarker score is incremented by 0.

In some embodiments, a plurality of threshold values are defined, wherein the composite biomarker score is incremented based on each threshold met. For illustration, in an exemplary embodiment, two thresholds are defined for the percent of cells in a tumor sample positive for PD-L1: at least 1% and less than 50%, and at least 50%. In this exemplary embodiment, if the assessed percentage of cells in the tumor sample positive for PD-L1 is less than 1%, then the composite biomarker score is not incremented; if the percentage is at least 1% and less than 50%, then the composite biomarker score is incremented (for example, by 1); if the percentage is at least 50%, then the composite biomarker score is incremented by a greater amount (for example, by 2). In this example, an increasing percentage of cells positive for PD-L1 in the tumor sample represents an increased likelihood that the subject is a candidate for treatment with an immune checkpoint inhibitor, and therefore a higher percentage of cells positive for PD-L1 may be weighted to increment the composite biomarker score by a greater amount than a lower percentage of cells positive for PD-L1.

### 3. Gene Mutations

Genetic biomarkers are not limited to germline or somatic gene variants (polymorphisms, mutations), but may also comprise functional deficiencies with a genetic etiology.

Exemplary mutations include, but are not limited to, nucleic acid mutations including single-base substitutions, multi-base substitutions, insertion mutations, deletion mutations, frameshift mutations, missense mutations, nonsense mutations, splice-site mutations, epigenetic modifications (e.g., methylation, phosphorylation, acetylation, ubiquitylation, sumoylation, histone acetylation, histone deacetylation, and the like), and combinations thereof. In some embodiments, the mutation is a “nonsynonymous mutation,” meaning that the mutation alters the amino acid sequence of any of the genes disclosed herein. Such mutations reduce or eliminate protein amounts and/or function by eliminating proper coding sequences required for proper protein translation and/or coding for proteins that are non-functional or have reduced function (e.g., deletion of enzymatic and/or structural domains, reduction in protein stability, alteration of sub-cellular localization, and the like). Both biallelic and monoallelic mutations are contemplated herein. In some embodiments, the inactivating mutation is a loss-of-function mutation in the gene. In some embodiments, the inactivating mutation of is a nonsense, frameshift, or splice-site mutation. Such mutations may lead to lack of expression in cells harboring such mutations.

The following non-limiting methods can be used to identify the presence of an inactivating mutation of in any of the genes disclosed herein, including structural alterations in a nucleic acid and/or polypeptide, changes in copy number of the gene, and changes in expression levels of the gene.

Detection of the mutation may involve the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241: 1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682).

In some embodiments, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA.

In some embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759).

In some embodiments, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 7 4: 5463, and next generation sequencing (NGS).

Other methods for detecting mutations in a biomarker gene include methods in which protection from cleavage agents is used to detect mismatched bases in RNA/RNA or RNA/DNA heteroduplexes (Myers et al. (1985) Science 230: 1242). In some embodiments, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells.

In some embodiments, alterations in electrophoretic mobility can be used to identify mutations in biomarker genes. For example, single strand conformation polymorphism (SSCP) may be used to detect differences in electrophoretic mobility between mutant and wild-type nucleic acids (Orita et al. (1989) Proc Natl. Acad Sci USA 86:2766; see also Cotton (1993) Mutat. Res. 285:125-144 and Hayashi (1992) Genet. Anal. Tech. Appl. 9:73-79).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324: 163; Saiki et al. (1989) Proc. Natl. Acad Sci. USA 86:6230). Such allele specific oligonucleotides are hybridized to PCR amplified target DNA or a number of different mutations when the oligonucleotides are attached to the hybridizing membrane and hybridized with labeled target DNA.

Methods of evaluating the copy number of a biomarker nucleic acid are well known to those of skill in the art. The presence or absence of chromosomal gain or loss can be evaluated simply by a determination of copy number of the regions or markers identified herein.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In some embodiments, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well known in the art to detect RNA can be used, such that higher or lower expression relative to an appropriate control (e.g., a non-amplified portion of the same or related cell tissue, organ, etc.) provides an estimate of the relative copy number of the target nucleic acid. An alternative means for determining genomic copy number is in situ hybridization (e.g., Angerer (1987) Meth. Enzymol 152: 649).

In some embodiments, amplification-based assays can be used to measure copy number. In such amplification-based assays, the nucleic acid sequences act as a template in an amplification reaction (e.g., Polymerase Chain Reaction (PCR)). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present application. In fluorogenic quantitative PCR, quantitation is based on amount of fluorescence signals, e.g., TaqMan and SYBR green.

Gene expression may be assessed by any of a wide variety of well-known methods for detecting expression of a transcribed molecule or protein. Non-limiting examples of such methods include immunological methods, protein purification methods, protein function or activity assays, nucleic acid hybridization methods, nucleic acid reverse transcription methods, and nucleic acid amplification methods.

In some embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. Many techniques are known in the state of the art for determining absolute and relative levels of gene expression, commonly used techniques suitable for use in the present application include Northern analysis, RNase protection assays (RPA), microarrays and PCR-based techniques, such as quantitative PCR and differential display PCR.

In situ hybridization visualization may also be employed, wherein a radioactively labeled anti sense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts.

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In some embodiments, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In some embodiments, the primers and probes hybridize specifically under stringent conditions to a DNA fragment having the nucleotide sequence corresponding to the marker.

The form of labeling of the probes may be any that is appropriate, such as the use of radioisotopes, for example, 32P and 35S. Labeling with radioisotopes may be achieved, whether the probe is synthesized chemically or biologically, by the use of suitably-labeled bases. In some embodiments, the biological sample contains polypeptide molecules from the test subject. Alternatively, the biological sample can contain mRNA molecules from the test subject or genomic DNA molecules from the test subject.

In some embodiments, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

The activity or level of a biomarker protein can be detected and/or quantified by detecting or quantifying the expressed polypeptide. The polypeptide can be detected and quantified by any of a number of means well known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to an immune checkpoint therapy. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder¬ligand assays, immunohistochemical techniques, agglutination, complement assays, high performance liquid chromatography (HPLC), thin layer chromatography (TLC), hyperdiffusion chromatography, and the like.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a Kd of at most about 10⁻⁶M, 10⁻⁷M, 10⁻⁸M, 10⁻⁹M, or less. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

Epigenetic modifications may be detected using known methods in the art. For example, bisulfite conversion of methylated DNA followed by sequencing (e.g., NGS or Sanger sequencing), microarray analysis, qPCR, or PCR; or DNA enzyme digestion of methylated DNA followed by qPCR, PCR, sequencing (e.g., NGS or Sanger sequencing), HPLC-UV, LC-MS/MS or ELISA assay. In a bisulfite conversion method, a DNA sample is treated with sodium bisulfite resulting in the deamination of unmethylated cytosine to uracil and allowing the distinction between cytosine and methylated cytosine. The DNA enzyme digestion method is based on the use of DNA endonucleases which do not cut methylated DNA. Digestion of specific DNA target sequences by these enzymes generates DNA fragments of different lengths which may be sequenced to determine the extent of methylation.

Individual biomarkers may be used in the methods disclosed herein to determine a composite biomarker score.

a. ARID1A

In some embodiments, the methods provided herein comprise determining a composite marker score, wherein the composite biomarker score is determined based, at least in part, on the presence of a cancer-associated mutation in a ARID1A gene in the sample. The methods described herein also relate to detection of inactivating mutations of ARID1A. An exemplary nucleic acid sequence of human ARID1A is available as GenBank accession NM_006015. In some embodiments, when a cancer-associated mutation, such as an inactivating mutation, is present in an ARID1A gene in the sample, the composite biomarker score is incremented.

The AT-rich interactive domain-containing protein 1A (ARID1A) gene encodes the basic directional subunit of SWI/SNF chromatin remodeling complexes. In humans, it contains 20 exons and encodes two functionally identical 2285 and 2086 amino acid isoforms of the ARID1A protein, also known as Brahma-related associated factor 250a (BAF 250a), SWI/SNF-related matrix-associated actin-dependent regulators of chromatin factor 1 (SMARCF1), or p270. Through its interactions with nucleosomal DNA, transcription factors and nuclear hormone receptors, it plays a key role in regulating cellular proliferation, gene expression and the repair of genetic material, while the loss of its expression triggers carcinogenesis. A total of 97% of inactivating ARID1A somatic mutations that lead to the reduction or complete loss of protein expression are nonsense, point and insertion or deletion frameshift mutations, distinctive of tumor suppressor genes, that have been found to be distributed throughout its length. The consequential abnormal mRNA often carries premature stop codons and is translated into a truncated protein, functionally degraded, either due to misfolding or as it is partially incomplete, resulting in the disturbance of the normal levels of nuclear ARID1A and the destabilization of SWI/SNF complexes. Mutations of tumor suppressor genes usually include alterations in both alleles. However, in the case of ARID1A, one allele mutation is sufficient to cause the loss of ARID1A expression in the majority of heterozygous tumors, thus indicating genetic haplodeficiency. In human cancer, ARID1A regulates cell cycle progression and prevents genomic instability. Known ARID1A mutations associated with cancer are available at databases known to one skilled in the art such as the Vanderbilt-Ingram Cancer Center My Cancer Genome database, available at www.mycancergenome.org/content/gene/ARID1A/.

In some embodiments, the methods comprise determining a composite biomarker score and determining, based on the determined composite biomarker score, whether the subject will likely benefit from the treatment comprising the immune checkpoint inhibitor. In some embodiments, the methods comprise determining the composite biomarker score, wherein the composite biomarker score is determined based, at least in part, on the presence of a cancer-associated mutation in the ARID1A gene in the sample. In some embodiments, the composite biomarker may be determined without determining the presence of a cancer-associated mutation in a ARID1A gene in the sample. In some embodiments, the composite biomarker score is incremented if the sample comprises the presence of a cancer-associated mutation in the ARID1A gene in the sample. In an exemplary embodiment, the composite biomarker score is incremented by 1 if the sample comprises a cancer-associated mutation in the ARID1A gene in the sample. In an exemplary embodiment, the composite biomarker score is not incremented if the sample does not comprise a cancer-associated mutation in the ARID1A gene in the tumor sample.

In some embodiments, the methods further comprise providing a report to a party. In some embodiments, the alteration comprises one or more of a substitution of one or more nucleotides, an insertion of one or more nucleotides, or a deletion of one or more nucleotides in ARID1A. In some embodiments, the alteration in ARID1A is an inactivating mutation, a loss-of-function mutation, a copy number alteration (e.g., a deletion), a rearrangement, or a gene fusion. In some embodiments, the alteration in ARID1A results in an inactive polypeptide or protein encoded by ARID1A. In some embodiments, the alteration in ARID1A results in a polypeptide or protein encoded by ARID1A that has reduced activity, e.g., as compared to a polypeptide or protein encoded by an ARID1A gene without the alteration. In some embodiments, the alteration in ARID1A results in reduced expression of ARID1A. In some embodiments, the alteration in ARID1A results in loss of expression of ARID1A.

b. KEAP1/NFE2L2 Pathway

In some embodiments, the composite biomarker score is based, at least in part, on the presence of a cancer-associated mutation in a KEAP1/NFE2L2 pathway gene. These cancer-associated mutations, in some embodiments, act as a “negative” biomarker, wherein the absence of a mutation increments the composite biomarker score and the presence of a mutation does not increment the composite biomarker score. Accordingly, in some embodiments, the methods provided herein comprise determining a composite marker score, wherein the composite biomarker score is determined based on, at least in part, the presence of one or more cancer associated mutations in the KEAP1/NFE2L2 pathway in the sample. In some embodiments, the cancer-associated mutation(s) in the KEAP1/NFE2L2 pathway relate to inactivating mutations of KEAP1/NFE2L2, which reduce the expression and/or activity of KEAP1/NFE2L2 pathway. Thus, in some embodiments, when the tumor sample is found to have reduced expression and/or activity of the KEAP1/NFE2L2 pathway, the composite biomarker score is not incremented. In some embodiments, when the tumor sample is found to not have reduced expression and/or activity of the KEAP1/NFE2L2 pathway, such as when compared to normal tissue or other control sample, then the composite biomarker score is incremented. In some embodiments, negative biomarkers are combined with positive biomarkers to determine the composite biomarker score.

An exemplary nucleic acid sequence of a human KEAP1 gene is available at GenBank accession NM_012289. An exemplary nucleic acid sequence of a human NFE2L2 gene is available at GenBank accession NM_006164.

The KEAP1/NFE2L2 pathway is the principal protective response to oxidative and electrophic stresses. Under homeostatic conditions, KEAP1 forms part of an E3 ubiquitin ligase, which tightly regulates the activity of the transcription factor NFE2L2 by acting as a substrate adaptor and targeting it for ubiquitination and proteasome-dependent degradation. Therefore, KEAP1 is a suppressor of nuclear factor (erythroid-derived 2)-like 2 (NFE2L2/NFE2L2)).

The KEAP1/NFE2L2 axis plays a major role in the cellular regulation of redox homeostasis, mitochondrial physiology, autophagy, proteostasis, immune system, and metabolism. The NFE2L2/KEAP1 complex is mediated by activating stimulation, interaction with other transcription factors, activators or repressors, and crosstalk with other signaling pathways. At the center of a complex regulatory network, the KEAP1/NFE2L2 pathway is emerging as a critical regulator of metabolism in cancer cells as its interactions with the metabolism-related pathway including the PI3K/AKT/mTOR pathway, p62 pathway, AMPK, and TCA cycle have been revealed. In addition, the NFE22L2/KEAP1 axis contributes to several metabolic processes in cancers and the production of metabolites that promote cell proliferation and survival. In particular, the constitutive overexpression of NFE2L2′ accelerates the proliferation of cancer cells, which is the result of the reprogramming of intracellular anabolic and catabolic metabolism.

Mutations in the KEAP1/NFE2L2 pathway are known to be involved in malignant transformation of various cancer types. Loss-of function mutations lead to an increase of NFE2L2 in the nucleus. Gain of function mutations of NFE2L2 are found near or within pivotal binding sites and interrupt binding of NFE2L2 to KEAP1 dimers. This leads to an increase in intracellular NRF2, (ii) the synthesis of antioxidant and detoxification enzymes, and (iii) the production of drug efflux pumps in cancer cells. KEAP1 or NFE2L2 mutations promote cell proliferation in tumors and may also participate in causing resistance to chemotherapy. Downregulation of NFE2L2 or overexpression of KEAP1 both triggered chemotherapy sensitivity.

Known KEAP1 mutations associated with cancer are available at databases known to one skilled in the art such as at the Vanderbilt-Ingram Cancer Center My Cancer Genome database at www.mycancergenome.org/content/gene/KEAP1/. Similarly, NFE2L2 gain of function mutations or NFE2L2 mutations associated with cancer are available at databases known to one skilled in the art such as the Vanderbilt-Ingram My Cancer Genome database at www.mycancergenome.org/content/gene/NFE2L2/.

In some embodiments, the methods comprise determining a composite biomarker score and determining, based on the determined composite biomarker score, whether the subject will likely benefit from the treatment comprising the immune checkpoint inhibitor. In some embodiments, the methods comprise determining the composite biomarker score, wherein the composite biomarker score is determined based on, at least in part, the presence or absence of a cancer-associated mutation in a KEAP1 and/or NFE2L2 gene in the sample. In some embodiments, the composite biomarker may be determined without determining the presence of a cancer-associated mutation in a KEAP1 and/or NFE2L2 gene in the sample. In some embodiments, the composite biomarker may be determined with determining the presence of a cancer-associated mutation in the KEAP1 and/or NFE2L2 gene in the sample. In some embodiments, the composite biomarker score is not incremented if the sample comprises the presence of a cancer-associated mutation in the KEAP1 and/or NFE2L2 gene in the sample. In some embodiments, the composite biomarker score is not incremented if the sample comprises the presence of a cancer-associated mutation in the KEAP1 and/or NFE2L2 gene in the sample. In some embodiments, the composite biomarker score is incremented if the sample does not comprise the presence of a cancer-associated mutation in the KEAP1 and/or NFE2L2 gene in the sample. In some embodiments, the composite biomarker score is incremented by 1 if the sample does not comprise the presence of a cancer-associated mutation in the KEAP1 and/or NFE2L2 gene in the sample. In some embodiments, the composite biomarker score is incremented if the sample comprises the presence of a cancer-associated mutation in the KEAP1 and/or NFE2L2 gene in the sample. In some embodiments, the composite biomarker score is not incremented if the sample does not comprise the presence of a cancer-associated mutation in the KEAP1 and/or NFE2L2 gene in the sample. In an exemplary embodiment, the composite biomarker score is incremented by 0 if the sample does not comprise the presence of a cancer-associated mutation in the KEAP1 and/or NFE2L2 gene in the sample. In another exemplary embodiment, the composite biomarker score is decreases by 1 if the sample comprises the presence of a cancer-associated mutation in the KEAP1 and/or NFE2L2 gene in the sample.

In some embodiments, the alteration comprises one or more of a substitution of one or more nucleotides, an insertion of one or more nucleotides, or a deletion of one or more nucleotides in KEAP1 and/or NFE2L2. In some embodiments, the alteration in KEAP1 is an inactivating mutation, a loss-of-function mutation, a copy number alteration (e.g., a deletion), a rearrangement, or a gene fusion. In some embodiments, the alteration in KEAP1 results in an inactive polypeptide or protein encoded by KEAP1. In some embodiments, the alteration in KEAP1 results in a polypeptide or protein encoded by KEAP1 that has reduced activity, e.g., as compared to a polypeptide or protein encoded by an KEAP1 gene without the alteration. In some embodiments, the alteration in KEAP1 results in reduced expression of KEAP1. In some embodiments, the alteration in KEAP1 results in loss of expression of KEAP1. In some embodiments, the alteration comprises one or more of a substitution of one or more nucleotides, an insertion of one or more nucleotides, or a deletion of one or more nucleotides in NFE2L2. In some embodiments, the alteration in NFE2L2 is an activating mutation, a gain-of-function mutation, a copy number alteration (e.g., a deletion or duplication), a rearrangement, or a gene fusion. In some embodiments, the alteration in NFE2L2 results in a gain-of-function polypeptide or protein encoded by NFE2L2. In some embodiments, the alteration in NFE2L2 results in a polypeptide or protein encoded by NFE2L2 that has increased activity, e.g., as compared to a polypeptide or protein encoded by an NFE2L2 gene without the alteration. In some embodiments, the alteration in NFE2L2 results in increased expression of NFE2L2.

c. STK11/LKB1

In some embodiments, the composite biomarker score is based, at least in part, on the presence of a cancer-associated mutation in a STK11/LKB1 gene. These cancer-associated mutations, in some embodiments, act as a “negative” biomarker, wherein the absence of a mutation increments the composite biomarker score and the presence of a mutation, such as a mutation leading to loss of STK11/LKB1 function (such as loss of kinase activity) or gene expression, does not increment the composite biomarker score. Thus, in some embodiments, when the tumor sample is found to have a mutation in a STK11/LKB1 gene, the composite biomarker score is not incremented. In some embodiments, when the tumor sample is found to have a mutation in a STK11/LKB1 gene, the composite biomarker score is incremented. In some embodiments, negative biomarkers are combined with positive biomarkers to determine the composite biomarker score.

In some embodiments, the methods provided herein comprise determining a composite biomarker score, wherein the composite biomarker score is determined based on, at least in part, the presence of one or more cancer-associated mutations in a serine threonine kinase 11 (STK11) gene (also known as liver kinase B1; LKB1) in a tumor sample. In some embodiments, the cancer-associated mutation pertains to inactivating mutations of STK11/LKB1, which reduce the expression and/or activity of STK1/LK 31.

An exemplary nucleic acid sequence of a STK11/LKB1 gene is available at GenBank accession NM_000455.

STK11/LKB1 is located at the telomeric region of the short arm of chromosome 19 (19p13.3). Nine exons code for a sequence of 443-amino acids which form STK11/LKB1. It is an essential master upstream kinase that activates AMP-activated protein kinase (AMPK) in case of energy deprivation. AMPK is a central metabolic checkpoint in the cell that regulates glucose and lipid metabolism in response to nutrients and energy variations, as well as other cellular functions such as autophagy and polarity. Thus, under nutrient deprivation or hypoxia, an AMP accumulation occurs, in conjunction with ATP depletion, leading to the direct activation of AMPK by STK11/LKB1. Activation of AMPK in conjunction with other regulators allows a switch from an anabolic to a catabolic metabolism, promoting cell survival in energy stress conditions. It triggers physiological processes that regenerate ATP, as well as activating 12 other kinases of the AMPK subfamily. The STK11/LKB1/AMPK axis is also involved in the glucose catabolism by an increase in glucose uptake and the promotion of the glycolysis process. STK11/LKB1 also promotes apoptosis.

STK11/LKB1 loss of function has been found in several cancer types, mainly through somatic alterations in the STK11 gene such as non-sense mutation, loss of heterozygosity, insertions, intragenic deletions, or chromosomal deletions. While most mutations affect the kinase domain resulting in loss of kinase activity, others affect production, stability, or localization of the protein. Meanwhile, other non-mutational mechanisms result in an alteration of STK11/LKB1 expression. STK11/LKB1 expression can be negatively regulated by hyper-methylation of the STK11 promoter region. Protein translation can also be reduced at the post-translational level by microRNAs, as shown in cervical and head and neck cancer. These non-mutational mechanisms should be considered in tumor characterization because such tumors could have growth and aggressiveness similar to STK11-mutant tumors. Thus, while most clinical studies have used sequencing to characterize STK11 status, mutation analysis of the STK11 gene may not be sufficient to identify patients with impaired oncosuppressive STK11/LKB1 activity.

Known STK11/LKB1 mutations associated with cancer are available at databases known to one skilled in the art such as at the Vanderbilt-Ingram Cancer Center My Cancer Genome database, available at www.mycancergenome.org/content/gene/STK11/.

In some embodiments, the methods comprise determining a composite biomarker score and determining, based on the determined composite biomarker score, whether the subject will likely benefit from the treatment comprising the immune checkpoint inhibitor. In some embodiments, the methods comprise determining the composite biomarker score, wherein the composite biomarker score is determined based on, at least in part, the presence of a cancer-associated mutation in the STK11/LKB1 gene in the sample. In some embodiments, the composite biomarker may be determined without determining the presence of a cancer-associated mutation in a STK11/LKB1 gene in the sample. In some embodiments, the composite biomarker is determined based on, at least in part, the presence of a cancer-associated mutation in the STK11/LKB1 gene in the tumor sample. In some embodiments, the composite biomarker score is incremented if the sample comprises a cancer-associated mutation in a STK11/LKB1 gene. In an exemplary embodiment, the composite biomarker score is incremented by 1 if the sample comprises the presence of a cancer-associated mutation in the STK11/LKB1 gene in the sample. In some embodiments, the composite biomarker score is incremented by 0 if the sample does not comprise the presence of a cancer-associated mutation in the STK11/LKB1 gene in the sample. In some embodiments, the composite biomarker score is not incremented if the sample does not comprise the presence of a cancer-associated mutation in the STK11/LKB1 gene in the sample. In some embodiments, the composite biomarker score is incremented if the sample comprises the presence of a cancer-associated mutation in the STK11/LKB1 gene in the sample. In some embodiments, the composite biomarker score is decreased if the sample comprises the presence of a cancer-associated mutation in the STK11/LKB1 gene in the sample. In some embodiments, the composite biomarker score is not incremented if the sample does not comprise the presence of a cancer-associated mutation in the STK11/LKB1 gene in the sample. In an exemplary embodiment, the composite biomarker score is incremented by 0 if the sample does not comprise the presence of a cancer-associated mutation in the STK11/LKB1 gene in the sample. In another exemplary embodiment, the composite biomarker score is decreased by 1 if the sample comprises the presence of a cancer-associated mutation in the STK11/LKB1 gene in the sample.

In some embodiments, the alteration comprises one or more of a substitution of one or more nucleotides, an insertion of one or more nucleotides, or a deletion of one or more nucleotides in STK11/LKB1. In some embodiments, the alteration in STK11/LKB1 is an inactivating mutation, a loss-of-function mutation, a copy number alteration (e.g., a deletion), a rearrangement, or a gene fusion. In some embodiments, the alteration in STK11/LKB1 results in an inactive polypeptide or protein encoded by STK11/LKB1. In some embodiments, the alteration in STK11/LKB1 results in a polypeptide or protein encoded by STK11/LKB1 that has reduced activity, e.g., as compared to a polypeptide or protein encoded by an STK11/LKB1 gene without the alteration. In some embodiments, the alteration in STK11/LKB1 results in reduced expression of STK11/LKB1. In some embodiments, the alteration in STK11/LKB1 results in loss of expression of STK11/LKB1.

d. CDKN2A

In some embodiments, the composite biomarker score is determined based on, at least in part, the functional loss of a CDKN2A gene expression. This functional loss may be the result of, for example, mutations or cancer-associated mutations in the CDKN2A gene. In some embodiments, the mutation or cancer-associated mutations in the CDKN2A gene is a homozygous deletion of CDKN2A. In some embodiments, the methods provided herein comprise determining a composite marker score, wherein the composite biomarker score is determined based on, at least in part, the presence of a cancer associated mutation in the CDKN2A gene in the sample. Functional loss of CDKN2A gene expression may, in some embodiments, act as a “negative” biomarker, wherein normal expression increments the composite biomarker score and loss of function does not increment the composite biomarker score. Functional loss of CDKN2A gene expression may, in some embodiments, act as a “negative” biomarker (e.g., increment by a negative value), wherein any normal expression does not increment the composite biomarker score and total loss of function (e.g. a homozygous deletion of CDKN2A) decreases the composite biomarker score (e.g., decreases the composite biomarker score by 1). Thus, in some embodiments, when the tumor sample is found to have functional loss of CDKN2A gene expression, such as compared to normal tissue or other suitable control, the composite biomarker score is not incremented. In some embodiments, when the tumor sample is found to not have functional loss of CDKN2A gene expression, such as compared to normal tissue or other suitable control, the composite biomarker score is incremented. In some embodiments, when the tumor sample is found to have functional loss of CDKN2A gene expression, such as compared to normal tissue or other suitable control, the composite biomarker score is decreased. In some embodiments, when the tumor sample is found to not have functional loss of CDKN2A gene expression, such as compared to normal tissue or other suitable control, the composite biomarker score is not incremented.

An exemplary nucleic acid sequence of a CDKN2A gene is available at GenBank accession NM_000077.

The Cyclin-dependent kinase inhibitor 2A (CDKN2A) gene encodes several protein isoforms that function as inhibitors of CDK4 and ARF. The most well-studied are the p16(INK4A) and the p14(ARF) proteins. Both function as tumor suppressors, which means they keep cells from growing and dividing too rapidly or in an uncontrolled manner. Both proteins are also involved in stopping cell division in older cells (i.e., cellular senescence). The p16(INK4A) protein attaches (binds) to two other proteins called CDK4 and CDK6. These proteins help regulate the cell cycle, which is the cell's way of replicating itself in an organized, step-by-step fashion. CDK4 and CDK6 normally stimulate the cell to continue through the cycle and divide. However, binding of p16(INK4A) blocks CDK4's or CDK6's ability to stimulate cell cycle progression. In this way, p16(INK4A) controls cell division. Cells begin to produce p16 (INK4A) when they are no longer able to undergo cell division. The p14(ARF) protein protects a different protein called p53 from being broken down. The p53 protein is an important tumor suppressor that is essential for regulating cell division, senescence, and self-destruction (apoptosis). By protecting p53, p14(ARF) also helps prevent tumor formation. The p14(ARF) and p53 proteins are often made in cells that are unable to undergo cell division.

CDKN2A missense mutations, nonsense mutations, silent mutations, in-frame deletions, frameshift deletions and insertions, and whole gene deletions are observed in cancer such as cancers of the genital tract, mesothelioma, ovarian cancer, skin cancer, and multiple other cancer types. CDKN2A gene is associated with poor prognosis in a variety of cancers. Known CDKN2A mutations associated with cancer are available at databases known to one skilled in the art such as at the Vanderbilt-Ingram Cancer Center My Cancer Genome database, available at www.mycancergenome.org/content/gene/CDKN2A/.

In some embodiments, the methods comprise determining a composite biomarker score and determining, based on the determined composite biomarker score, whether the subject will likely benefit from the treatment comprising the immune checkpoint inhibitor. In some embodiments, the methods comprise determining the composite biomarker score, wherein the composite biomarker score is determined based, at least in part, the presence of a cancer-associated mutation in a CDKN2A gene in the sample. In some embodiments, the composite biomarker may be determined without determining the presence of a cancer-associated mutation in a CDKN2A gene in the sample. In some embodiments, the composite biomarker score may be determined based on, at least in part, determining the presence of a cancer-associated mutation in the CDKN2A gene in the sample. In some embodiments, the composite biomarker score may be determined based on, at least in part, determining the presence of a cancer-associated mutation in the CDKN2A gene in the sample, wherein the cancer associated mutation in the CDKN2A is a homozygous deletion. In some embodiments, the composite biomarker score is not incremented if the sample comprises the presence of a cancer-associated mutation in the CDKN2A gene in the sample. In some embodiments, the composite biomarker score is not incremented if the sample comprises the presence of a cancer-associated mutation in the CDKN2A gene in the sample. In an exemplary embodiment, the composite biomarker score is incremented by 1 if the sample does not comprise the presence of a cancer-associated mutation in the CDKN2A gene in the sample. In some embodiments, the composite biomarker score is incremented if the sample comprises the presence of a cancer-associated mutation in the CDKN2A gene in the sample. In some embodiments, the composite biomarker score is not incremented if the sample does not comprise the presence of a cancer-associated mutation in the CDKN2A gene in the sample. In an exemplary embodiment, the composite biomarker score is incremented by 0 if the sample does not comprise the presence of a cancer-associated mutation in the CDKN2A gene in the sample. In another exemplary embodiment, the composite biomarker score is decreased by 1 if the sample comprises the presence of a cancer-associated mutation in the CDKN2A gene in the sample. In some embodiments, the cancer-associated mutation is a homozygous deletion in the CDKN2A gene.

In some embodiments, the alteration comprises one or more of a substitution of one or more nucleotides, an insertion of one or more nucleotides, or a deletion of one or more nucleotides in CDKN2A. In some embodiments, the alteration in CDKN2A is an inactivating mutation, a loss-of-function mutation, a copy number alteration (e.g., a deletion), a rearrangement, or a gene fusion. In some embodiments, the alteration in CDKN2A results in an inactive polypeptide or protein encoded by CDKN2A. In some embodiments, the alteration in CDKN2A results in a polypeptide or protein encoded by CDKN2A that has reduced activity, e.g., as compared to a polypeptide or protein encoded by an CDKN2A gene without the alteration. In some embodiments, the alteration in CDKN2A results in reduced expression of CDKN2A. In some embodiments, the alteration in CDKN2A results in loss of expression of CDKN2A.

e. DNA Damage Response (DDR)

In some embodiments, the methods provided herein comprise determining a composite marker score, wherein the composite biomarker score is determined based on a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and/or BARD1) in the sample. The inability to repair DNA damage properly in mammals leads to various disorders and enhanced rates of tumor development. Organisms respond to chromosomal insults by activating a complex damage response pathway. This pathway regulates known responses such as cell-cycle arrest and apoptosis (programmed cell death) and has recently been shown to control additional processes including direct activation of DNA repair networks. Cancer is an evolutionary disease fueled by genomic instability. The majority of cancers display chromosomal instability (CIN), which is characterized by alterations of chromosomal numbers and/or structure. Other forms of genomic instability include accumulation of DNA base mutations and microsatellite instability (MIN), which results in contraction or expansion of the number of repetitive microsatellite sequences. Maintenance of genomic integrity by the DDR is critical to prevent tumorigenesis, as indicated by the cancer-prone phenotype of several DDR syndromes. In some embodiments, the composite biomarker score is determined based on, at least in part, one or more cancer-associated mutations in at least one DNA damage response (DDR) gene in the sample. In some embodiments, the at least one DDR gene is at least one of ATM, BRCA2, BRIP1, MRE11, POLE, MSH2, PARP1, MLH1, MSH6, PMS2, BRCA1, PALB2, and BARD1. In some embodiments, the at least one DDR gene is a plurality of ATM, BRCA2, BRIP1, MRE11, POLE, MSH2, PARP1, MLH1, MSH6, PMS2, BRCA1, PALB2, and BARD1. In some embodiments, the at least one DDR gene includes each of ATM, BRCA2, BRIP1, MRE11, POLE, MSH2, PARP1, MLH1, MSH6, PMS2, BRCA1, PALB2, and BARD1. In some embodiments, the composite biomarker score is incremented if there is a functional reduction in the DDR response. In some embodiments, the composite biomarker score is not incremented if there is no functional reduction, such as compared to normal tissue or other suitable control, in the DDR response. Such functional reduction may be assessed by detecting the presence of a cancer-associated mutation, or multiple cancer-associated mutations, in one or more of ATM, BRCA2, BRIP1, MRE11, POLE, MSH2, PARP1, MLH1, MSH6, PMS2, BRCA1, PALB2, and/or BARD1.

Ataxia-Telangiectasia Mutated (ATM) is a serine/threonine kinase that is recruited and activated by DNA double-strand breaks. It phosphorylates several key proteins that initiate activation of the DNA damage checkpoint, leading to cell cycle arrest, DNA repair or apoptosis. Several of these targets, including p53, CHK2, BRCA1, NBS1 and H2AX are tumor suppressors. Defects in ATM thus confer a loss of the ability to repair certain types of damage to DNA, and cancer may result from improper repair. An exemplary nucleic acid sequence of an ATM gene is available as Transcript ID NM_000051, available at the website: www.ncbi.nlm.nih.gov/nuccore/NM_000051. ATM loss of function mutations associated with cancer are available at databases known to one skilled in the art such as, www. mycancergenome.org/content/gene/ATM/.

Breast cancer type 1 susceptibility protein (BRCA1) is critical for the repair of double stranded breaks (DSBs) by homologous recombination repair, and loss of function results in increased mutation and genome instability. It is this increased genomic instability that is thought to be responsible for the significantly increased cancer risk of patients with familial or germline BRCA (gBRCA) mutations. An exemplary nucleic acid sequence of a BRCA1 gene is available at NCBI Reference Sequence: NM_007294. BRCA1 loss of function mutations associated with cancer are available at databases known to one skilled in the art, such as the Vanderbilt-Ingram Cancer Center My Cancer Genome database.

Breast cancer type 2 susceptibility protein (BRCA2) is critical for the repair of DSBs by homologous recombination repair, and loss of function results in increased mutation and genome instability. It is this increased genomic instability that is thought to be responsible for the significantly increased cancer risk of patients with familial or germline BRCA mutations. An exemplary nucleic acid sequence of a BRCA2 gene is available as Transcript ID NM_000059, available at the website: www.ncbi.nlm.nih.gov/nuccore/NM_000059. BRCA2 loss of function mutations associated with cancer are available at databases known to one skilled in the art such as, www.mycancergenome.org/content/gene/BRCA2/.

The BRCA1 interacting protein 1 (BRIP1) gene encodes the Fanconi anemia group J protein which has 5′ to 3′ helicase activity important for unwinding D-loops and may be involved in resolving RAD51 nucleofilaments. BRIP1 is a known tumor suppressor gene that has vital function in preserving the genetic stability by repairing DNA damage. An exemplary nucleic acid sequence of a BRIP1 gene is available as Transcript ID NM_032043, available at the website: www.ncbi.nlm.nih.gov/nuccore/NM_032043. BRIP1 loss of function mutations associated with cancer are available at databases known to one skilled in the art such as, www. mycancergenome.org/content/gene/BRIP1/.

Meiotic recombination 11 homolog A (MRE11) is a gene that encodes a nuclear protein that functions in homologous recombination, the maintenance of telomere length, and DNA double-strand break repair. Missense mutations, nonsense mutations, silent mutations, and frameshift deletions are observed in cancers. Exemplary nucleic acid sequences of a MRE11 gene is available as Transcript ID NM_005590, available at the website: www.ncbi.nlm.nih.gov/nuccore/NM_005590; Transcript ID NM_005591, available at the website: www.ncbi.nlm.nih.gov/nuccore/NM_005591; Transcript ID NM_001330347, available at the website: www.ncbi.nlm.nih.gov/nuccore/NM_001330347. MRE11 mutations associated with cancer are available at databases known to one skilled in the art such as, www. mycancergenome.org/content/gene/MRE11A/.

DNA polymerase epsilon, encoded by the POLE gene, is a critical protein involved in DNA proofreading and replication. POLE synthesizes the leading strand of DNA in the replication fork and has a 3′-5′ exonuclease domain that increases replication accuracy by approximately 100-fold through recognition and excision of mismatched base pairs. Somatic and germline POLE proofreading defects, particularly mutations occurring in the exonuclease domain representing codons 268-471, are more often found in mismatch repair proficient tumors and associated with hypermutagenesis. DNA polymerase epsilon is implicated to play a major role in DNA synthesis of the leading strand. In some cancer types, especially colorectal and endometrial cancers, polymerase epsilon is mutated at several hotspots, causing large amounts of mutations, termed ultramutation. An exemplary nucleic acid sequence of a POLE gene is available as Transcript ID NM_006231, available at the website: www.ncbi.nlm.nih.gov/nuccore/NM_006231. POLE mutations associated with cancer are available at databases known to one skilled in the art such as, www. mycancergenome.org/content/gene/POLE/.

MutS homolog 2 (MSH2) gene encodes a protein that functions in DNA-mismatch repair. Mutations in MSH2 and other mismatch repair genes cause DNA damage to go unrepaired, resulting in an increase in mutation frequency. If DNA repair is deficient, DNA damage tends to accumulate. Such excess DNA damage may increase mutations due to error-prone translesion synthesis and error prone repair (see e.g. microhomology-mediated end joining). Elevated DNA damage may also increase epigenetic alterations due to errors during DNA repair. Such mutations and epigenetic alterations may give rise to cancer. An exemplary nucleic acid sequence of a MSH2 gene is available as Transcript ID NM_000251, available at the website: www.ncbi.nlm.nih.gov/nuccore/NM_000251. MSH2 mutations associated with cancer are available at databases known to one skilled in the art such as, www. mycancergenome.org/content/gene/msh2/.

Poly (ADP-ribose) polymerase 1 (PARP1) is a gene that encodes a protein that is involved in the regulation of cellular processes such as differentiation, proliferation, tumor transformation, and recovery from DNA damage. PARP1 is a major factor in the repair of single stranded breaks (SSBs), and its enzymatic activity is required for both relaxing chromatin and PARP dissociation from the DNA that occurs following auto-modification. Both events are required to facilitate SSB repair. An important consequence of this is that trapped PARP-DNA complexes can lead to the stalling and/or collapsing of replication forks, resulting in the generation of more deleterious DSBs. An exemplary nucleic acid sequence of a PARP1 gene is available as Transcript ID NM_001618, available at the website: www.ncbi.nlm.nih.gov/nuccore/NM_001618. PARP1 mutations associated with cancer are available at databases known to one skilled in the art such as, www. mycancergenome.org/content/gene/PARP1/.

The MutL protein homolog 1 (MLH1) protein forms a complex with PMS2 (see below). Together, PMS2-MLH1 is responsible for 3′ nick-directed mismatch repair, repairs DNA mismatches and insertion-deletion errors that form from DNA replication and recombination events. An exemplary nucleic acid sequence of a PMS2 gene is available at NCBI Reference Sequence NM_000535. An exemplary nucleic acid sequence of a MLH1 gene is available at NCBI Reference Sequence NM_000249. MLH1 loss of function mutations associated with cancer are available at databases known to one skilled in the art, such as the Vanderbilt-Ingram Cancer Center My Cancer Genome database.

MutS Homologue 6 (MSH6) is part of the MSH2-MSH6 complex that is responsible mismatch recognition in DNA, and for initiating repair of single base mismatches and short insertion deletion loops during DNA replication. Thus, MSH2-MSH6 is critical for maintaining genome integrity. An exemplary nucleic acid sequence of a MSH6 gene is available at NCBI Reference Sequence NM_000179. MSH6 loss of function mutations associated with cancer are available at databases known to one skilled in the art, such as the Vanderbilt-Ingram Cancer Center My Cancer Genome database.

The postmeiotic segregation increased 2 (PMS2) protein forms a heterodimer with MutL homolog 1 (MLH1). Together, PMS2-MLH1 is responsible for 3′ nick-directed mismatch repair, repairs DNA mismatches and insertion-deletion errors that form from DNA replication and recombination events. An exemplary nucleic acid sequence of a PMS2 gene is available at NCBI Reference Sequence NM_000535. PMS2 loss of function mutations associated with cancer are available at databases known to one skilled in the art, such as the Vanderbilt-Ingram Cancer Center My Cancer Genome database.

Partner and Localizer of BRCA2 (PALB2) binds to BRCA2. PALB2 is involved in homologous recombination repair by binding to single stranded DNA and interacts with RAD51. This promotes strand invasion and homologous recombination. An exemplary nucleic acid sequence of a PALB2 gene is available at NCBI Reference Sequence NM_024675. PALB2 loss of function mutations associated with cancer are available at databases known to one skilled in the art, such as the Vanderbilt-Ingram Cancer Center My Cancer Genome database.

BRCA1-associated RING domain 1 (BARD1) protein is a binding partner of BRCA1. BARD1 complexes with BRCA1 to form a ubiquitin ligase that targets proteins involved in DNA repair and cell cycle regulation. On its own, BARD1 also has a role in regulating genome stability and regulating proliferation. Loss of BARD1 function results in dysregulated cellular proliferation. An exemplary nucleic acid sequence of a BARD1 gene is available at NCBI Reference Sequence NM_000465. BARD1 loss of function mutations associated with cancer are available at databases known to one skilled in the art, such as the Vanderbilt-Ingram Cancer Center My Cancer Genome database.

In some embodiments, the methods comprise determining a composite biomarker score and determining, based on the determined composite biomarker score, whether the subject will likely benefit from the treatment comprising the immune checkpoint inhibitor. In some embodiments, the methods comprise determining the composite biomarker score, wherein the composite biomarker score is determined based, in part, on the presence of a cancer-associated mutation in a DDR gene in the sample. In some embodiments, the DDR gene comprise one or more of ATM, BRCA2, BRIP1, MRE11, POLE, MSH2, PARP1, MLH1, MSH6, PMS2, BRCA1, PALB2, and BARD1. In some embodiments, the methods comprise determining the composite biomarker score, wherein the composite biomarker score is determined based, in part, on the presence of one or more cancer-associated mutation in a DDR gene in the sample. In some embodiments, the composite biomarker may be determined without including determining the presence of a cancer-associated mutation in all of the DDR genes described in this section in the sample. In some embodiments, the composite biomarker may be determined with determining the presence of a cancer-associated mutation in the DDR gene in the sample. In some embodiments, the composite biomarker may be determined with determining the presence of a cancer-associated mutation in at least one DDR gene in the sample. In some embodiments, the cancer-associated mutation reduces gene expression of one or more DDR genes in the sample. In some embodiments, the cancer-associated mutation reduces gene expression of one or more genes selected from the list consisting of ATM, BRCA2, BRIP1, MRE11, POLE, MSH2, PARP1, MLH1, MSH6, PMS2, BRCA1, PALB2, and BARD1. In some embodiments, the composite biomarker score is incremented if the sample comprises the presence of a cancer-associated mutation in one or more DDR genes in the sample. In some embodiments, the composite biomarker score is incremented by 1 if the sample comprises the presence of a cancer-associated mutation in one or more DDR genes in the sample. In some embodiments, the composite biomarker score is incremented by 0 if the sample does not comprise the presence of a cancer-associated mutation in one or more DDR genes in the sample. In some embodiments, the composite biomarker score is not incremented if the sample does not comprise the presence of a cancer-associated mutation in one or more DDR genes in the sample.

In some embodiments, the cancer-associated mutation comprises one or more of a substitution of one or more nucleotides, an insertion of one or more nucleotides, or a deletion of one or more nucleotides in a DDR gene. In some embodiments, the cancer-associated mutation comprises one or more of a substitution of one or more nucleotides, an insertion of one or more nucleotides, or a deletion of one or more nucleotides in at least one DNA damage response (DDR) gene in the sample. In some embodiments, the at least one DDR gene is at least one of ATM, BRCA2, BRIP1, MRE11, POLE, MSH2, PARP1, MLH1, MSH6, PMS2, BRCA1, PALB2, and BARD1. In some embodiments, the at least one DDR gene includes each of ATM, BRCA2, BRIP1, MRE11, POLE, MSH2, PARP1, MLH1, MSH6, PMS2, BRCA1, PALB2, and BARD1. In some embodiments, the cancer-associated mutation in one or more DDR genes is an inactivating mutation, a loss-of-function mutation, a copy number alteration (e.g., a deletion), a rearrangement, or a gene fusion. In some embodiments, the cancer-associated mutation in one or more DDR genes results in an inactive polypeptide or protein. In some embodiments, the cancer-associated mutation is in at least ATM. In some embodiments, the cancer-associated mutation is in ATM. In some embodiments, the cancer-associated mutation is in at least BRCA2. In some embodiments, the cancer-associated mutation is in BRCA2. In some embodiments, the cancer-associated mutation is in at least BRIP1. In some embodiments, the cancer-associated mutation is in BRIP1. In some embodiments, the cancer-associated mutation is in at least MRE11. In some embodiments, the cancer-associated mutation is in MRE11. In some embodiments, the cancer-associated mutation is in at least POLE. In some embodiments, the cancer-associated mutation is in POLE. In some embodiments, the cancer-associated mutation is in at least MSH2. In some embodiments, the cancer-associated mutation is in MSH2. In some embodiments, the cancer-associated mutation is in at least PARP1. In some embodiments, the cancer-associated mutation is in PARP1. In some embodiments, the cancer-associated mutation is in at least MLH1. In some embodiments, the cancer-associated mutation is in at least MSH6. In some embodiments, the cancer-associated mutation is in at least PMS2. In some embodiments, the cancer-associated mutation is in at least BRCA1. In some embodiments, the cancer-associated mutation is in at least PALB2. In some embodiments, the cancer-associated mutation is in at least BARD1. In some embodiments, the cancer-associated mutation is in MLH1. In some embodiments, the cancer-associated mutation is in MSH6. In some embodiments, the cancer-associated mutation is in PMS2. In some embodiments, the cancer-associated mutation is in BRCA1. In some embodiments, the cancer-associated mutation is in PALB2. In some embodiments, the cancer-associated mutation is in BARD1.

In some embodiments, the cancer-associated mutation in the ATM gene results in an inactive polypeptide or protein encoded by ATM. In some embodiments, the cancer-associated mutation in ATM results in a polypeptide or protein encoded by ATM that has reduced activity, e.g., as compared to a polypeptide or protein encoded by an ATM gene without the cancer-associated mutation. In some embodiments, the cancer-associated mutation in ATM results in reduced expression of ATM. In some embodiments, the cancer-associated mutation in ATM results in loss of expression of ATM.

In some embodiments, the cancer-associated mutation in the BRCA2 gene results in an inactive polypeptide or protein encoded by BRCA2. In some embodiments, the cancer-associated mutation in BRCA2 results in a polypeptide or protein encoded by BRCA2 that has reduced activity, e.g., as compared to a polypeptide or protein encoded by an BRCA2 gene without the cancer-associated mutation. In some embodiments, the cancer-associated mutation in BRCA2 results in reduced expression of BRCA2. In some embodiments, the cancer-associated mutation in BRCA2 results in loss of expression of BRCA2.

In some embodiments, the cancer-associated mutation in the BRIP1 gene results in an inactive polypeptide or protein encoded by BRIP1. In some embodiments, the cancer-associated mutation in BRIP1 results in a polypeptide or protein encoded by BRIP1 that has reduced activity, e.g., as compared to a polypeptide or protein encoded by an BRIP1 gene without the cancer-associated mutation. In some embodiments, the cancer-associated mutation in BRIP1 results in reduced expression of BRIP1. In some embodiments, the cancer-associated mutation in BRIP1 results in loss of expression of BRIP1.

In some embodiments, the cancer-associated mutation in the MRE11 gene results in an inactive polypeptide or protein encoded by MRE11. In some embodiments, the cancer-associated mutation in MRE11 results in a polypeptide or protein encoded by MRE11 that has reduced activity, e.g., as compared to a polypeptide or protein encoded by an MRE11 gene without the cancer-associated mutation. In some embodiments, the cancer-associated mutation in MRE11 results in reduced expression of MRE11. In some embodiments, the cancer-associated mutation in MRE11 results in loss of expression of MRE11.

In some embodiments, the cancer-associated mutation in the POLE gene results in an inactive polypeptide or protein encoded by POLE. In some embodiments, the cancer-associated mutation in POLE results in a polypeptide or protein encoded by POLE that has reduced activity, e.g., as compared to a polypeptide or protein encoded by a POLE gene without the cancer-associated mutation. In some embodiments, the cancer-associated mutation in POLE results in reduced expression of POLE. In some embodiments, the cancer-associated mutation in POLE results in loss of expression of POLE.

In some embodiments, the cancer-associated mutation in the MSH2 gene results in an inactive polypeptide or protein encoded by MSH2. In some embodiments, the alteration in MSH2 results in a polypeptide or protein encoded by MSH2 that has reduced activity, e.g., as compared to a polypeptide or protein encoded by an MSH2 gene without the cancer-associated mutation. In some embodiments, the cancer-associated mutation in MSH2 results in reduced expression of MSH2. In some embodiments, the cancer-associated mutation in MSH2 results in loss of expression of MSH2.

In some embodiments, the cancer-associated mutation in the PARP1 gene results in an inactive polypeptide or protein encoded by PARP1. In some embodiments, the cancer-associated mutation in PARP1 results in a polypeptide or protein encoded by PARP1 that has reduced activity, e.g., as compared to a polypeptide or protein encoded by an PARP1 gene without the cancer-associated mutation. In some embodiments, the cancer-associated mutation in PARP1 results in reduced expression of PARP1. In some embodiments, the cancer-associated mutation in PARP1 results in loss of expression of PARP1.

In some embodiments, the cancer-associated mutation in the MLH1 gene results in an inactive polypeptide or protein encoded by MLH1. In some embodiments, the cancer-associated mutation in MLH1 results in a polypeptide or protein encoded by MLH1 that has reduced activity, e.g., as compared to a polypeptide or protein encoded by an MLH1 gene without the cancer-associated mutation. In some embodiments, the cancer-associated mutation in MLH1 results in reduced expression of MLH1. In some embodiments, the cancer-associated mutation in MLH1 results in loss of expression of MLH1.

In some embodiments, the cancer-associated mutation in the MSH6 gene results in an inactive polypeptide or protein encoded by MSH6. In some embodiments, the cancer-associated mutation in MSH6 results in a polypeptide or protein encoded by MSH6 that has reduced activity, e.g., as compared to a polypeptide or protein encoded by an MSH6 gene without the cancer-associated mutation. In some embodiments, the cancer-associated mutation in MSH6 results in reduced expression of MSH6. In some embodiments, the cancer-associated mutation in MSH6 results in loss of expression of MSH6.

In some embodiments, the cancer-associated mutation in the PMS2 gene results in an inactive polypeptide or protein encoded by PMS2. In some embodiments, the cancer-associated mutation in MSH6 results in a polypeptide or protein encoded by PMS2 that has reduced activity, e.g., as compared to a polypeptide or protein encoded by an PMS2 gene without the cancer-associated mutation. In some embodiments, the cancer-associated mutation in PMS2 results in reduced expression of PMS2. In some embodiments, the cancer-associated mutation in PMS2 results in loss of expression of PMS2.

In some embodiments, the cancer-associated mutation in the BRCA1 gene results in an inactive polypeptide or protein encoded by BRCA1. In some embodiments, the cancer-associated mutation in BRCA1 results in a polypeptide or protein encoded by BRCA1 that has reduced activity, e.g., as compared to a polypeptide or protein encoded by an BRCA1 gene without the cancer-associated mutation. In some embodiments, the cancer-associated mutation in BRCA1 results in reduced expression of BRCA1. In some embodiments, the cancer-associated mutation in BRCA1 results in loss of expression of BRCA1.

In some embodiments, the cancer-associated mutation in the PALB2 gene results in an inactive polypeptide or protein encoded by PALB2. In some embodiments, the cancer-associated mutation in PALB2 results in a polypeptide or protein encoded by PALB2 that has reduced activity, e.g., as compared to a polypeptide or protein encoded by an PALB2 gene without the cancer-associated mutation. In some embodiments, the cancer-associated mutation in PALB2 results in reduced expression of PALB2. In some embodiments, the cancer-associated mutation in PALB2 results in loss of expression of PALB2.

In some embodiments, the cancer-associated mutation in the BARD1 gene results in an inactive polypeptide or protein encoded by BARD1. In some embodiments, the cancer-associated mutation in BARD1 results in a polypeptide or protein encoded by BARD1 that has reduced activity, e.g., as compared to a polypeptide or protein encoded by an BARD1 gene without the cancer-associated mutation. In some embodiments, the cancer-associated mutation in BARD1 results in reduced expression of BARD1. In some embodiments, the cancer-associated mutation in BARD1 results in loss of expression of BARD1.

### 4. Loss of Heterozygosiky (LOH)

In some embodiments, the methods provided herein comprise determining a composite marker score, wherein the composite biomarker score is determined based on, at least in part, loss of heterozygosity in one or more human leukocyte antigen (HLA) genes. In some embodiments, loss of heterozygosity is assessed in one of the six HLA class I genes. In some embodiments, loss of heterozygosity is the loss of any one or more of the six HLA class I genes (HLA-A, HLA-B, HLA-C, HLA-DP, HLA-DQ, and/or HLA-DR). Loss of heterozygosity (LOH) may cause downregulated expression of HLA and provide tumor cells with an immune-escape tumor phenotype. Loss of heterozygosity (LOH) of the major HLA class I (HLA-I) genes, e.g., HLA-A, HLA-B, or HLA-C, is understood as a mechanism of immune evasion and has been found to be correlated with poorer outcomes in patients treated with immune checkpoint inhibitors. The LOH status of the gene is determined based on sequence read data derived from sequencing the sample from the subject. In such embodiments, the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique.

In some embodiments, the composite biomarker score is incremented if LOH is detected in one or more of the HLA genes. In some embodiments, the composite biomarker score is incremented if one or more of the HLA genes is determined to exhibit high LOH.

### G. Determination of a Composite Biomarker

In some embodiments, the composite biomarker score is determined based on, at least in part, a tumor mutational burden (TMB) score. In some embodiments, the composite biomarker score is determined based on, at least in part, the percent of cells in a tumor sample that are positive for PD-L1. In some embodiments, the composite biomarker score is determined based on the tumor sample. In some embodiments, ARID1A is inactivated or reduced in the tumor sample. In some embodiments, the KEAP1/NFE2L2 pathway is inactivated or reduced in the tumor sample. In some embodiments, STK11/LKB1 is inactivated or reduced in the tumor sample. In some embodiments, CDKN2A is inactivated or reduced in the tumor sample. In some embodiments, the DDR pathway is inactivated or reduced. Reduction of a pathway or gene may be assessed, for example, by measuring gene expression or protein function, which may be in comparison with normal tissue or other suitable controls.

In some embodiments, the composite biomarker score is determined based on, at least in part, a tumor mutational burden (TMB) score. In some embodiments, the composite biomarker score is determined based on, at least in part, the percent of cells in a tumor sample that are positive for PD-L1. In some embodiments, the composite biomarker score is determined based on, at least in part, the presence of a cancer-associated mutation in a ARID1A gene in a tumor sample. In some embodiments, the composite biomarker score is determined based on, at least in part, the presence of a cancer-associated mutation in a KEAP1/NFE2L2 pathway gene in the sample. In some embodiments, the composite biomarker score is determined based on, at least in part, the presence of a cancer-associated mutation in a STK11/LKB1 gene in a tumor sample. In some embodiments, the composite biomarker score is determined based on, at least in part, the presence of a cancer-associated mutation in a CDKN2A gene in a tumor sample. In some embodiments, the composite biomarker score is determined based on, at least in part, the presence of a cancer-associated mutation in a DNA damage response (DDR) gene (such as the DDR genes ATM, BRCA2, BRIP1, MRE11, POLE, MSH2, PARP1, MLH1, MSH6, PMS2, BRCA1, PALB2, and BARD1) in a tumor sample.

In some embodiments, the composite biomarker score is determined, in part, by the tumor mutational burden (TMB) score of a tumor sample associated with the cancer, the percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene in the sample; and determining, based on the determined composite biomarker score, whether the subject will likely benefit from the treatment comprising the immune checkpoint inhibitor. In some embodiments, the composite biomarker score is further based on the (h) status of one or more human leukocyte antigen (HLA) genes of the cancer are determined to exhibit loss of heterozygosity (LOH). In some embodiments, the one or more HLA genes comprise at least HLA-A, HLA-B, and HLA-C.

In some embodiments, the composite biomarker score is determined based on at least tumor mutational burden (TMB) and the percent of cells in a tumor sample that are positive for PD-L1. In some embodiments, the composite biomarker score is also determined based on at least one of the following biomarkers: the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene in the sample; and determining, based on the determined composite biomarker score, whether the subject will likely benefit from the treatment comprising the immune checkpoint inhibitor.

In some embodiments, the composite biomarker score is determined based on at least three of the following individual biomarkers: tumor mutational burden (TMB), percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene in the sample; and determining, based on the determined composite biomarker score, whether the subject will likely benefit from the treatment comprising the immune checkpoint inhibitor. In some embodiments, the methods further comprise determining the composite biomarker score based on the status of one or more human leukocyte antigen (HLA) genes of the cancer are determined to exhibit loss of heterozygosity (LOH).

In some embodiments, the composite biomarker score is determined based on at least four of the following individual biomarkers: tumor mutational burden (TMB), percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene in the sample; and determining, based on the determined composite biomarker score, whether the subject will likely benefit from the treatment comprising the immune checkpoint inhibitor. In some embodiments, the methods further comprise determining the composite biomarker score based on the status of one or more human leukocyte antigen (HLA) genes of the cancer are determined to exhibit loss of heterozygosity (LOH).

In some embodiments, the composite biomarker score is determined based on at least five of the following individual biomarkers: tumor mutational burden (TMB), percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene in the sample; and determining, based on the determined composite biomarker score, whether the subject will likely benefit from the treatment comprising the immune checkpoint inhibitor. In some embodiments, the methods further comprise determining the composite biomarker score based on the status of one or more human leukocyte antigen (HLA) genes of the cancer are determined to exhibit loss of heterozygosity (LOH).

In some embodiments, the composite biomarker score is determined based on at least six of the following individual biomarkers: tumor mutational burden (TMB), percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene in the sample; and determining, based on the determined composite biomarker score, whether the subject will likely benefit from the treatment comprising the immune checkpoint inhibitor. In some embodiments, the methods further comprise determining the composite biomarker score based on the status of one or more human leukocyte antigen (HLA) genes of the cancer are determined to exhibit loss of heterozygosity (LOH).

In some embodiments, the composite biomarker score is determined based on at least seven of the following individual biomarkers: tumor mutational burden (TMB), percent of cells in the tumor sample that are positive for PD-L1, the presence of a cancer-associated mutation in a ARID1A gene in the sample, the presence of a cancer-associated mutation in a KEAP1/NFE2L2 pathway gene in the sample, the presence of a cancer-associated mutation in a STK11/LKB1 gene in the sample, the presence of a cancer-associated mutation in a CDKN2A gene in the sample, and the presence of a cancer-associated mutation in a DNA damage response (DDR) gene in the sample; and determining, based on the determined composite biomarker score, whether the subject will likely benefit from the treatment comprising the immune checkpoint inhibitor. In some embodiments, the methods further comprise determining the composite biomarker score based on the status of one or more human leukocyte antigen (HLA) genes of the cancer are determined to exhibit loss of heterozygosity (LOH).

In some embodiments, the composite biomarker score is calculated by adding to the score for each of biomarkers that are met. In some embodiments, the composite biomarker score starts at 0. In some embodiments, the determination of whether a subject will likely benefit from the treatment comprising the immune checkpoint inhibitor is based on the composite biomarker score being at least a threshold value. In some embodiments, if the composite biomarker score is less than the threshold value, the subject is identified as one who will not benefit from treatment with an immune checkpoint inhibitor. In some embodiments, if the composite biomarker score is less than the threshold value, the subject is identified as one who will not benefit from a monotherapy treatment with an immune checkpoint inhibitor. In some embodiments, the threshold value is 2. In some embodiments, the threshold value is 3. In some embodiments, the threshold value is 4. In some embodiments, the threshold value is 5. In some embodiments, the threshold value is 6. In some embodiments, the threshold value is 7. In some embodiments, the subject is human.

In some embodiments, the composite biomarker score being at least a threshold value has a predictive value of whether the patient will benefit from the treatment comprising an immune checkpoint inhibitor. In some embodiments, the threshold value is 5. In some embodiments, the threshold value is 6. In some embodiments, the threshold value is 7.

In some embodiments, the methods disclosed further comprise characterizing the subject as having a likelihood of benefitting from the treatment comprising an immune checkpoint inhibitor, wherein the subject is characterized based on the determined biomarker score. In some embodiments, the methods disclosed further comprise characterizing the subject as having a low likelihood of benefitting from the treatment comprising an immune checkpoint inhibitor, an indeterminant likelihood (or a medium likelihood) of benefitting from the treatment comprising an immune checkpoint inhibitor, or having a high likelihood of benefitting from the treatment comprising the immune checkpoint inhibitor, wherein the subject is characterized based on the determined biomarker score.

In some embodiments, the threshold for having a high likelihood of benefitting from the treatment comprising the immune checkpoint inhibitor comprises a range composite biomarker score values. In some embodiments, the threshold value is at least 5. In some embodiments, the threshold value is 5-8. In some embodiments, a binned composite biomarker score of 5 or higher indicates that the subject has a high likelihood of benefitting from the treatment comprising an immune checkpoint inhibitor. In some embodiments, the threshold for having an indeterminate likelihood (or medium likelihood) of benefitting from the treatment comprising the immune checkpoint inhibitor comprises a range composite biomarker score values. In some embodiments, the threshold value is at least 3. In some embodiments, the threshold value is 3-4. In some embodiments, a binned composite biomarker score of 3-4 indicates that the subject has an indeterminate likelihood (or medium likelihood) of benefitting from the treatment comprising an immune checkpoint inhibitor. In some embodiments, the threshold for having a low likelihood of benefitting from the treatment comprising the immune checkpoint inhibitor comprises a range composite biomarker score values. In some embodiments, the threshold value is 2 or less. In some embodiments, the threshold value is 0-2. In some embodiments, a binned composite biomarker score of 2 or lower indicates that the subject has a low likelihood of benefitting from the treatment comprising an immune checkpoint inhibitor. In some embodiments, the immune checkpoint inhibitor is an anti-PD-L1 antibody. In some embodiments, the immune checkpoint inhibitor is atezolizumab, avelumab, or durvalumab. In some embodiments, the immune checkpoint inhibitor is atezolizumab. In some embodiments, the immune checkpoint inhibitor is a CTLA-4 inhibitor. In some embodiments, the immune checkpoint inhibitor is ipilimumab. In some embodiments, the subject is human. In some embodiments, the benefit is improved clinical benefit. In some embodiments, the benefit is overall survival. In some embodiment, the benefit is median survival.

In some embodiments, the composite biomarker score is calculated by adding to the score for some of biomarkers that are met, and decreasing the score for some of the biomarkers that are met. In some embodiments, the composite biomarker score starts at 0. In some embodiments, the determination of whether a subject will likely benefit from the treatment comprising the immune checkpoint inhibitor is based on the composite biomarker score being at least a threshold value. In some embodiments, if the composite biomarker score is less than the threshold value, the subject is identified as one who will not benefit from treatment with an immune checkpoint inhibitor. In some embodiments, if the composite biomarker score is less than the threshold value, the subject is identified as one who will not benefit from a monotherapy treatment with an immune checkpoint inhibitor. In some embodiments, the threshold value is −1 or below. In some embodiments, the threshold value is −1. In some embodiments, the threshold value is between 0 and 2. In some embodiments, the threshold value is 1. In some embodiments, the threshold value is 2. In some embodiments, the threshold value is 3 or above. In some embodiments, the threshold value is 3. In some embodiments, the threshold value is 4. In some embodiments, the threshold value is 5. In some embodiments, the threshold value is 6. In some embodiments, the threshold value is 7. In some embodiments, the subject is human.

In some embodiments, the composite biomarker score being at least a threshold value has a predictive value of whether the patient will benefit from the treatment comprising an immune checkpoint inhibitor. In some embodiments, the threshold value is 3 or above. In some embodiments, the threshold value is 3. In some embodiments, the threshold value is 4. In some embodiments, the threshold value is 5. In some embodiments, the threshold value is 6. In some embodiments, the threshold value is 7. In some embodiments, the subject is human.

In some embodiments, the methods disclosed further comprise characterizing the subject as having a likelihood of benefitting from the treatment comprising an immune checkpoint inhibitor, wherein the subject is characterized based on the determined biomarker score. In some embodiments, the methods disclosed further comprise characterizing the subject as having a low likelihood of benefitting from the treatment comprising an immune checkpoint inhibitor, an indeterminant likelihood (or a medium likelihood) of benefitting from the treatment comprising an immune checkpoint inhibitor, or having a high likelihood of benefitting from the treatment comprising the immune checkpoint inhibitor, wherein the subject is characterized based on the determined biomarker score.

In some embodiments, the threshold for having a high likelihood of benefitting from the treatment comprising the immune checkpoint inhibitor comprises a range composite biomarker score values. In some embodiments, the threshold value is at least 3. In some embodiments, the threshold value is 3 or above. In some embodiments, a binned composite biomarker score of 3 or higher indicates that the subject has a high likelihood of benefitting from the treatment comprising an immune checkpoint inhibitor. In some embodiments, the threshold for having an indeterminate likelihood (or medium likelihood) of benefitting from the treatment comprising the immune checkpoint inhibitor comprises a range composite biomarker score values. In some embodiments, the threshold value is between 0-2. In some embodiments, the threshold value is at least 1. In some embodiments, the threshold value is at least 2. In some embodiments, a binned composite biomarker score of 0-2 indicates that the subject has an indeterminate likelihood (or medium likelihood) of benefitting from the treatment comprising an immune checkpoint inhibitor. In some embodiments, the threshold for having a low likelihood of benefitting from the treatment comprising the immune checkpoint inhibitor comprises a range composite biomarker score values. In some embodiments, the threshold value is −1 or less. In some embodiments, the threshold value is −1. In some embodiments, the threshold value is −2. In some embodiments, the threshold value is −3. In some embodiments, a binned composite biomarker score of −1 or lower indicates that the subject has a low likelihood of benefitting from the treatment comprising an immune checkpoint inhibitor. In some embodiments, the immune checkpoint inhibitor is an anti-PD-L1 antibody. In some embodiments, the immune checkpoint inhibitor is atezolizumab, avelumab, or durvalumab. In some embodiments, the immune checkpoint inhibitor is atezolizumab. In some embodiments, the immune checkpoint inhibitor is a CTLA-4 inhibitor. In some embodiments, the immune checkpoint inhibitor is ipilimumab. In some embodiments, the subject is human. In some embodiments, the benefit is improved clinical benefit. In some embodiments, the benefit is overall survival. In some embodiment, the benefit is median survival.

In some embodiments, the methods disclosed comprise creating a record associated with the subject designating the subject as (i) one who will likely benefit from the treatment comprising an immune checkpoint inhibitor or (ii) one who will likely not benefit from the treatment comprising an immune checkpoint inhibitor, wherein the designation is based on the composite biomarker score. In some embodiments, the subject is human. In some embodiments, the immune checkpoint inhibitor is an anti-PD-L1 antibody. In some embodiments, the immune checkpoint inhibitor is atezolizumab, avelumab, or durvalumab. In some embodiments, the immune checkpoint inhibitor is atezolizumab. In some embodiments, the immune checkpoint inhibitor is a CTLA-4 inhibitor. In some embodiments, the immune checkpoint inhibitor is ipilimumab. In some embodiments, the subject is human. In some embodiments, the benefit is improved clinical benefit. In some embodiments, the benefit is overall survival. In some embodiment, the benefit is median survival.

In some embodiments, the methods disclosed comprise treating a subject having a cancer, wherein the subject is identified as one who will benefit from an immune checkpoint inhibitor according, the method comprising administering the immune checkpoint inhibitor to the subject. In some embodiments, the immune checkpoint inhibitor is an anti-PD-L1 antibody. In some embodiments, the immune checkpoint inhibitor is atezolizumab, avelumab, or durvalumab. In some embodiments, the immune checkpoint inhibitor is atezolizumab. In some embodiments, the immune checkpoint inhibitor is a CTLA-4 inhibitor. In some embodiments, the immune checkpoint inhibitor is ipilimumab. In some embodiments, the subject is human. In some embodiments, the benefit is improved clinical benefit. In some embodiments, the benefit is overall survival. In some embodiment, the benefit is median survival.

### H. REPORTS

The composite biomarker score described herein may be recorded in a report. The report generated is associated with a subject and may include identifying information of the subject. In some embodiments, the report includes the composite biomarker score. In some embodiments, the report includes a therapeutic recommendation or prognostic prediction based on the composite biomarker score. For example, if the composite biomarker score is at least a threshold value, indicating the subject is a candidate for a therapy with an immune checkpoint inhibitor, in an exemplary embodiment, the report would contain information identifying the patient (such as name, sex, date of birth, and cancer diagnosis), the composite biomarker score, and an indication that the patient is a candidate for therapy with an immune checkpoint inhibitor (such as an ICI monotherapy regimen). In contrast, in an exemplary embodiment, if the composite biomarker score is below a threshold value, indicating the subject is not a candidate for a therapy with an ICI, the report would contain information identifying the patient, the composite biomarker score, and an indication that the patient is not a candidate for therapy with an ICI, that the ICI should be combined with another anti-cancer therapy, or that the patient is a candidate for a non-ICI therapy. In some embodiments, the report does not contain the composite biomarker score, but would contain information that was generated based on the composite biomarker score (such as a therapeutic recommendation or a prognostic statement). In some embodiments, the methods provided herein comprise generating a report. In some embodiments, the methods provided herein comprise receiving a report. For example, in some embodiments, a method of treatment comprises receiving a report as described herein, which was generated based on the determined composite biomarker score, and then, based on the report, treating the subject. In some embodiments, the methods provided herein comprise providing a report to a party (such as a subject's caregiver or the subject).

In some embodiments, a report according to the present disclosure comprises information about one or more of the assessed biomarkers which are components of the composite biomarker score. In some embodiments, the report comprises information about at least one assessed biomarker. In some embodiments, the report comprises information about at least three, at least 4, at least 5, at least 6, or at least 7 of the assessed biomarkers. In some embodiments, the report comprises information about the composite biomarker score. In some embodiments, the composite biomarker score starts at 0.

In some embodiments, one of the assessed biomarkers is a tumor mutational burden (TMB) score. In some embodiments, one of the assessed biomarkers is the percent of cells in a tumor sample that are positive for PD-L1. In some embodiments, the one of the assessed biomarkers is the presence of a cancer-associated mutation in a ARID1A gene in a tumor sample. In some embodiments, one of the assessed biomarkers is the presence of a cancer-associated mutation in a KEAP1/NFE2L2 pathway gene in the sample. In some embodiments, one of the assessed biomarkers is the presence of a cancer-associated mutation in a STK11/LKB1 gene in a tumor sample. In some embodiments, one of the assessed biomarkers is the presence of a cancer-associated mutation in a CDKN2A gene in a tumor sample. In some embodiments, one of the assessed biomarkers is a cancer-associated mutation in a DNA damage response (DDR) gene (such as the DDR genes including one or more of ATM, BRCA2, BRIP1, MRE11, POLE, MSH2, PARP1, MLH1, MSH6, PMS2, BRCA1, PALB2, and BARD1) in a tumor sample.

In some embodiments, the report comprises a determination of whether the subject will likely benefit from or respond to the treatment comprising the immune checkpoint inhibitor, based on the composite biomarker score being at least a threshold value. In some embodiments, the determination is a high likelihood that the subject will benefit from or respond to the treatment. In some embodiments, the determination is an indeterminate or medium likelihood that the subject will benefit from or respond to the treatment. In some embodiments, the determination is a low likelihood that the subject will benefit from or respond to the treatment.

In some embodiments, the report contains the cancer that the subject has, had, or is suspected to have. In some embodiments, the report comprises a treatment, a therapy, or one or more treatment options for a subject having a cancer of the disclosure. In some embodiments, the therapy is immune checkpoint inhibitor therapy, such as if the composite biomarker score is at least a threshold value. In some embodiments, the therapy is a non-ICI therapy, such as if the composite biomarker score is below a threshold value. In some embodiments, the therapy is an immune checkpoint inhibitor therapy in combination with an additional anti-cancer agent, such as if the composite biomarker score is below a threshold value (indicating that the ICI therapy will not be effective as a monotherapy).

In some embodiments, the immune checkpoint inhibitor comprises one or more of atezolizumab, avelumab, or durvalumab. In some embodiments, the immune checkpoint inhibitor is atezolizumab. In some embodiments, the immune checkpoint therapy is a PD-L1 inhibitor. In some embodiments, the immune checkpoint therapy is a CTLA-4 inhibitor. In some embodiments, the immune checkpoint inhibitor comprises ipilimumab.

In some embodiments, a report according to the present disclosure comprises information about a composite biomarker score from a sample obtained from a subject, such as a subject having a cancer. In some embodiments, the cancer is NSCLC. In some embodiments, the report comprises an identifier for the subject from which the sample was obtained.

In some embodiments, the report includes information on the role of a biomarker in disease, such as in cancer. In some embodiments, the cancer is NSCLC. Such information can include one or more of: information on prognosis of a cancer; or information on therapeutic options that should be avoided. In some embodiments, the report includes information on the likely effectiveness, acceptability, and/or advisability of applying a therapeutic option to a subject having a cancer and identified in the report. In some embodiments, the report includes information or a recommendation on the administration of a treatment (e.g., an anti-cancer therapy provided herein, such as an immune checkpoint inhibitor, or a treatment selected or identified according to the methods provided herein). In some embodiments, the information or recommendation includes the dosage of the treatment and/or a treatment regimen (e.g., as a monotherapy, or in combination with other treatments, such as a second anti-cancer agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from a subject, e.g., a subject having a cancer, e.g., a squamous cell cancer or NSCLC; detecting a biomarker or one or more biomarkers in the sample, or acquiring knowledge of the presence of one or more biomarkers; and generating a report. In some embodiments, a report generated according to the methods provided herein comprises one or more of: information about the presence or absence one or more biomarkers; an identifier for the subject from which the sample was obtained; information on the role of the one or more biomarkers in disease (e.g., such as the role of the tumor mutational burden (TMB) score of a tumor sample associated with the cancer; the percent of cells in the tumor sample that are positive for PD-L1; the presence of a cancer-associated mutation in a ARID1A gene in the sample; the presence of a cancer-associated mutation in a KEAP1/NFE2L2 pathway gene in the sample; the presence of a cancer-associated mutation in a STK11/LKB1 pathway gene in the sample; the presence of a cancer-associated mutation in a CDKN2A gene in the sample; and the presence of a cancer-associated mutation in a DNA damage response (DDR) gene in the sample, the biomarker also comprises the status of one or more human leukocyte antigen (HLA) genes of the cancer are determined to exhibit loss of heterozygosity (LOH) in cancer); information on prognosis, resistance, or potential or suggested therapeutic options (e.g., an anti-cancer therapy provided herein, such as an immune checkpoint inhibitor, or a treatment selected or identified according to the methods provided herein); information on the likely effectiveness, acceptability, or the advisability of applying a therapeutic option (e.g., an anti-cancer therapy provided herein, such as an immune checkpoint inhibitor, or a treatment selected or identified according to the methods provided herein) to the individual; a recommendation or information on the administration of a treatment (e.g., an anti-cancer therapy provided herein, such as an immune checkpoint inhibitor, or a treatment selected or identified according to the methods provided herein); or a recommendation or information on the dosage or treatment regimen of a treatment (e.g., an anti-cancer therapy provided herein, such as an immune checkpoint inhibitor, or a treatment selected or identified according to the methods provided herein), e.g., in combination with other treatments (e.g., a second anti-cancer therapy). In some embodiments, the report generated is a personalized cancer report.

A report according to the present disclosure may be in an electronic, web-based, or paper form. The report may be provided to a subject or a patient, or to an individual or entity other than the subject or patient (e.g., other than the subject or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from a subject (e.g., a subject having a cancer). In some embodiments, the report is provided or delivered to an individual or entity within any of about 1 day or more, about 7 days or more, about 14 days or more, about 21 days or more, about 30 days or more, about 45 days or more, or about 60 days or more from determining a composite biomarker score. In some embodiments, the report is provided or delivered to an individual or entity within any of about 1 day or more, about 7 days or more, about 14 days or more, about 21 days or more, about 30 days or more, about 45 days or more, or about 60 days or more from determining the tumor mutational burden (TMB) score of a tumor sample associated with the cell. In some embodiments, the report is provided or delivered to an individual or entity within any of about 1 day or more, about 7 days or more, about 14 days or more, about 21 days or more, about 30 days or more, about 45 days or more, or about 60 days or more from determining the percent of cells in the tumor sample that are positive for PD-L1. In some embodiments, the report is provided or delivered to an individual or entity within any of about 1 day or more, about 7 days or more, about 14 days or more, about 21 days or more, about 30 days or more, about 45 days or more, or about 60 days or more from determining the presence of a cancer-associated mutation in the ARID1A gene in the sample. In some embodiments, the report is provided or delivered to an individual or entity within any of about 1 day or more, about 7 days or more, about 14 days or more, about 21 days or more, about 30 days or more, about 45 days or more, or about 60 days or more from determining the presence of a cancer-associated mutation in a KEAP1/NFEL2 pathway gene in the sample. In some embodiments, the report is provided or delivered to an individual or entity within any of about 1 day or more, about 7 days or more, about 14 days or more, about 21 days or more, about 30 days or more, about 45 days or more, or about 60 days or more from determining the presence of a cancer-associated mutation in the STK11/LKB1 gene in the sample. In some embodiments, the report is provided or delivered to an individual or entity within any of about 1 day or more, about 7 days or more, about 14 days or more, about 21 days or more, about 30 days or more, about 45 days or more, or about 60 days or more from determining the presence of a cancer-associated mutation in the CDKN2A gene in the sample. In some embodiments, the report is provided or delivered to an individual or entity within any of about 1 day or more, about 7 days or more, about 14 days or more, about 21 days or more, about 30 days or more, about 45 days or more, or about 60 days or more from determining the presence of a cancer-associated mutation in a DDR gene in the sample. In some embodiments, the DDR gene is ATM, BRCA2, BRIP1, MRE11, POLE, MSH2, and PARP1. In some embodiments, the report is provided or delivered to an individual or entity within any of about 1 day or more, about 7 days or more, about 14 days or more, about 21 days or more, about 30 days or more, about 45 days or more, or about 60 days or more from determining the LOH status of one or more HLA genes.

### I. IMMUNE CHECKPOINT INHIBITORS

The methods described herein pertain, in certain embodiments, to means of predicting the efficacy of an immune checkpoint inhibitor (ICI) therapy and/or administering an ICI to an individual having a cancer. In some instances, the method can further include administering an immune checkpoint inhibitor or applying an immune checkpoint inhibitor to the subject based on the composite biomarker score. In some embodiments, the efficacy of the ICI is predicted as a first line treatment. In some embodiments, the ICI is administered as a first line therapy for a cancer. In some embodiments, the ICI is the only treatment administered or indicated. In some embodiments, the ICI therapy consists of a single active agent.

As is known in the art, a checkpoint inhibitor targets at least one immune checkpoint protein to alter the regulation of an immune response. Immune checkpoint proteins include, e.g., CTLA-4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA-4 antagonist (e.g., an anti-CTLA-4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments according to any of the embodiments described herein, the immune checkpoint inhibitor comprises a PD-1 antagonist/inhibitor or a PD-L1 antagonist/inhibitor.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist/inhibitor is a molecule that inhibits the binding of PD-1 to its ligand binding partners. In a specific embodiment, the PD-1 ligand binding partners are PD-L1 and/or PD-L2. In another instance, a PD-L1 binding antagonist/inhibitor is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is MDX-1 106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IB1I308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist/inhibitor of CTLA-4. In some embodiments, the checkpoint inhibitor is a small molecule antagonist of CTLA-4. In some embodiments, the checkpoint inhibitor is an anti-CTLA-4 antibody. CTLA-4 is part of the CD28-B7 immunoglobulin superfamily of immune checkpoint molecules that acts to negatively regulate T cell activation, particularly CD28-dependent T cell responses. CTLA-4 competes for binding to common ligands with CD28, such as CD80 (B7-1) and CD86 (B7-2), and binds to these ligands with higher affinity than CD28. Blocking CTLA-4 activity (e.g., using an anti-CTLA-4 antibody) is thought to enhance CD28-mediated costimulation (leading to increased T cell activation/priming), affect T cell development, and/or deplete Tregs (such as intratumoral Tregs). In some embodiments, the CTLA-4 antagonist is a small molecule, a nucleic acid, a polypeptide (e.g., antibody), a carbohydrate, a lipid, a metal, or a toxin. In some embodiments, the CTLA-4 inhibitor comprises ipilimumab (1B1310, BMS-734016, MDX010, MDX-CTLA-4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the immune checkpoint inhibitor is monovalent and/or monospecific. In some embodiments, the immune checkpoint inhibitor is multivalent and/or multispecific.

In some embodiments, the immune checkpoint inhibitor may be administered in combination with an immunoregulatory molecule or a cytokine. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, I309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is a first line immune checkpoint inhibitor. In some embodiments, the immune checkpoint inhibitor is a second line immune checkpoint inhibitor. In some embodiments, an immune checkpoint inhibitor is administered in combination with one or more additional anti-cancer therapies or treatments. In some embodiments, the immune checkpoint inhibitor is administered after an anti-cancer therapy. In some embodiments, the anti-cancer therapy is platinum therapy.

### J. TREATMENTS AND TREATMENT EFFECTS

The methods described herein provide improved therapies and/or therapeutic effects. The improved therapies and/or therapeutic effects are based, in part, on identifying, predicting, or stratification of individuals with cancer a composite biomarker score above or below a threshold score. Once a composite biomarker score is determined the individuals can receive appropriate therapies, leading to improved clinical outcomes including improved survival (such as improved progression-free survival and/or improved overall survival). The individuals may be any of the individuals described in the preceding sections. In some embodiments, the methods disclosed comprise treating a subject having a cancer, wherein the subject is identified as one who will benefit from an immune checkpoint inhibitor according, the method comprising administering the immune checkpoint inhibitor to the subject.

Accordingly, in some embodiments, the methods comprise administering an immune checkpoint inhibitor (ICI, such as an ICI described in Section VII) if a composite biomarker score in a sample from an individual having a cancer is at least a threshold score. In some embodiments, the methods comprise administering an ICI therapy (such as an ICI described in Section V) if a sample from the individual is assessed to meet the threshold value. In some embodiments, the methods comprise administering a chemotherapy if a sample from the individual is assessed to not meet the threshold value. In some embodiments, the chemotherapy is docetaxel. In some embodiments, the threshold value is 5 or above. In some embodiments, the threshold value is 5.

In some embodiments, the methods of treatment described herein provide a clinical benefit and/or an improved clinical benefit for individuals having a cancer. In some embodiments, the methods provide improved clinical benefit when compared to an alternative therapy. For example, in some embodiments, the methods comprise administering an ICI, wherein the individual will, or is expected to, benefit from the ICI therapy as compared to treatment with a chemotherapy regimen. In some embodiments, the methods comprise administering a chemotherapy regimen, where the individual will, or is expected to, benefit from the chemotherapy regimen as compared to treatment with an ICI therapy.

In some embodiments, the clinical benefit is improved survival (such as improved PFS and/or improved OS). In some embodiments, the treatment improves PFS by at least one month, such as any of at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 2 years, at least about 3 years, at least about 4 years, or more, after administration. In some embodiments, the treatment improves OS by at least one month, such as any of at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 2 years, at least about 3 years, at least about 4 years, or more, after administration. In some embodiments, the treatment methods provide improved objective response rate of at least 20%, such as any of about 25%, about 30%, about 35%, about 40%, about 45%, about 50%, about 55%, about 60%, about 65%, about 70%, about 75%, about 80%, about 85%, about 90%, about 95%, or about 100%.

In one or more embodiments of the methods described above, the survival is a progression-free survival, an overall survival, a disease-free survival (DFS), an objective response rate (ORR), a time to tumor progression (TTP), a time to treatment failure (TTF), a durable complete response (DCR), or a time to next treatment (TTNT).

### K. CANCERS TO BE ASSESSED OR TREATED

The methods described herein pertain to individuals having a cancer and assessment of the cancers (by assessment of a sample, such as a blood sample or a tumor biopsy sample) to identify suitable treatments for the individual. Exemplary cancers to be treated or assessed include, but are not limited to, a B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like. In some embodiments, the cancer is a NSCLC, colorectal cancer, cholangiocarcinoma, breast cancer, stomach cancer, melanoma, pancreatic cancer, prostate cancer, ovarian cancer, esophageal cancer, gastro-esophageal cancer, or a cancer of unknown primary. In some embodiments, the cancer is metastatic urothelial carcinoma. In some embodiments, the cancer is metastatic gastric adenocarcinoma. In some embodiments, the cancer is breast cancer. In some embodiments, the cancer is metastatic endometrial cancer. In some embodiments, the cancer is prostate cancer. In some embodiments, the cancer is castration resistant prostate cancer. In some embodiments, the cancer is colorectal cancer. In some embodiments, the cancer is lung cancer. In some embodiments, the cancer is melanoma. In some embodiments, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm. Premaligancy, as used herein, refers to a tissue that is not yet malignant but is poised to become malignant.

In some embodiments, the cancer to be treated has never been treated with an anti-cancer therapy. In some embodiments, the cancer to be treated or assessed has never been treated, or is not currently being treated, with a chemotherapy regimen. In some embodiments, the cancer to be treated or assessed has previously been treated with an anti-cancer therapy. In some embodiments, the cancer to be treated or assessed has previously been treated with a chemotherapy regimen. In some embodiments, the chemotherapy is a platinum-based therapy.

In some embodiments, the cancer to be treated is in a subject with the cancer, wherein it has been determined whether the subject will likely benefit from the treatment comprising the immune checkpoint inhibitor based on the composite biomarker score being at least a threshold value. In some embodiments, the threshold value is 5 or greater. In some embodiments, the threshold value is 5. In some embodiments, the cancer to be treated is NSCLC.

### EXAMPLES

The following examples are included for illustrative purposes only and are not intended to limit the scope of the invention.

### L. Example 1: Composite Biomarker Scores as Predictors of Treatment Outcome

This Example shows that integrating single biomarkers into a composite biomarker is useful in identifying NSCLC patients that are likely to respond to immunotherapy with an immune checkpoint inhibitor versus those with primary resistance to the immunotherapy.

### Methods

A phase III trial that randomized 1250 patients with advanced stage non-small cell lung carcinoma (NSCLC) was selected. The patients who had received one or more platinum based combination therapies received atezolizumab (atezo) or docetaxel. Atezolizumab was given as an intravenous 1200 mg fixed dose every 3 weeks, and docetaxel was given intravenously at 75 mg/m² every 3 weeks. Treatment was administered until unacceptable toxicity or disease progression, as assessed by the investigator. Patient response was evaluated in accordance to the RECIST (Response Evaluation Criteria in Solid Tumors, version 1.1) response criteria. Patients identified as having complete or partial response were categorized as a responder and patients identified as having progressive or stable disease were categorized as a non-responder.

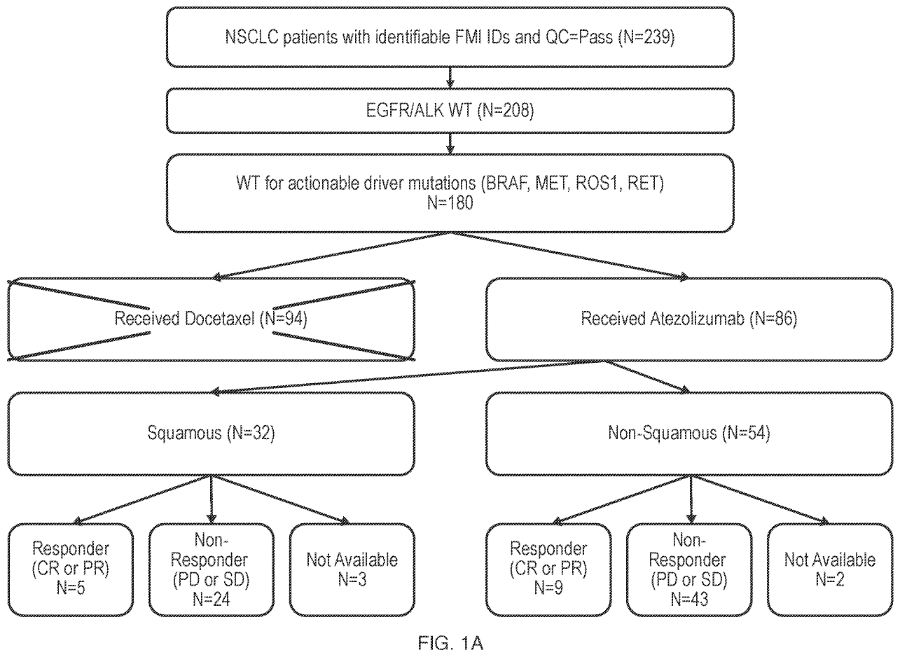

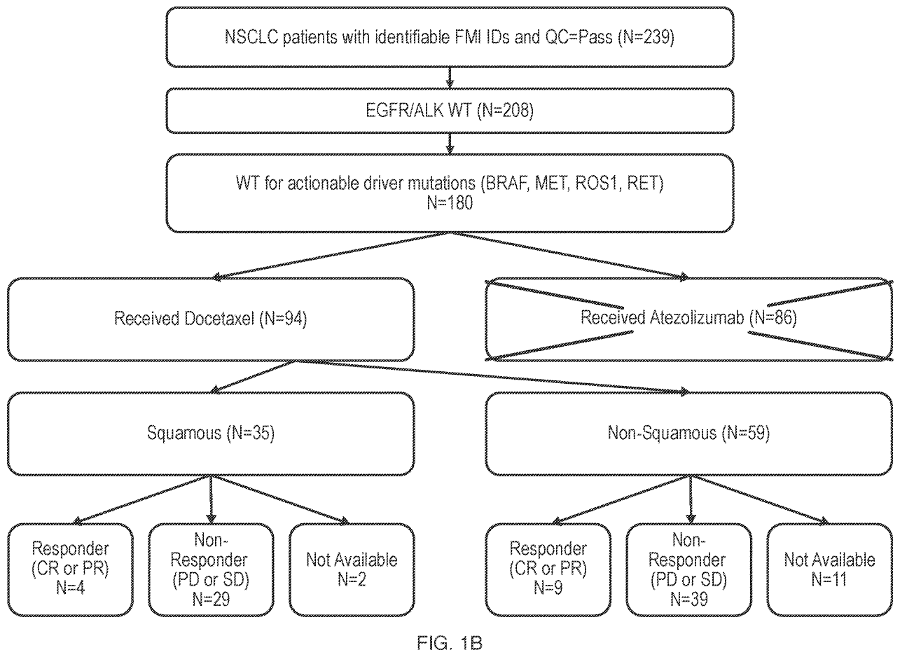

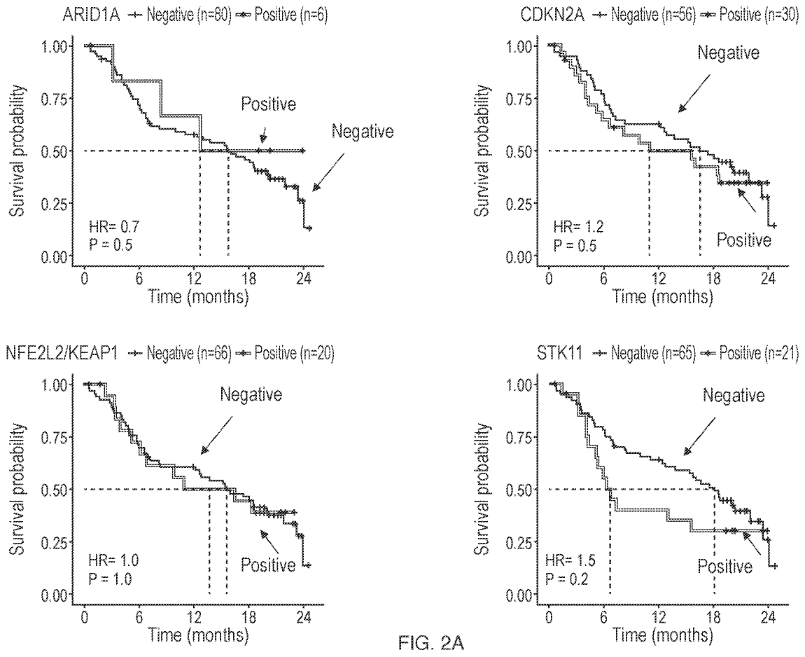

Of these patients, 239 participants were profiled using a tissue based comprehensive genomic profiling test and were selected for further analysis. 180 individuals were wild-type for EGFR, ALK, ROS1, BRAF, MET and RET. 86 received Atezo while 94 received docetaxel (FIG. 1A-B).

7 individual biomarkers were identified and the hazard ratios were determined for individuals who were either positive or negative for the biomarker (Table E1). A nominal system was subsequently identified, wherein composite scores were based on individual biomarker positivity and further categorized into levels (composite biomarker score of 0-2 [low]; biomarker score of 3-4 [medium]; biomarker score of 5-8 [high]). A Cox proportional hazards regression model was then used to investigate the effect of individual covariates and three composite bins on overall survival of the two treatment arms.

TABLE E1
Components included in the composite biomarker score

| Biomarker | Status | Score |
|---|---|---|
| TMB (tumor | <10 mut/Mb | 0 |
| mutational | | |
| burden) | | |
| TMB (tumor | =10 to less than 20 mut/Mb | 1 |
| mutational | | |
| burden) | | |
| TMB (tumor | ≥20 mut/Mb | 2 |
| mutational | | |
| burden) | | |
| PD-L1 | IHC <1% | 0 |
| PD-L1 | IHC ≥1 to less than 50% | 1 |
| PD-L1 | IHC ≥50% | 2 |
| ARID1A | Cancer-associated mutation present | 1 |
| ARID1A | No mutation present | 0 |
| KEAP1/NFE2L2 | Cancer-associated mutation present | 0 |
| KEAP1/NFE2L2 | No mutation present | 1 |
| STK11/LKB1 | Cancer-associated mutation present | 0 |
| STK11/LKB1 | No mutation present | 1 |
| CDKN2A | Mutation present | 0 |
| CDKN2A | No mutation present | 1 |
| DDR | loss of function in one or more of the following | 1 |
| | genes: ATM, BRCA2, BRIP, MRE11, POLE, | |
| | MSH, PARP1 | |
| DDR | No loss of function in one or more of the | 0 |
| | following genes: ATM, BRCA2, BRIP, | |
| | MRE11, POLE, MSH, PARP1 | |

### I. Individual Biomarker Analysis of Cohort Who Received Atezolizumab Treatment

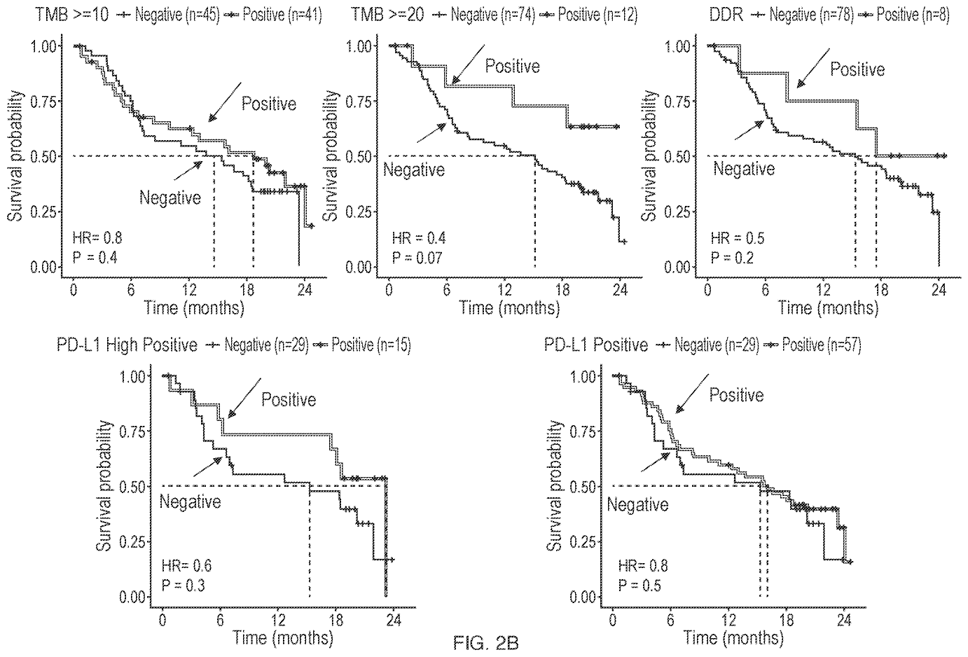

Kaplan-Meier survival curves for 86 non-small cell lung cancer (NSCLC) patients who received atezolizumab treatment were plotted for various individual biomarkers (FIGS. 1A, 2A-B, Table E1). Calculated hazard ratios (HR) indicated that on an individual level, overall survival (OS) was not impacted by the presence or absence of NFE2L2/KEAP1 (HR=1.0) (FIG. 2A). DDR, both TMB levels, ARID1A, and all levels of PD-L1 positive status reported HR ratios below 1, suggesting that the overall survival for those positive for the biomarker was more than the control (negative) group (FIG. 2A-B). STK11/LKB1 and CDKN2A had hazard ratios above 1, indicating that the overall survival for those positive for the biomarker was less than the control (negative) group (FIG. 2A). All calculated HR for individual biomarkers had p values>0.05 and did not reach statistical significance, indicating that the individual biomarker data did not provide sufficient information to conclude the HR confidently for the cohort treated with atezolizumab (FIG. 2A-B).

### II. Composite Biomarker Status Analysis of Cohort Who Received Atezolizumab Treatment

In Example sub-section I, it was shown that individual biomarkers were not sufficient to predict overall survival (i.e., treatment outcome). The identified biomarkers and the accompanying status (e.g. quantity of mutations, the presence of a gene mutation, protein expression levels) was assigned a value and aggregated per individual (n=86) into a composite biomarker score.

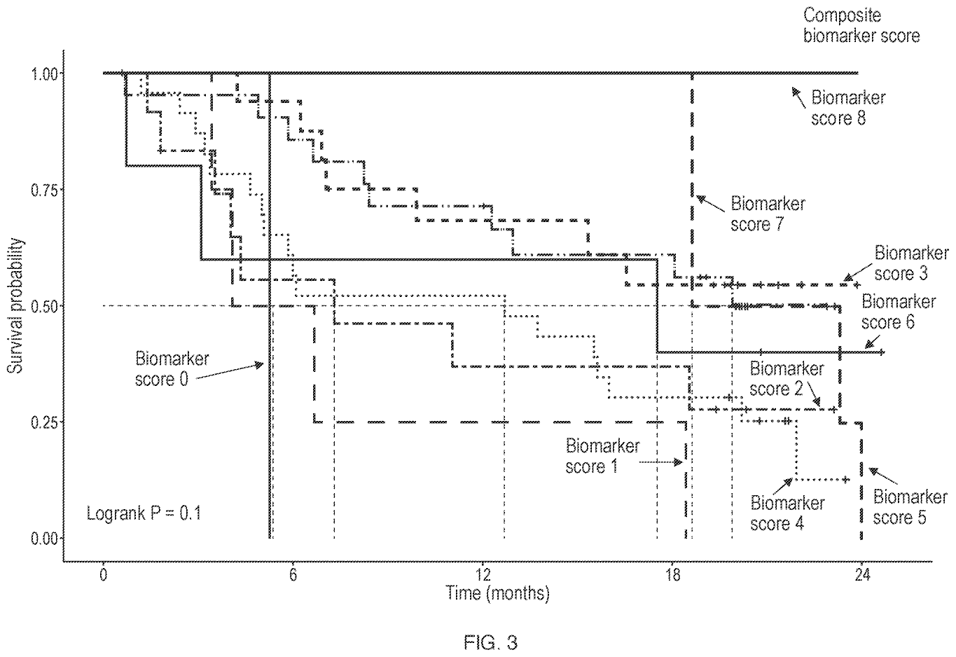

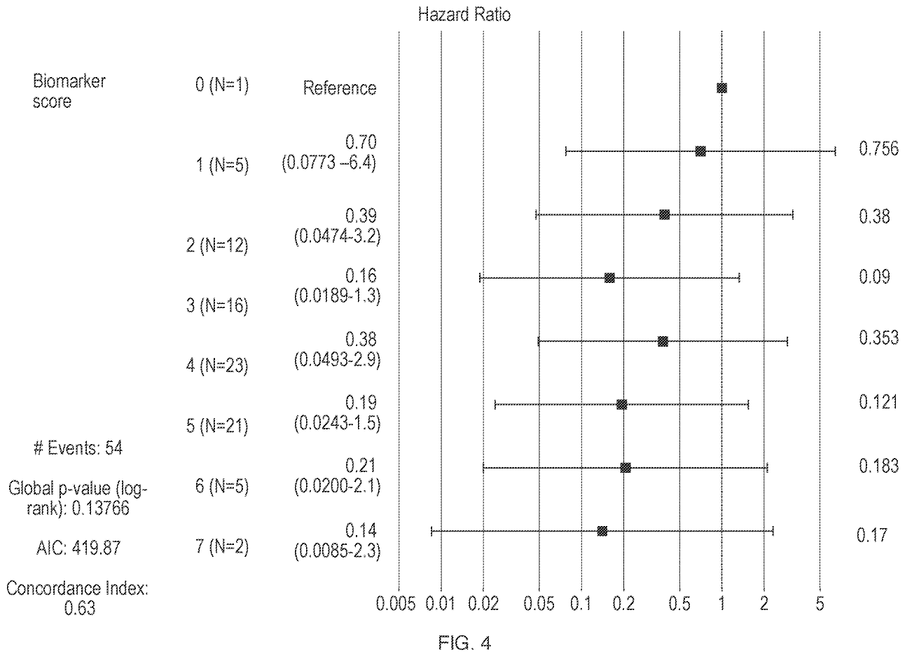

Kaplan-Meier survival curves for 86 non-small cell lung cancer (NSCLC) patients who received atezolizumab treatment were plotted for each composite biomarker score (FIG. 3, Table E1). Calculated hazard ratios (HR) with 95% confidence intervals were reported (FIG. 4). Notably, while all HR values determined for each individual biomarker score was below 1, the 95% confidence interval for all HR reported ranged from HR below 1 to HR above 1 (FIG. 4). Given that calculated HR ratios had p values>0.05 for each biomarker score, this indicated that it could not be determined whether each biomarker score correlated to changes in overall survival in the cohort treated with atezolizumab.

### III. Binned Composite Biomarker Score Analysis of Cohort Who Received Atezolizumab Treatment

In Example sub-section I and II, it was shown that individual and composite biomarker scores were not sufficient to predict overall survival (OS) post treatment. The identified biomarkers and the accompanying status (e.g. quantity of mutations, the presence of a gene mutation, protein expression levels) was assigned a score. These scores were aggregated per individual (n=54) into a composite score (composite biomarker score), then subsequently binned into low, medium and high as described in Table E2.

TABLE E2
Composite biomarker scores are binned into levels to indicate
likelihood of benefitting from the treatment.

| Composite biomarker score | Composite biomarker score level |
|---|---|
| 0-2 | Low |
| 3-4 | Medium |
| 5-8 | High |

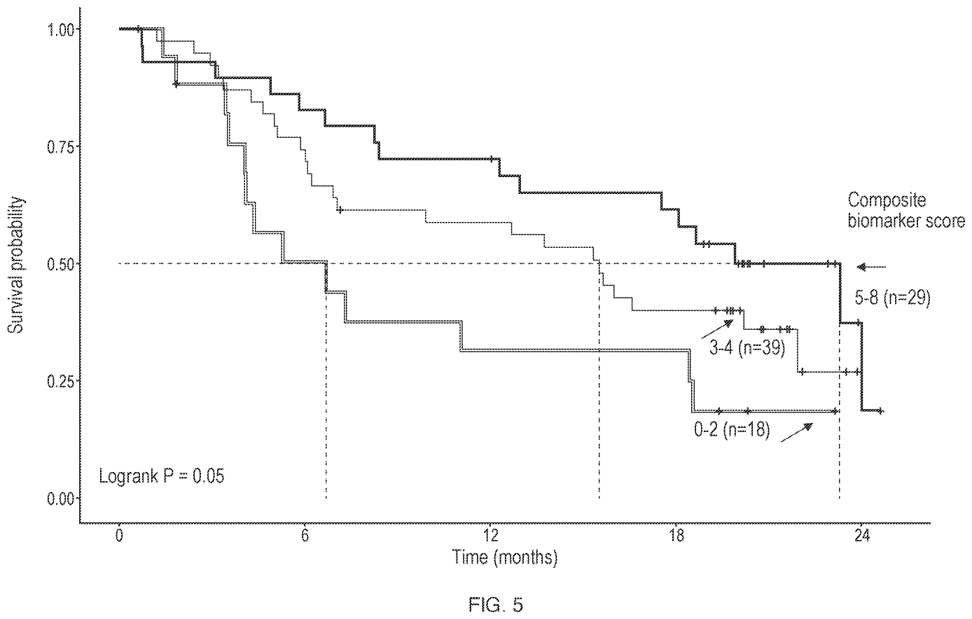

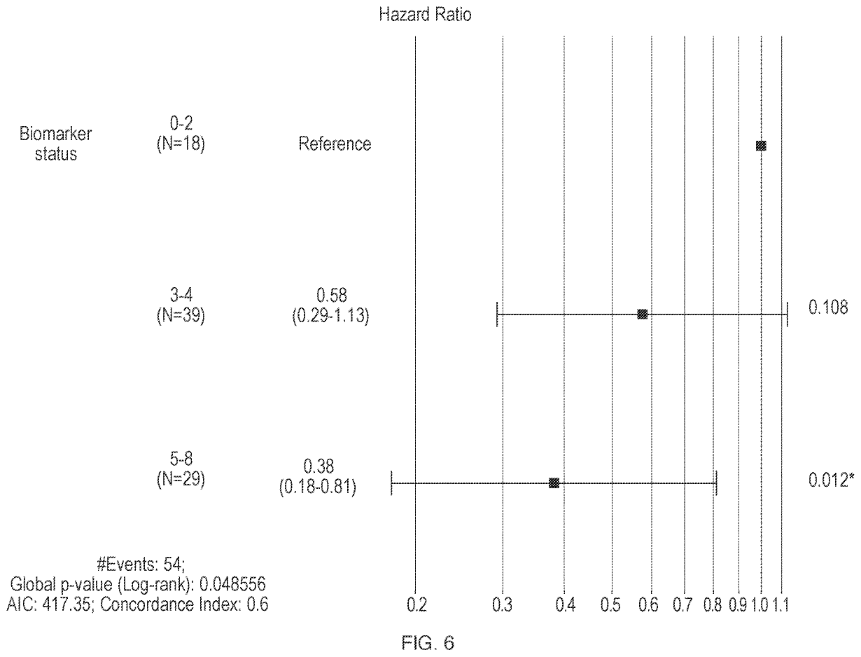

Kaplan-Meier survival curves for 86 non-small cell lung cancer (NSCLC) patients who received atezolizumab treatment were plotted for each binned composite biomarker score (FIG. 5, Table E1). Calculated hazard ratios (HR) with 95% confidence intervals were reported (FIG. 6). High composite biomarker score correlated with improved overall survival within the atezolizumb-treated patients, yielding a median OS of 23 months as compared to 7 months (HR=0.38; 95% CI, 0.18-0.81) between high composite biomarker score versus low composite score (FIG. 5, 6). Notably, for binned composite biomarker scores of 3-4 the reported HR had a p value>0.05, indicating that the observed effect did not reach statistical significance.

The results demonstrate that a high composite biomarker score predicted the efficacy of atezolizumab treatment.

### IV. Binned Composite Biomarker Score Analysis of Cohort Who Received Docetaxel Treatment

In Example sub-section II, it was shown that individual composite biomarker scores were not sufficient to predict overall survival (OS). However, in Example sub-section III, it was shown that binned composite biomarker scores (specifically a composite biomarker score of high) could predict overall survival. The identified biomarkers and the accompanying status (e.g. quantity of mutations, the presence of a gene mutation, protein expression levels) was assigned a score. These scores were aggregated per individual (n=94) into a composite biomarker score, then subsequently binned into levels as described in Table E2.

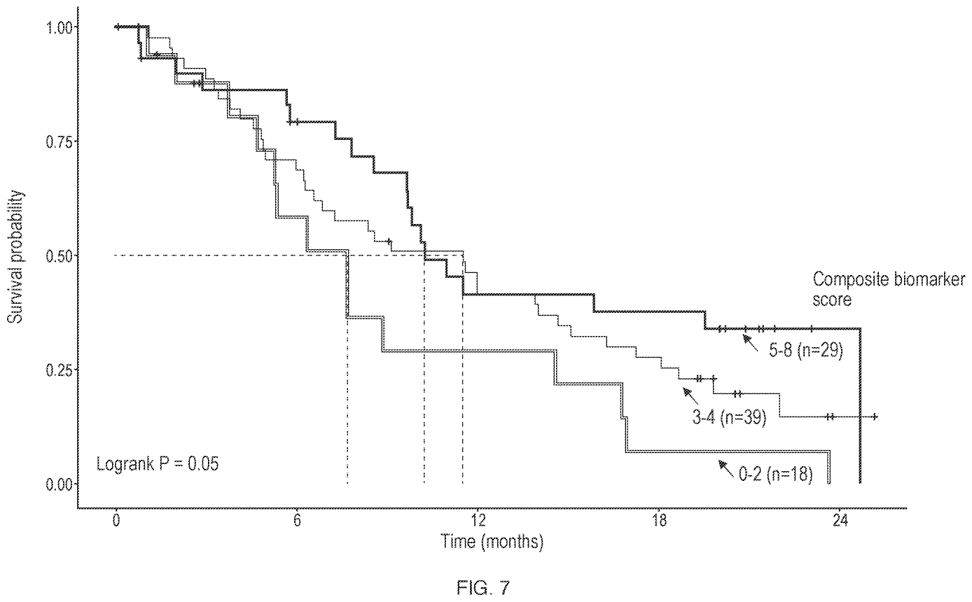

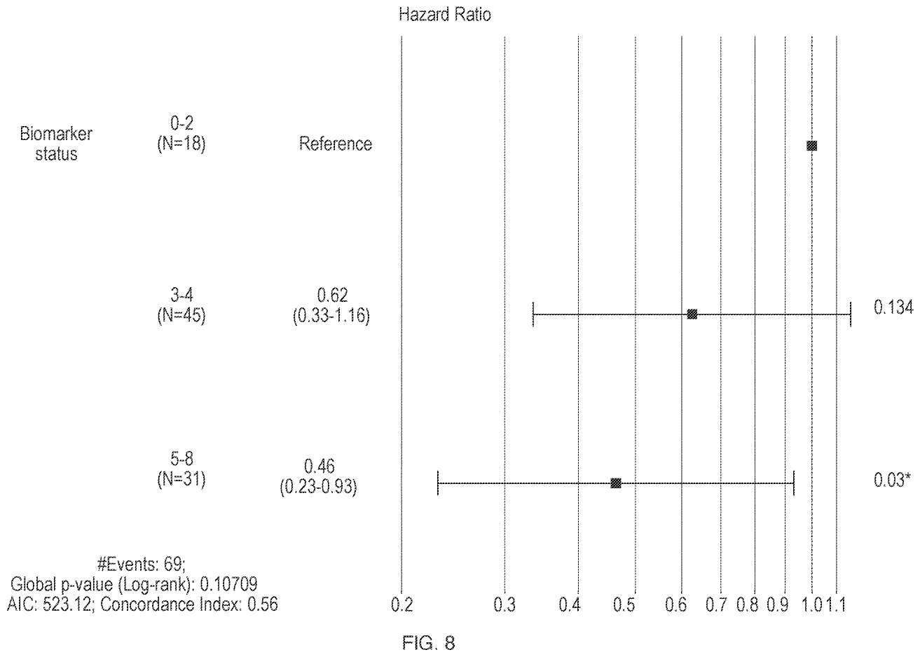

Kaplan-Meier survival curves for 94 non-small cell lung cancer (NSCLC) patients who received docetaxel treatment were plotted for each binned composite biomarker score (FIG. 7, Table E1). Calculated hazard ratios (HR) with 95% confidence intervals were reported (FIG. 8). Notably, for composite biomarker scores of 3-4, the calculated HR value reported a p value>0.05, indicating that the observation that a composite biomarker score of 3-4 led to an improvement overall survival in the cohort treated with docetaxel did not reach statistical significance.

In contrast, for composite biomarker scores of 5-8 the reported HR value was 0.46, and the 95% confidence interval for ranged 0.23-0.93 (FIG. 8). The p value was reported to be 0.03. This indicates that individuals having a composite biomarker score of 5-8 had improved overall survival compared to the control group (composite biomarker score of 0-2). Accordingly, this was supported by the plotted survival median of the binned cohorts receiving docetaxel; those with a composite biomarker score of 5-8 had a survival median at approximately 10 months as compared to 0-2 (approximately 8 months) and 3-4 (approximately 12 months) (FIG. 7).

The results demonstrate that a medium or high composite biomarker score predicted better efficacy of docetaxel treatment.

### V. The Effectiveness of a Binned Composite Biomarker Score Analysis of Azetolizumab Versus Docetaxel Treatment

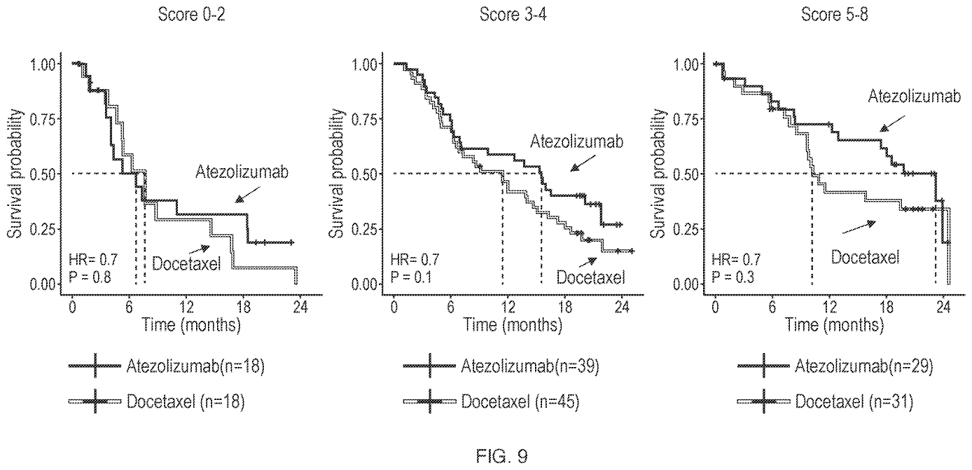

In Example sub-section III and IV, it was shown that binned composite biomarker scores were effective indicators to predict an increase in overall survival over the control group. Composite biomarker scores were calculated for the cohorts that received either Azetolizumab and docetaxel treatments, then binned into composite biomarker scores to identify levels. Kaplan-Meier curves were then calculated and analyzed to see which treatment was more effective in the selected patient population (FIGS. 1A, 1B, 9). High composite biomarker scores correlated with a median OS of 23 months vs. 10 months between atezolizumab and docetaxel treated patients within the high composite biomarker score subpopulations (HR=0.68; 95% CI, 0.34-1.3).

Taken together, these results demonstrate that the composite biomarker score is more accurate and informative than any single assessed biomarker in guiding immune checkpoint inhibitor (ICI) treatment selection in NSCLC patients.

### M. Example 2: Composite Biomarker Scores as Predictors of Survival Outcome

This Example shows that integrating single biomarkers into a binned, composite biomarker is useful in identifying NSCLC patients that have better overall survival outcomes. This Example also provides another method of determining composite biomarker scores.

### Methods

19,069 patients with advanced stage non-cell lung carcinoma (NSCLC) who had over two visits within a defined network on or after Jan. 1, 2021.

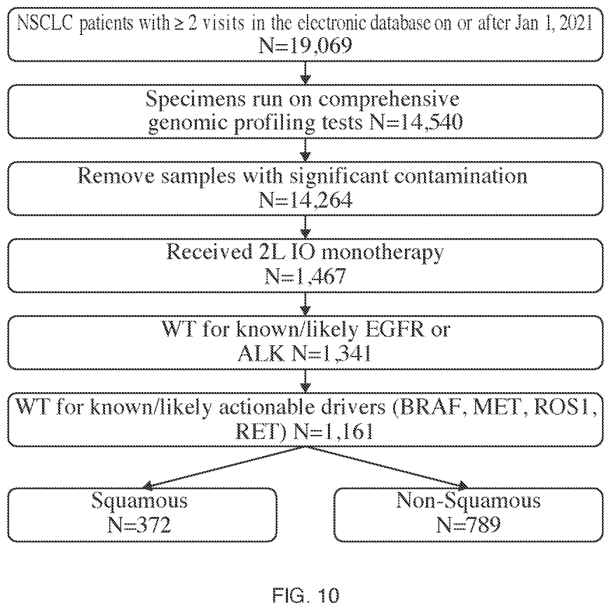

Of these patients, 14,540 participants further selected based on whether they were profiled using a tissue based comprehensive genomic profiling test and were selected for further analysis. Samples were screened for significant contamination, then individuals who received second line monotherapy were identified (N=1,467). Those who were known or likely EGFR or ALK wild-type were selected. These individuals were then further refined to be wild-type for known or likely known actionable drivers (BRAF, MET, ROS1, RET) (n=1,161). The remaining individuals were then sorted into squamous (N=372) or non-squamous (N=789) (FIG. 10).

7 individual biomarkers were identified and the hazard ratios were determined for individuals who were either positive or negative for the biomarker (Table E3). A nominal system was subsequently identified, wherein composite scores were based on individual biomarker positivity and further categorized into levels (composite biomarker score of −1 or lower [low]; biomarker score of 0-2 [medium]; biomarker score of 3 or higher [high]). A Cox proportional hazards regression model was then used to investigate binning composite biomarker scores on determining overall survival of NSCLC patients.

TABLE E3
Components included in the composite biomarker score

| Biomarker | Status | Score |
|---|---|---|
| TMB (tumor | <10 mut/Mb | 0 |
| mutational | | |
| burden) | | |
| TMB (tumor | =10 to less than 20 mut/Mb | 1 |
| mutational | | |
| burden) | | |
| TMB (tumor | ≥20 mut/Mb | 2 |
| mutational | | |
| burden) | | |
| PD-L1 | High positive; score of IC3 or TC3 by a | 2 |
| | monoclonal anti-PD-L1 qualitative | |
| | immunohistochemical assay | |
| PD-L1 | Low positive; score of TC0/1/2, TC1/2/3, TC2, | 1 |
| | or TC2/3 and IC0, IC1, or IC2 by a monoclonal | |
| | anti-PD-L1 qualitative immunohistochemical | |
| | assay | |
| ARID1A | Cancer-associated mutation present or likely | 1 |
| | present | |
| ARID1A | No mutation present | 0 |
| KEAP1/ | Cancer-associated mutation present | −1 |
| NFE2L2 | | |
| KEAP1/ | No mutation present | 0 |
| NFE2L2 | | |
| STK11/LKB1 | Cancer-associated mutation present or likely | −1 |
| | present | |
| STK11/LKB1 | No mutation present | 0 |
| CDKN2A | Mutation present (homozygous deletion) | −1 |
| DDR | loss of function in one or more of the following | 1 |
| | genes: MLH1, MSH2, MSH6, PMS2, ATM, | |
| | BRCA1, BRCA2, PALB2, BRIP1, BARD1 | |
| DDR | No loss of function in one or more of the | 0 |
| | following genes: MLH1, MSH2, MSH6, PMS2, | |
| | ATM, BRCA1, BRCA2, PALB2, BRIP1, | |
| | BARD1 | |

### I. Composite Biomarker Status Analysis of the NSCLC Cohort

In Example I sub-section I, it was shown that individual biomarkers were not sufficient to predict overall survival (i.e., treatment outcome). Thus, the identified biomarkers and the accompanying status (e.g. quantity of mutations, the presence of a gene mutation, protein expression levels) was assigned a value and aggregated per individual (n=1,161) into a composite biomarker score.

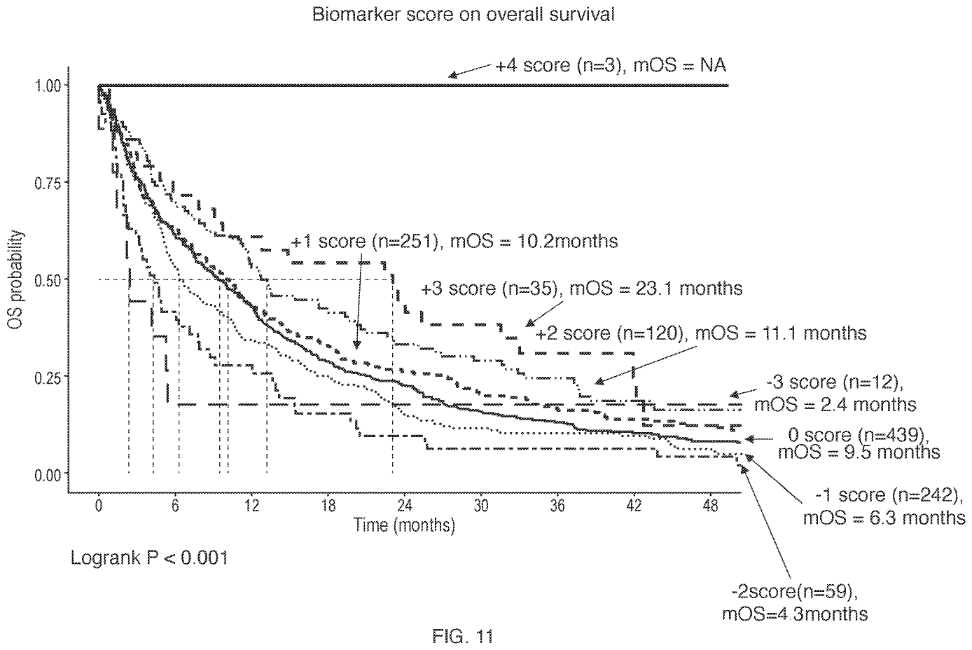

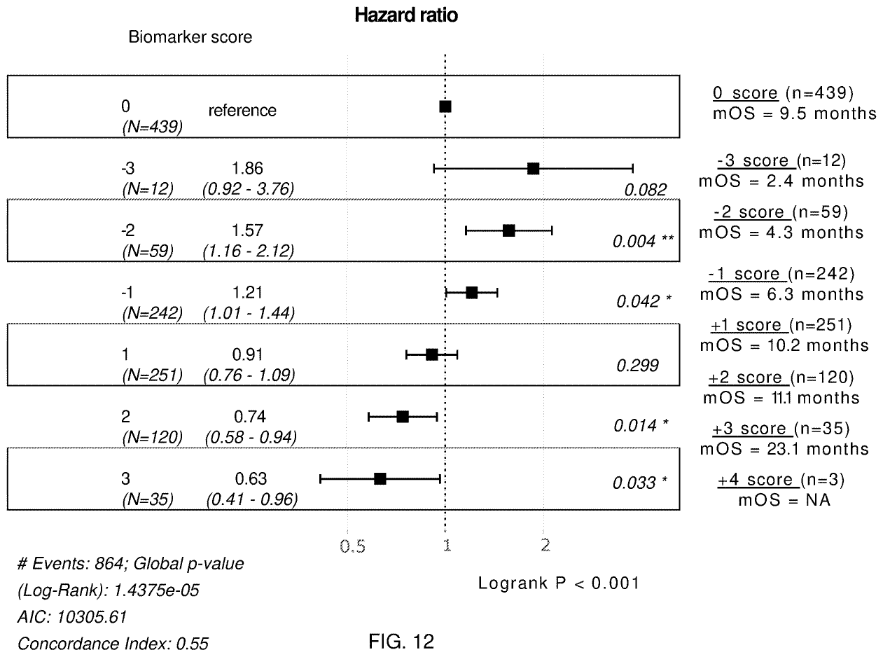

Kaplan-Meier survival curves for non-small cell lung cancer (NSCLC) patients plotted for each composite biomarker score (FIG. 11, Table E3). Calculated hazard ratios (HR) with 95% confidence intervals were reported (FIG. 12). Notably, all positive composite biomarker scores had HR values was below 1, and all negative composite biomarker scores had HR values above 1. The 95% confidence interval for all HR reported ranged from HR below 1 to HR above 1 except for the composite biomarker scores of 2 and 3. (FIG. 11, FIG. 12). However, the calculated HR ratios had p values>0.05 for each composite biomarker scores of −3 and 1, this indicated that that only some of the composite biomarker scores correlated to the changes in overall survival in patients who had received second line immunotherapy (IO) with an immune checkpoint inhibitor (monotherapy).

### II. Binned Composite Biomarker Score Analysis of NSCLC Patients

In Example 2, sub-section I, it was shown that individual composite biomarker scores were not sufficient to predict overall survival (OS). Thus, the identified biomarkers and the accompanying status (e.g. quantity of mutations, the presence of a gene mutation, protein expression levels) was assigned a score (Table E3), aggregated per individual (n=1,161) into a composite score (composite biomarker score), then binned into low, medium and high as described in Table E4.

TABLE E4
Composite biomarker scores are binned into levels

| Composite biomarker score | Composite biomarker score level |
|---|---|
| −1 and below | Low |
| 0-2 | Medium |
| +3 and above | High |

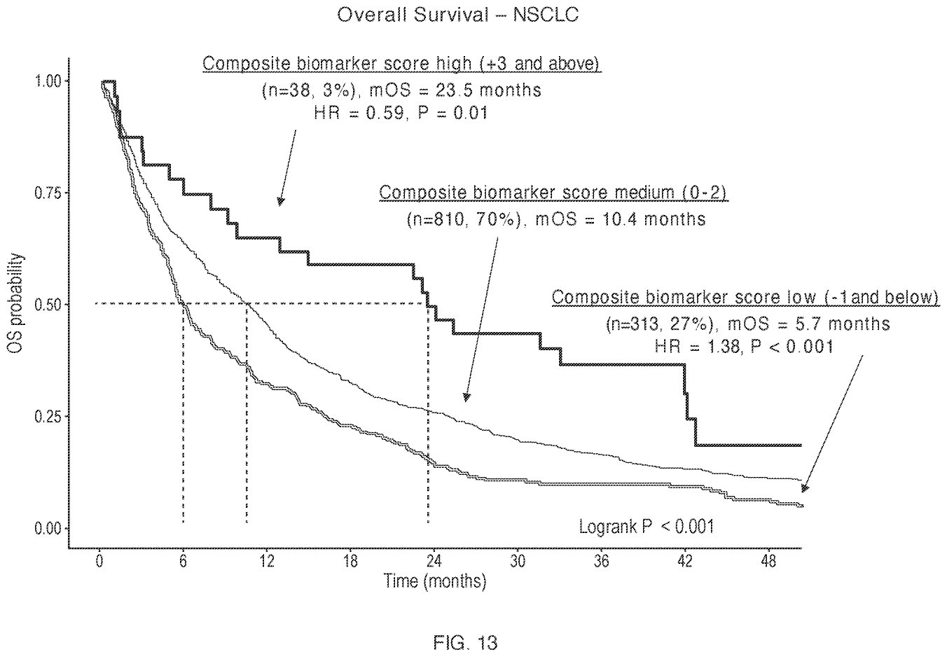

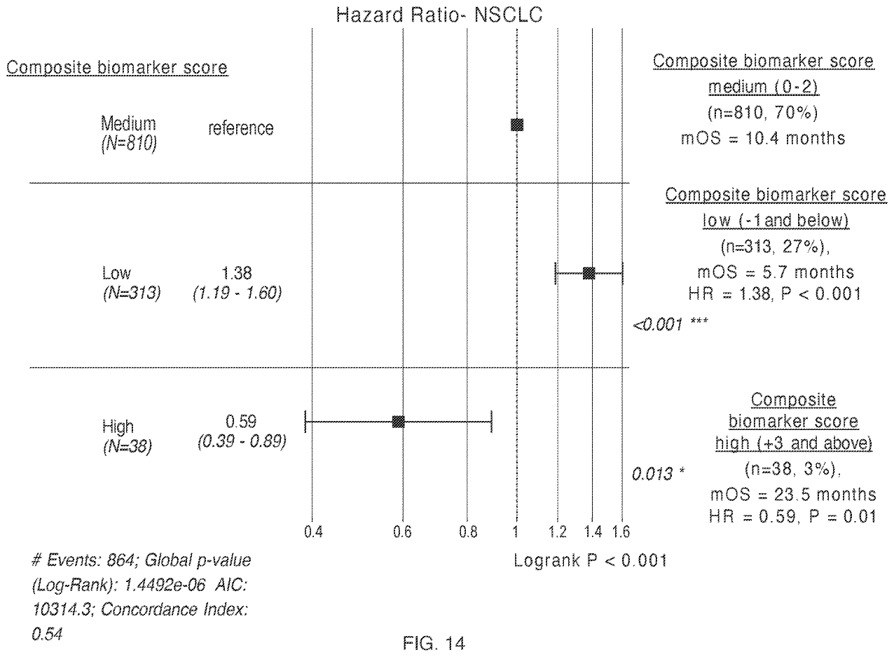

Kaplan-Meier survival curves for 1,161 non-small cell lung cancer (NSCLC) patients were plotted for each binned composite biomarker score (FIG. 13, Table E3, Table E4). Calculated hazard ratios (HR) with 95% confidence intervals were reported (FIG. 14). Low composite biomarker score correlated with lower overall survival for NSCLC patients who had received second line immunotherapy (IO) with an immune checkpoint inhibitor (monotherapy), yielding a median OS of 5.7 months as compared to 10.4 months (HR=1.38; 95% CI, 1.19-1.60) between low composite biomarker score versus medium composite score (FIG. 13, FIG. 14). Notably, for binned composite biomarker scores of −1 and lower, the reported HR had a p value<0.001, indicating that the observed effect reached statistical significance. Similarly, high composite biomarker score correlated with overall longer overall survival versus the medium composite score for NSCLC patients, yielding a median OS of 23.5 months compared to 10.4 months (HR=0.59; 95% CI, 0.39-0.89) (FIG. 13, FIG. 14). The p value was reported to be 0.01, indicating that this observation was statistically significant.

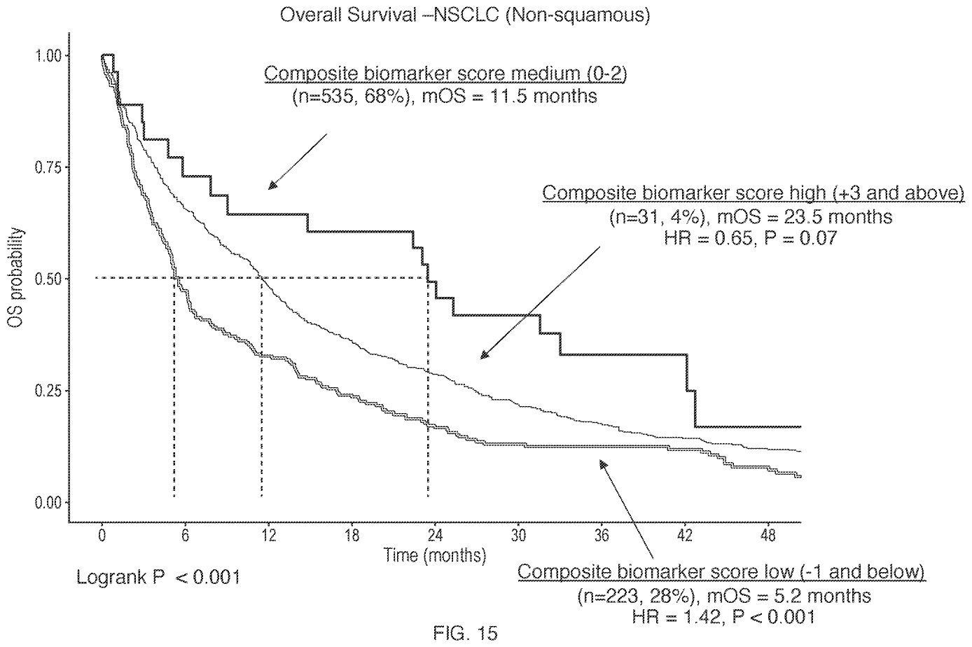

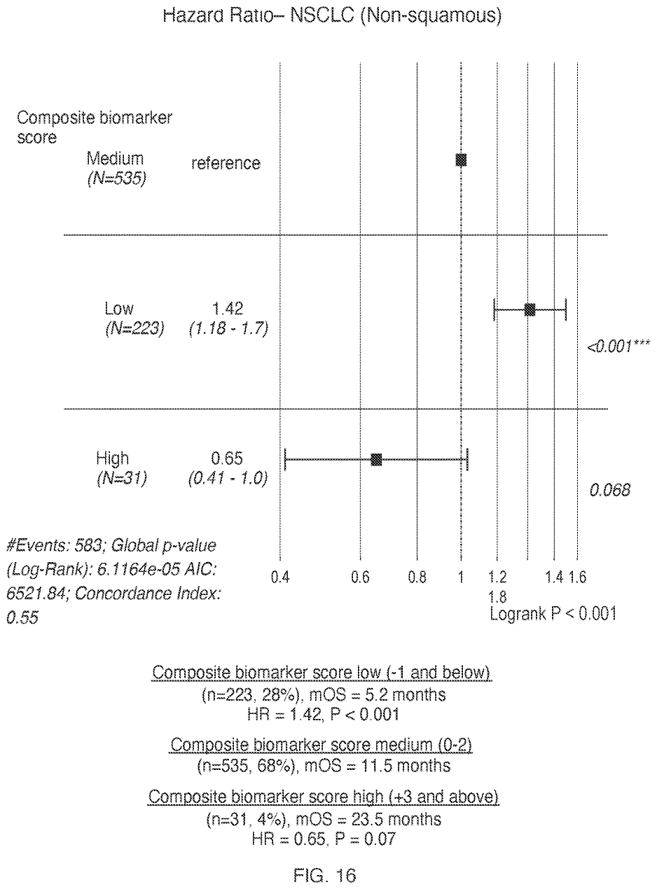

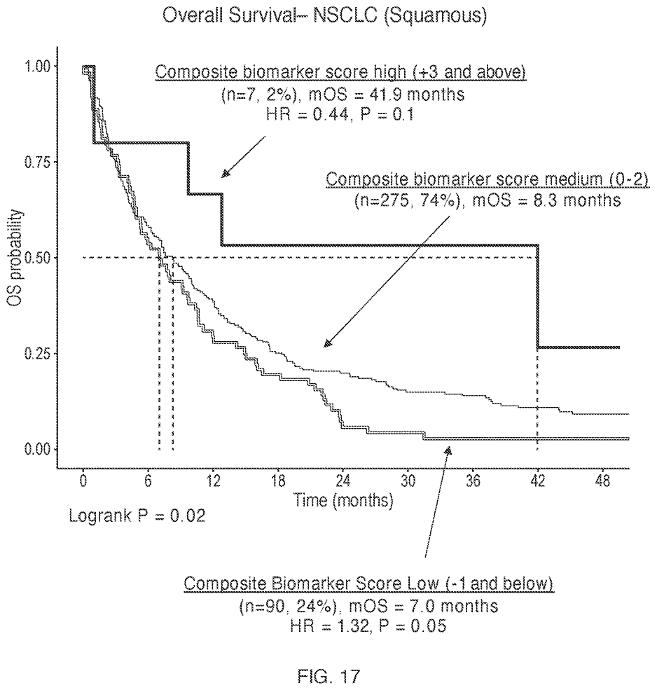

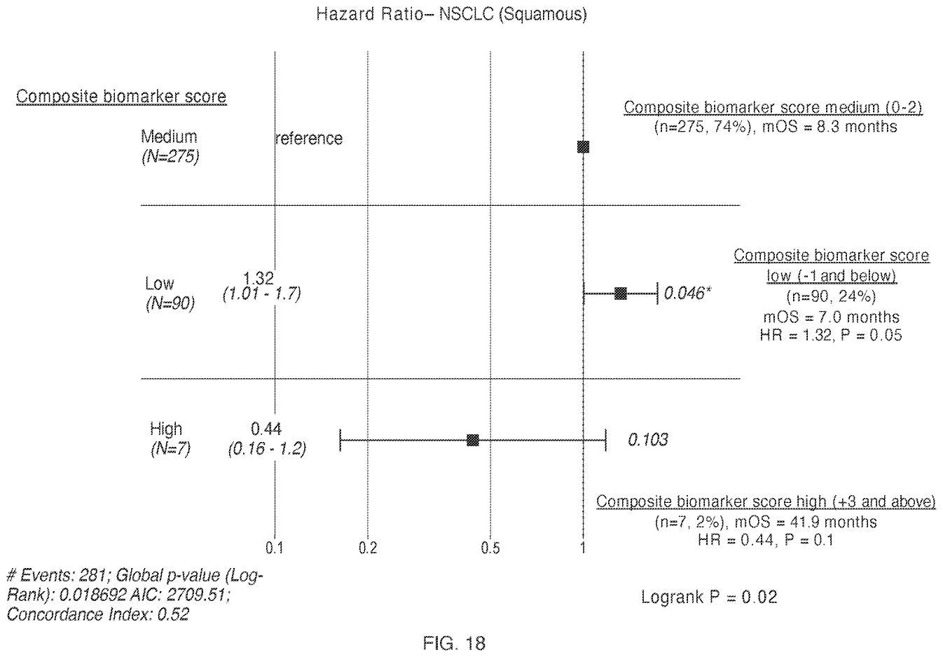

To identify whether further segregating the analyzed groups would refine the correlation of overall survival with binned composite biomarker score scores, Kaplan-Meier curves were plotted and hazard ratios were calculated for non-squamous (FIG. 15, FIG. 16) and squamous patients (FIG. 17, FIG. 18). In both analyses, composite biomarker scores of low (−1 or lower) demonstrated a statistically significant reduction in overall survival compared to a composite biomarker score of medium (composite biomarker score of 0-2) (FIG. 15, FIG. 16, FIG. 17, FIG. 18). Similarly, binned composite biomarker scores of high (3 or higher) had an observable, but statistically insignificant increase in overall survival compared to a binned biomarker score of medium (0-2).

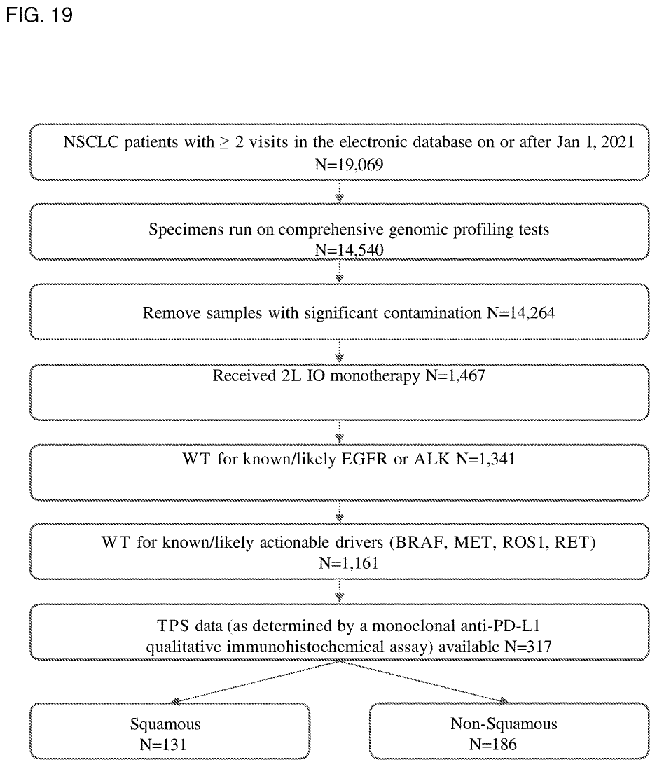

For the composite biomarker analysis, PD-L1 status was determined by looking at the combined positive score (CPS) as expressed as TC, or looking at the proportion of tumor area occupied by PD-L1 expressing tumor-infiltrating immune cells (IC) (Table 3). The CPS reports the number of PD-L1 positive cells (both tumor and non-tumor) divided by the total number of viable tumor cells, multiplied by 100. To determine if restricting the analysis to include patients with PD-L1 tumor proportion score (TPS) would yield a better correlation to overall survival in NSCLC patients who had received second line immunotherapy (IO) with an immune checkpoint inhibitor (monotherapy), the data were further filtered to select for those who had PD-L1 TPS data available (n=317) (FIG. 19). TPS reports the number of PD-L1 positive cells divided by total number of all tumor cells, multiplied by 100.

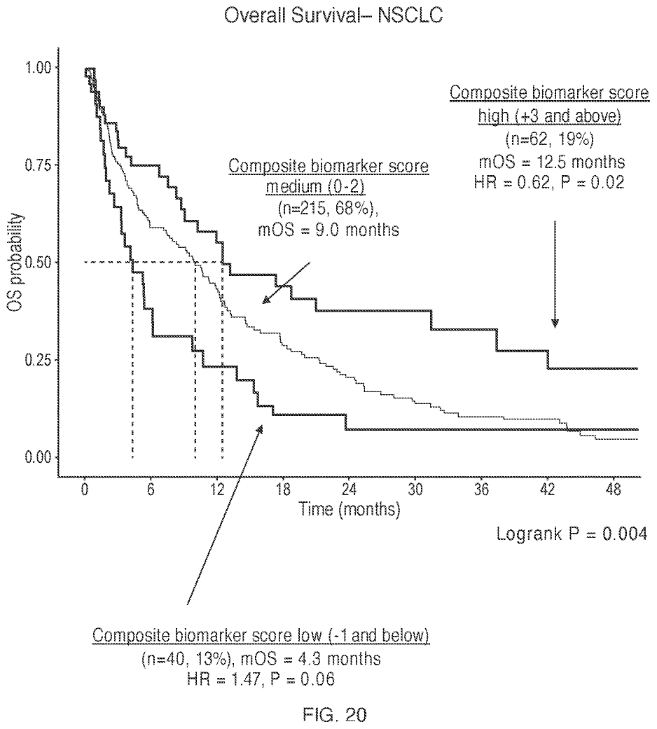

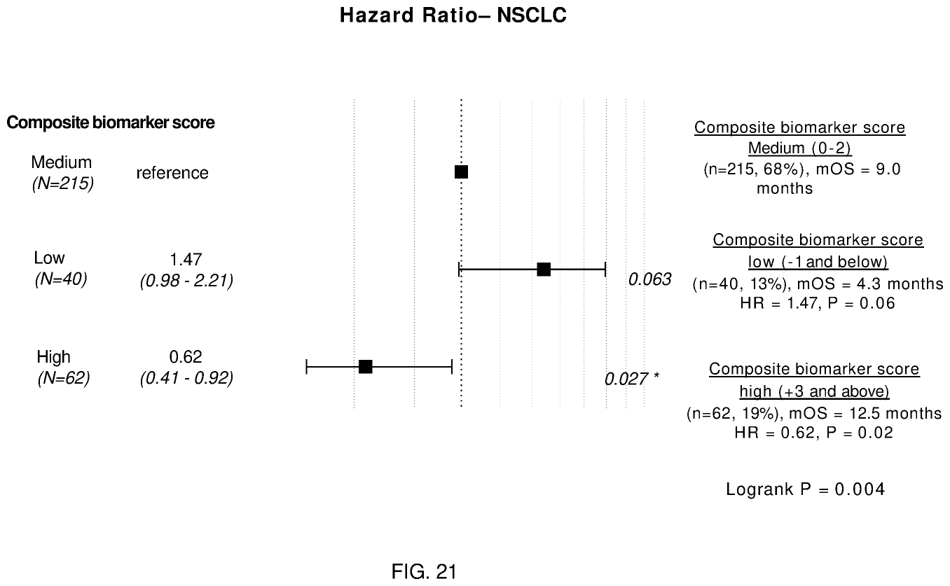

Kaplan-Meier survival curves for the remaining 317 non-small cell lung cancer (NSCLC) patients were plotted for each binned composite biomarker score (FIG. 20, Table E3, Table E4). Calculated hazard ratios (HR) with 95% confidence intervals were reported (FIG. 21). Low composite biomarker score correlated with lower overall survival for NSCLC patients, yielding a median OS of 4.3 months as compared to 9 months (HR=1.47; 95% CI, 0.98-2.21) between low composite biomarker score versus medium composite score (FIG. 20, FIG. 21). Notably, for binned composite biomarker scores of −1 and lower, the reported HR had a p value<0.06, indicating that the observed effect did not reach statistical significance. A high composite biomarker score correlated with overall longer overall survival versus the medium composite score for NSCLC patients, yielding a median OS of 12.5 months compared to 9 months (HR=0.62; 95% CI, 0.41-0.92) (FIG. 20, FIG. 21). The p value was reported to be 0.02, indicating that this observation was statistically significant.

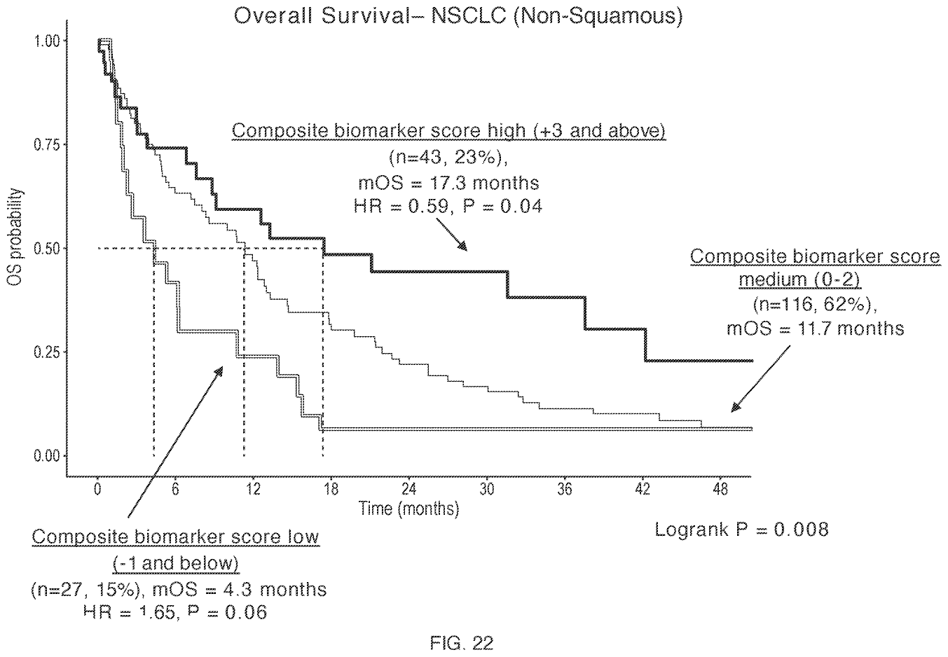

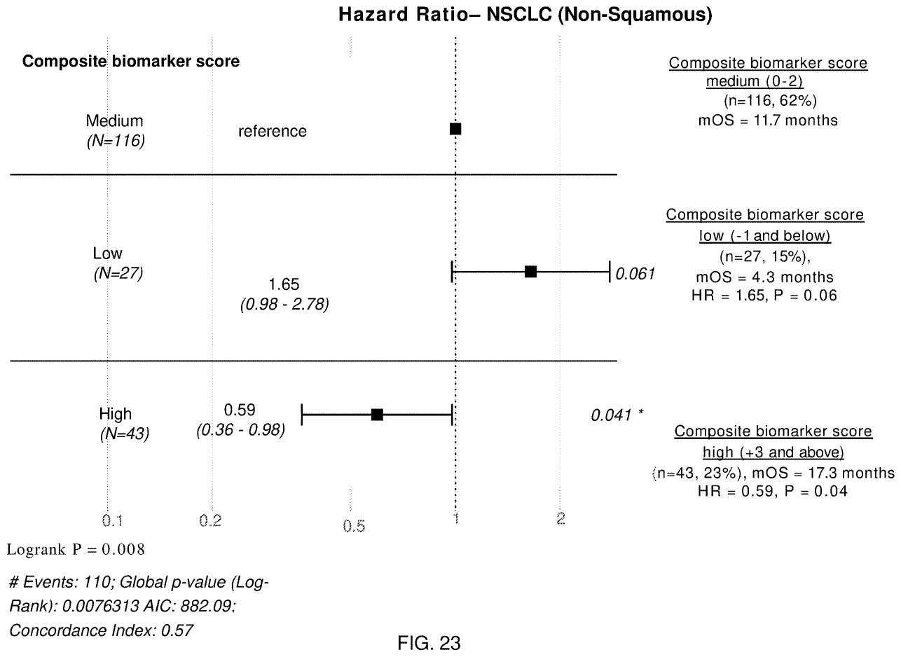

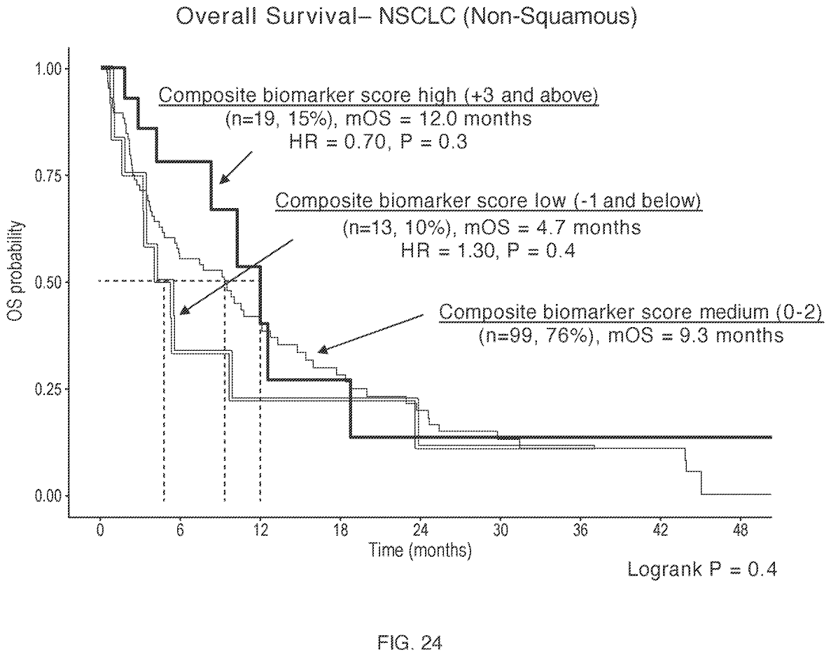

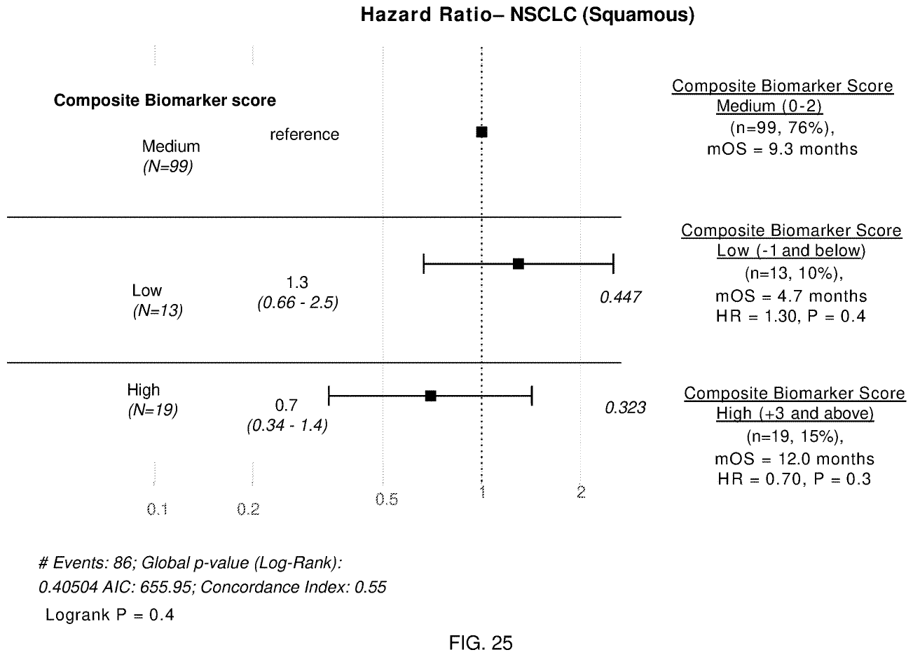

To identify whether further segregating the analyzed groups would refine the correlation of overall survival with the binned composite scores, Kaplan-Meier curves were plotted and hazard ratios were calculated for non-squamous (FIG. 22, FIG. 23) and squamous patients (FIG. 24, FIG. 25). In both analyses, composite biomarker scores of low (−1 or lower) correlated with a reduction in overall survival compared to a composite biomarker score of medium, but the results were not statistically significant (FIG. 22, FIG. 23, FIG. 24, FIG. 25). Similarly, a binned composite biomarker of high (3 or higher) had an observable increase in overall survival compared to a binned composite biomarker score of medium (0-2), but only the squamous population reached statistical significance (p=0.04; 17.3 months overall survival compared to 11.7 months).

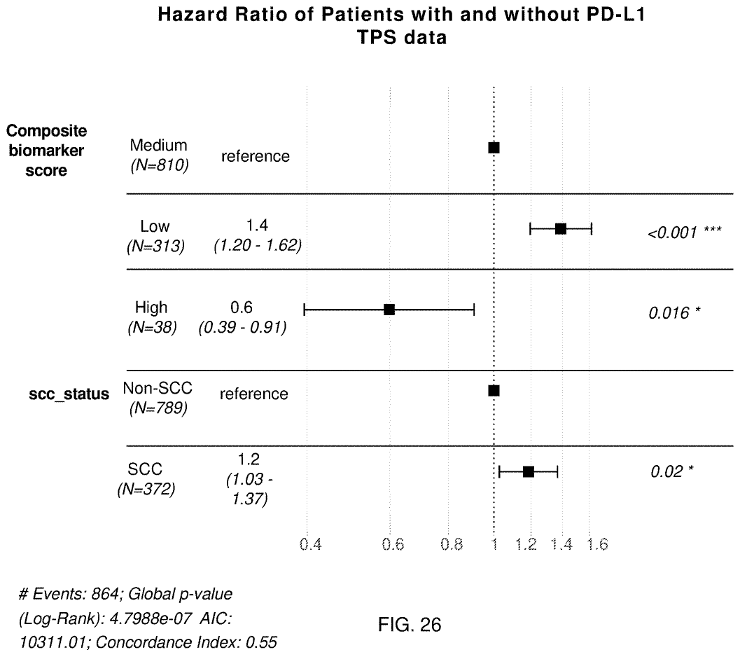

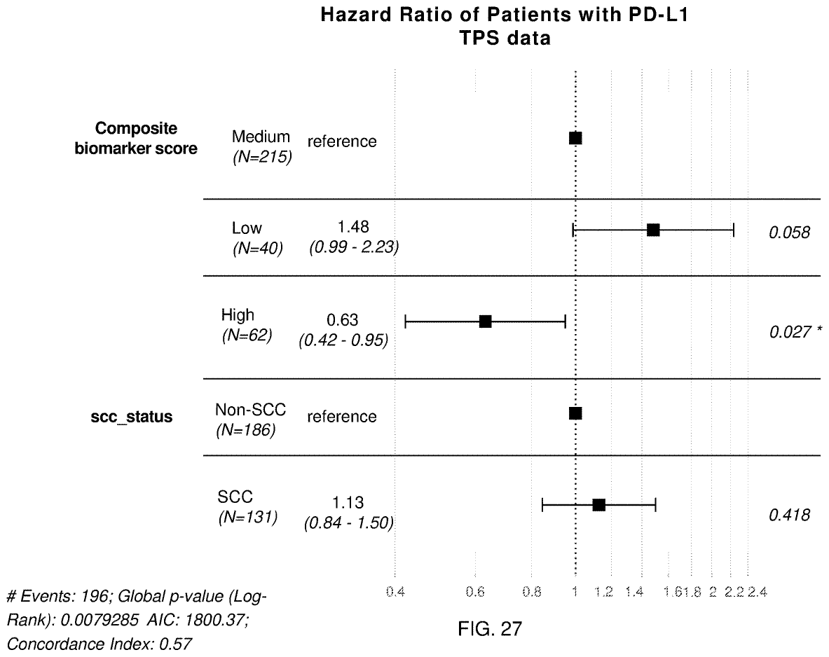

Thus, the hazard ratios of patients both with and without PD-L1 TPS data was able to be used to determine statistically significant correlations in overall survival for all binned composite biomarker scores (FIG. 26), whereas the hazard ratios of patients with PD-L1 TPS data did not reach statistical significance except for one (composite biomarker score of high) (FIG. 27).

Squamous NSCLC is known to be associated with a poorer prognosis. Overall survival based on squamous versus non-squamous status was plotted to determine of the population analyzed was reflective of real-world observations. Within the cohort of patients with and without PD-L1 TPS data available, squamous individuals had a statistically significant increase in hazard ratio (e.g., correlating with a poorer OS survival) compared to the non-squamous population (FIG. 26).

Sorting by squamous versus non-squamous status within the patient population where TPS data was available did not significantly influence overall survival, validating that further restricting the patient population by PD-L1 TPS data availability was not necessary (FIG. 27).

The results demonstrate that in NSCLC patients who had received second line immunotherapy (IO) with an immune checkpoint inhibitor (monotherapy) (and irrelevant of squamous versus non-squamous status), a low composite biomarker score was predictive of a lower overall survival, and a high composite biomarker score was predictive of a higher overall survival. This also indicates that binned composite biomarker scores can be useful in determining outcomes that would not otherwise be apparent. Taken together, these results also demonstrate that the composite biomarker score is more accurate and informative than any single assessed biomarker in determining OS in NSCLC patients.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### EXEMPLARY EMBODIMENTS

The following embodiments are exemplary and not intended to limit the scope of the invention described herein.

Embodiment 1. A method of identifying a subject with a cancer who will likely benefit from a treatment comprising an immune checkpoint inhibitor, the method comprising determining a composite biomarker score; wherein the composite biomarker score is determined based on at least three of the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Embodiment 2. A method of identifying a subject with a cancer who will likely benefit from a treatment comprising an immune checkpoint inhibitor, the method comprising determining a composite biomarker score; wherein the composite biomarker score is determined based on the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e)the presence of a cancer-associated mutation in a STK11/LKB1 gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Embodiment 3. A method of predicting if a subject with a cancer will likely benefit from a treatment comprising an immune checkpoint inhibitor, the method comprising determining a composite biomarker score; wherein the composite biomarker score is determined based on at least three of the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 pathway gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Embodiment 4. A method of predicting if a subject with a cancer will likely benefit from a treatment comprising an immune checkpoint inhibitor, the method comprising determining a composite biomarker score; wherein the composite biomarker score is determined based on the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 pathway gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Embodiment 5. A method of stratifying a subject with a cancer for treatment comprising an immune checkpoint inhibitor, the method comprising determining a composite biomarker score; wherein the composite biomarker score is determined based on at least three of the following (a)-(g): (a) the tumor mutational burden (TMB) score of a tumor sample associated with the cancer, (b) the percent of cells in the tumor sample that are positive for PD-L1, (c) the presence of a cancer-associated mutation in a ARID1A gene in the sample, (d) the presence of a cancer-associated mutation in a KEAP1/NFE2L2 pathway gene in the sample, (e) the presence of a cancer-associated mutation in a STK11/LKB1 pathway gene in the sample, (f) the presence of a cancer-associated mutation in a CDKN2A gene in the sample, and (g) the presence of a cancer-associated mutation in a DNA damage response (DDR) gene in the sample; and determining, based on the determined composite biomarker score, whether the subject will likely benefit from the treatment comprising the immune checkpoint inhibitor.

Embodiment 6. The method of any one of Embodiments 1-5, wherein the composite biomarker score is further based on (h): (h) one or more human leukocyte antigen (HLA) Class I genes of the cancer are determined to exhibit loss of heterozygosity (LOH).

Embodiment 7. The method of Embodiment 6, wherein the one or more HLA Class I genes are selected from the group consisting of HLA-A, HLA-B, HLA-C, HLA-DP, HLA-DQ, and HLA-DR.

Embodiment 8. The method of Embodiment 6 or Embodiment 7, wherein the one or more HLA Class I genes comprise at least HLA-A, HLA-B, and HLA-C.

Embodiment 9. The method of any one of Embodiments 1-5, further comprising characterizing the subject as: (i) having a low likelihood of benefitting from the treatment comprising the immune checkpoint inhibitor, (ii) having an indeterminate or medium likelihood of benefitting from the treatment comprising the immune checkpoint inhibitor, or (iii) having a high likelihood of benefitting from the treatment comprising the immune checkpoint inhibitor; wherein the subject is characterized as one of (i)-(iii) based on the determined composite biomarker score.

Embodiment 10. The method of any one of Embodiments 1-9, wherein the composite biomarker score is calculated by adding to the score for each of (a)-(g), or (a)-(h), that are met.

Embodiment 11. The method of Embodiment 10, wherein each of (a)-(g) or (a)-(h) that are met increments the score by at least 1.

Embodiment 12. The method of Embodiment 10 or Embodiment 11, wherein the composite biomarker score starts at 0 and is incremented for each of (a)-(g) or each of (a)-(h) that are met.

Embodiment 13. The method of Embodiment 10, wherein each of (a)-(c) and (g) that are met increments the score by at least 1, and wherein each of (d)-(f) that are met decreases the score by 1.

Embodiment 14. The method of Embodiment 10 or Embodiment 13, wherein the composite biomarker score starts at 0 and is incremented for each of (a)-(c) and (g) that are met; and is decreased by 1 for each of (d)-(f) that are met.

Embodiment 15. The method of any one of Embodiments 10-14, wherein the determination of whether the subject will likely benefit from the treatment comprising the immune checkpoint inhibitor is based on the composite biomarker score being at least a threshold value.

Embodiment 16. The method of any one of Embodiments 1-15, wherein the composite biomarker score is incremented if the TMB score of (a) is at least 10.

Embodiment 17. The method of Embodiment 16, wherein if (a) is met, the composite biomarker score is incremented by one.

Embodiment 18. The method of any one of Embodiments 1-15, wherein the composite biomarker score is incremented if the TMB score of (a) is at least 20.

Embodiment 19. The method of Embodiment 18, wherein if (a) is met, the composite biomarker score is incremented by two.

Embodiment 20. The method of any one of Embodiments 1-19, wherein the composite biomarker score is incremented based on the TMB score of (a) as follows: (i) if the TMB score is less than 10, the composite biomarker score is not incremented, (ii) if the TMB score is at least 10, the composite biomarker score is incremented, and (iii) if the TMB score is at least 20, the composite biomarker score is further incremented over the increment of (ii).

Embodiment 21. The method of Embodiment 18, wherein the composite biomarker score is incremented by 1 if (ii) is met and incremented by 2 if both (ii) and (iii) are met.

Embodiment 22. The method of any one of Embodiments 1-21, wherein (b) is based on the percent of cells that are positive for PD-L1 being at least 1%.

Embodiment 23. The method of Embodiments 22, wherein if (b) is met, the composite biomarker score is incremented by one.

Embodiment 24. The method of any one of Embodiments 1-23, wherein (b) is based on the percent of cells that are positive for PD-L1 being at least 50%.

Embodiment 25. The method of Embodiment 24, wherein if (b) is met, the composite biomarker score is incremented by two.

Embodiment 26. The method of any one of Embodiments 1-25, wherein the composite biomarker score is incremented based on the percent of cells in the sample that are positive for PD-L1 of (b) as follows: (i) if the percent of cells in the sample that are positive for PD-L1 is less than 1%, the composite biomarker score is not incremented, (ii) if the percent of cells in the sample that are positive for PD-L1 is at least 1% and less than 50%, the composite biomarker score is incremented, and (iii) if the percent of cells in the sample that are positive for PD-L1 is at least 50%, the composite biomarker score is further incremented over the increment of (ii).

Embodiment 27. The method of Embodiment 26, wherein the composite biomarker score is incremented by 1 if (ii) is met and incremented by 2 if both (ii) and (iii) are met.

Embodiment 28. The method of any one of Embodiments 12-27, wherein the threshold value is 2.

Embodiment 29. The method of any one of Embodiments 12-27, wherein the threshold value is 3.

Embodiment 30. The method of any one of Embodiments 12-27, wherein the threshold value is 4.

Embodiment 31. The method of any one of Embodiments 12-27, wherein the threshold value is 5.

Embodiment 32. The method of any one of Embodiments 12-27, wherein the threshold value is 6.

Embodiment 33. The method of any one of Embodiments 12-27, wherein the threshold value is 7.

Embodiment 34. The method of any one of Embodiments 13-27, wherein the threshold value is −3.

Embodiment 35. The method of any one of Embodiments 13-27, wherein the threshold value is −2.

Embodiment 36. The method of any one of Embodiments 13-27, wherein the threshold value is −1.

Embodiment 37. The method of any one of Embodiments 13-27, wherein the threshold value is 0.

Embodiment 38. The method of any one of Embodiments 13-27, wherein the threshold value is 1.

Embodiment 39. The method of any one of Embodiments 12-38, wherein if the composite biomarker score is less than the threshold value, the subject is identified as one who will not benefit from treatment with the immune checkpoint inhibitor.

Embodiment 40. The method of any one of embodiments 12-38, wherein if the composite biomarker score is less than the threshold value, the subject is identified as one who will not benefit from a monotherapy treatment with the immune checkpoint inhibitor.

Embodiment 41. The method of any one of Embodiments 1-40, wherein the TMB score is determined based on between about 100 kb to about 10 Mb of genomic sequence.

Embodiment 42. The method of any one of Embodiments 1-41, wherein the TMB score is determined based on between about 0.8 Mb to about 1.1 Mb of genomic sequence.

Embodiment 43. The method of any one of Embodiments 1-42, wherein the DDR genes comprise one or more of ATM, BRCA2, BRIP1, MRE11, POLE, MSH2, PARP1, MLH1, MSH6, PMS2, BRCA1, PALB2, and BARD1.

Embodiment 44. The method of any one of Embodiments 1-43, wherein (g) is based on a cancer-associated mutation reducing gene expression of one or more genes selected from the list consisting of ATM, BRCA2, BRIP1, MRE11, POLE, MSH2, PARP1, MLH1, MSH6, PMS2, BRCA1, PALB2, and BARD1.

Embodiment 45. The method of any one of Embodiments 1-44, wherein the DDR genes comprise one or more of ATM, BRCA2, BRIP1, MRE11, POLE, MSH2, and PARP1.

Embodiment 46. The method of any one of Embodiments 1-44, wherein the DDR genes comprise one or more of MLH1, MSH2, MSH6, PMS2, ATM, BRCA1, BRCA2, PALB2, BRIP1, and BARD1.

Embodiment 47. The method of any one of Embodiments 1-46, wherein (f) is based on a homozygous deletion of CDKN2A.

Embodiment 48. The method of any one of Embodiments 1-47, further comprising creating a record associated with the subject designating the subject as (i) one who will likely benefit from the treatment comprising an immune checkpoint inhibitor or (ii) one who will likely not benefit from the treatment comprising an immune checkpoint inhibitor, wherein the designation is based on the composite biomarker score.

Embodiment 49. The method of any one of Embodiments 1-48, further comprising administering the immune checkpoint inhibitor to the identified subject if the subject is determined to be likely to benefit from the treatment comprising the immune checkpoint inhibitor.

Embodiment 50. The method of any one of Embodiments 1-49, wherein the cancer is a lung cancer, a melanoma, a bladder cancer, a gastro-esophageal cancer, or a head and neck cancer.

Embodiment 51. The method of any one of Embodiments 1-50, wherein the immune checkpoint inhibitor is a PD-1 inhibitor.

Embodiment 52. The method of Embodiment 51, wherein the immune checkpoint inhibitor comprises one or more of nivolumab, pembrolizumab, cemiplimab, or dostarlimab.

Embodiment 53. The method of any one of Embodiments 1-50, wherein the immune checkpoint inhibitor is a PD-L1 inhibitor.

Embodiment 54. The method of Embodiment 53, wherein the immune checkpoint inhibitor comprises one or more of atezolizumab, avelumab, or durvalumab.

Embodiment 55. The method of any one of Embodiments 1-50, wherein the immune checkpoint inhibitor is a CTLA-4 inhibitor.

Embodiment 56. The method of Embodiment 55, wherein the immune checkpoint inhibitor comprises ipilimumab.

Embodiment 57. The method of any one of Embodiments 1-56, wherein the tumor sample is a tissue biopsy sample or a liquid biopsy sample.

Embodiment 58. The method of Embodiment 57, wherein the sample is a tissue biopsy and comprises a tumor biopsy or a tumor specimen.

Embodiment 59. The method of Embodiment 57, wherein the sample is a liquid biopsy sample and comprises circulating tumor cells, blood, serum, plasma, cerebrospinal fluid, sputum, stool, urine, or saliva.

Embodiment 60. The method of Embodiment 59, wherein the sample is a liquid biopsy sample and comprises circulating tumor cells (CTCs).

Embodiment 61. The method of Embodiment 59, wherein the sample is a liquid biopsy sample and comprises cell-free DNA (cfDNA), circulating tumor DNA (ctDNA), or both.

Embodiment 62. The method of any one of Embodiments 1-61, wherein the sample comprises cells and/or nucleic acids from the cancer.

Embodiment 63. The method of Embodiment 62, wherein the sample comprises mRNA, DNA, circulating tumor DNA (ctDNA), cell-free DNA, cell-free RNA from the cancer, or any combination thereof.

Embodiment 64. The method of any one of Embodiments 6-63, wherein the LOH status is determined based on sequence read data derived from sequencing nucleic acid molecules extracted from the sample.

Embodiment 65. The method of embodiment 64, wherein the LOH status sequencing comprises use of a massively parallel sequencing (MPS) technique, whole genome sequencing (WGS), whole exome sequencing, targeted sequencing, direct sequencing, next-generation sequencing (NGS), or a Sanger sequencing technique.

Embodiment 66. The method of Embodiment 64 or Embodiment 65, wherein the sequencing comprises: (a) providing a plurality of nucleic acid molecules obtained from the sample, wherein the plurality of nucleic acid molecules comprises a mixture of tumor nucleic acid molecules and non-tumor nucleic acid molecules; (b) optionally, ligating one or more adapters onto one or more nucleic acid molecules from the plurality of nucleic acid molecules; (c) amplifying nucleic acid molecules from the plurality of nucleic acid molecules; (d) optionally, capturing nucleic acid molecules from the amplified nucleic acid molecules, wherein the captured nucleic acid molecules are captured from the amplified nucleic acid molecules by hybridization to one or more bait molecules; and (e) sequencing, by a sequencer, the captured nucleic acid molecules to obtain a plurality of sequence reads corresponding to one or more genomic loci within a subgenomic interval in the sample.

Embodiment 67. The method of embodiment 66, wherein the adapters comprise one or more of amplification primer sequences, flow cell adapter hybridization sequences, unique molecular identifier sequences, substrate adapter sequences, or sample index sequences.

Embodiment 68. The method of Embodiment 66 or Embodiment 67, wherein amplifying nucleic acid molecules comprises performing a polymerase chain reaction (PCR) technique, a non-PCR amplification technique, or an isothermal amplification technique.

Embodiment 69. The method of any one of Embodiments 66-68, wherein the one or more bait molecules comprise one or more nucleic acid molecules, each comprising a region that is complementary to a region of a captured nucleic acid molecule.

Embodiment 70. The method of Embodiment 69, wherein the one or more bait molecules each comprise a capture moiety.

Embodiment 71. The method of Embodiment 70, wherein the capture moiety is biotin.

Embodiment 72. The method of any one of Embodiments 1-71, wherein the subject is a human.

Embodiment 73. A method of treating a subject having a cancer, wherein the subject is identified as one who will benefit from an immune checkpoint inhibitor according to the method of any one of Embodiments 1-72, the method comprising administering the immune checkpoint inhibitor to the subject.

Embodiment 74. The method of any one of Embodiments 1-5, wherein the composite biomarker score is calculated by adding to the score for each positive biomarker and subtracting from the score for each negative biomarker for each of (a)-(g), or (a)-(h), that are met.

Embodiment 75. The method of any one of Embodiments 1-5, wherein each of (a)-(g) or (a)-(h) that are met increments or decreases the score by at least 1.

1. A method of identifying a subject with a cancer who will likely benefit from a treatment comprising an immune checkpoint inhibitor,
the method comprising determining a composite biomarker score;
wherein the composite biomarker score is determined based on at least three of the following (a)-(g):
(a) the tumor mutational burden (TMB) score of a tumor sample associated with the cancer,
(b) the percent of cells in the tumor sample that are positive for PD-L1,
(c) the presence of a cancer-associated mutation in a ARID1A gene in the sample,
(d) the presence of a cancer-associated mutation in a KEAP1/NFE2L2 pathway gene in the sample,
(e) the presence of a cancer-associated mutation in a STK11/LKB1 gene in the sample,
(f) the presence of a cancer-associated mutation in a CDKN2A gene in the sample, and
(g) the presence of a cancer-associated mutation in a DNA damage response (DDR) gene in the sample; and
determining, based on the determined composite biomarker score, whether the subject will likely benefit from the treatment comprising the immune checkpoint inhibitor.

2. The method of claim 1, wherein the method comprises determining a composite biomarker score;
wherein the composite biomarker score is determined based on the following (a)-(g):
(a) the tumor mutational burden (TMB) score of a tumor sample associated with the cancer,
(b) the percent of cells in the tumor sample that are positive for PD-L1,
(c) the presence of a cancer-associated mutation in a ARID1A gene in the sample,
(d) the presence of a cancer-associated mutation in a KEAP1/NFE2L2 pathway gene in the sample,
(e) the presence of a cancer-associated mutation in a STK11/LKB1 gene in the sample,
(f) the presence of a cancer-associated mutation in a CDKN2A gene in the sample, and
(g) the presence of a cancer-associated mutation in a DNA damage response (DDR) gene in the sample; and
determining, based on the determined composite biomarker score, whether the subject will likely benefit from the treatment comprising the immune checkpoint inhibitor.

3. The method of claim 1, wherein the composite biomarker score is based on at least three of (a)-(g) and further based on (h):
(h) one or more human leukocyte antigen (HLA) Class I genes of the cancer are determined to exhibit loss of heterozygosity (LOH).

4. The method of claim 3, wherein the one or more HLA Class I genes are selected from the group consisting of HLA-A, HLA-B, HLA-C, HLA-DP, HLA-DQ, and HLA-DR.

5. The method of claim 1, further comprising characterizing the subject as:
(i) having a low likelihood of benefitting from the treatment comprising the immune checkpoint inhibitor,
(ii) having an indeterminate or medium likelihood of benefitting from the treatment comprising the immune checkpoint inhibitor, or
(iii) having a high likelihood of benefitting from the treatment comprising the immune checkpoint inhibitor;
wherein the subject is characterized as one of (i)-(iii) based on the determined composite biomarker score.

6. The method of claim 1, wherein the composite biomarker score is calculated (i) by adding to the score for each positive biomarker and subtracting from the score for each negative biomarker for each of (a)-(g) that are met or (ii) by adding to the score for each positive biomarker and subtracting from the score for each negative biomarker for each of (a)-(h) that are met, wherein the composite biomarker score is based on at least three of (a)-(g) and further based on (h): (h) one or more human leukocyte antigen (HLA) Class I genes of the cancer are determined to exhibit loss of heterozygosity (LOH).

7. The method of claim 1, wherein each of (a)-(g) that are met increments or decreases the score by at least 1.

8. The method of claim 1, wherein the composite biomarker score starts at 0 and is incremented or decreased for each of (a)-(g) that are met.

9. The method of claim 1, wherein the determination of whether the subject will likely benefit from the treatment comprising the immune checkpoint inhibitor is based on the composite biomarker score being at least a threshold value.

10. The method of claim 1, wherein the composite biomarker score is incremented based on the TMB score of (a) as follows:
(i) if the TMB score is less than 10, the composite biomarker score is not incremented or decreased,
(ii) if the TMB score is at least 10, the composite biomarker score is incremented, and
(iii) if the TMB score is at least 20, the composite biomarker score is further incremented over the increment of (ii).

11. The method of claim 10, wherein the composite biomarker score is incremented by 1 if (ii) is met and incremented by a total of 2 if both (ii) and (iii) are met.

12. The method of claim 1, wherein the composite biomarker score is incremented based on the percent of cells in the sample that are positive for PD-L1 of (b) as follows:
(i) if the percent of cells in the sample that are positive for PD-L1 is less than 1%, the composite biomarker score is not incremented or decreased,
(ii) if the percent of cells in the sample that are positive for PD-L1 is at least 1% and less than 50%, the composite biomarker score is incremented, and
(iii) if the percent of cells in the sample that are positive for PD-L1 is at least 50%, the composite biomarker score is further incremented over the increment of (ii).

13. The method of claim 12, wherein the composite biomarker score is incremented by 1 if (ii) is met and incremented by a total of 2 if both (ii) and (iii) are met.

14. The method of claim 1, wherein if the composite biomarker score is less than the threshold value, the subject is identified as one who will not benefit from treatment with the immune checkpoint inhibitor.

15. The method of claim 1, wherein the DDR genes comprise one or more of ATM, BRCA2, BRIP1, MRE11, POLE, MSH2, PARP1, MLH1, MSH6, PMS2, BRCA1, PALB2, and BARD1; and wherein (g) is based on a cancer-associated mutation reducing gene expression of one or more genes selected from the list consisting of ATM, BRCA2, BRIP1, MRE11, POLE, MSH2, PARP1, MLH1, MSH6, PMS2, BRCA1, PALB2, and BARD1.

16. The method of claim 1, wherein (f) is based on a homozygous deletion of CDKN2A.

17. The method of claim 1, further comprising administering the immune checkpoint inhibitor to the identified subject if the subject is determined to be likely to benefit from the treatment comprising the immune checkpoint inhibitor.

18. The method of claim 1, wherein the cancer is a lung cancer, a melanoma, a bladder cancer, a gastro-esophageal cancer, or a head and neck cancer.

19. The method of claim 1, wherein the immune checkpoint inhibitor is at least one of a PD-1 inhibitor, a PD-L1 inhibitor, or a CTLA-4 inhibitor.

20. The method of claim 1, wherein the tumor sample is a tissue biopsy sample or a liquid biopsy sample.

21. The method of claim 20, wherein the sample is a tissue biopsy and comprises a tumor biopsy or a tumor specimen.

22. The method of claim 20, wherein the sample is a liquid biopsy sample and comprises circulating tumor cells (CTCs).

23. The method of claim 20, wherein the sample is a liquid biopsy sample and comprises cell-free DNA (cfDNA), circulating tumor DNA (ctDNA), or both.

24. The method of claim 1, wherein the subject is a human.

25. A method of treating a subject having a cancer, wherein the subject is identified as one who will benefit from an immune checkpoint inhibitor according to the method of claim 1, the method comprising administering the immune checkpoint inhibitor to the subject.
